(19) United States
(12) Patent Application Publication
(10) Pub. No.: US 2026/0183379 A1
(43) Pub. Date: Jul. 2, 2026

(54) PORCINE SAPOVIRUS ISOLATES AND IMMUNOGENIC COMPOSITIONS THEREFROM

(71) Applicant: Iowa State University Research Foundation, Inc., Ames, IA (US)

(72) Inventors: Jianqiang Zhang, Ames, IA (US); Liying Tian, Ames, IA (US); Ethan Aljets, Ames, IA (US)

(21) Appl. No.: 19/559,682

(22) Filed: Mar. 6, 2026

Related U.S. Application Data
(63) Continuation of application No. PCT/US2024/047729, filed on Sep. 20, 2024.
(60) Provisional application No. 63/539,442, filed on Sep. 20, 2023.
(60) Provisional application No. 63/674,417, filed on Jul. 23, 2024.

(51) Int. Cl. A61K 39/125; A61K 39/00; A61P 31/14; C07K 14/005; C12N 5/071; C12N 7/00; C12Q 1/686; C12Q 1/70

(52) U.S. Cl. CPC A61K 39/125; A61P 31/14; C07K 14/005; C12N 5/0686; C12N 7/00; C12Q 1/686; C12Q 1/701; A61K 2039/5252; A61K 2039/5254; A61K 2039/552; C12N 2770/16022; C12N 2770/16034; C12N 2770/16064

(57) ABSTRACT

The present disclosure is directed to novel porcine sapoviruses (SaV) isolates, including SaV serially propagated in cell culture, all of which are useful in the preparation of immunogenic compositions and vaccines for treating and preventing disease in swine. The disclosure further provides characterization of pathogenicity and cross-protective immunity of contemporary porcine SaV isolates in weaned pigs, demonstrating that prior exposure to homologous strains provides greater protection against viral shedding than heterologous strains.

### CROSS-REFERENCE TO RELATED APPLICATIONS

This application claims priority to provisional applications U.S. Ser. No. 63/539,442, filed Sep. 20, 2023, and U.S. Ser. No. 63/674,417 filed Jul. 23, 2024, which are incorporated herein by reference in their entireties.

### SEQUENCE LISTING XML

The instant application contains a sequence listing, which has been submitted in XML file format by electronic submission and is hereby incorporated by reference in its entirety. The XML file, created on Sep. 19, 2024, is named P14500WO00.xml and is 165,798 bytes in size.

### TECHNICAL FIELD

This disclosure relates generally to the field of vaccines, particularly to novel isolates of porcine sapovirus (SaV) and immunogenic compositions that protect swine from disease caused by SaV.

### BACKGROUND

Sapoviruses (SaVs) are members of the Sapovirus genus in the Caliciviridae family. Sapoviruses possess a single-stranded, positive-sense RNA genome of 7-8 kb in length, which is composed of two overlapping open reading frames (ORFs): ORF1 and ORF2. ORF1 encodes the nonstructural proteins and the capsid protein VP1 while ORF2 encodes the minor structural protein VP2. Sapoviruses are highly genetically diverse and have been classified into 19 genogroups based on the VP1 sequences (GI to GXIX). Sapoviruses mainly cause enteric infections and have been detected in humans and a variety of animal species (e.g., pig, mink, dog, sea lion, bat, chimpanzee, and rat). SaVs in genogroup V have been identified in humans, pigs, and sea lions, suggesting the zoonotic potential of GV SaVs. Thus far, eight genogroups of SaVs (GIII and GV-GXI) have been detected in pigs and GIII SaV appears to be the most frequently detected genogroup in pigs. For porcine sapoviruses, only the Cowden strain has been successfully isolated in cell culture since the first discovery in pigs in the United States in 1980. Unavailability of contemporary porcine SaVs isolates has hampered the characterization of virus growth and replication, virus pathogenicity, and vaccine development.

### SUMMARY

Four contemporary porcine sapovirus (SaV) GIII strains obtained from pig samples collected in the United States in 2022 and 2023 were successfully isolated in cell culture, marking the first such achievement since the Cowden strain. The contemporary porcine SaV isolates of the disclosure provide an important tool for pathogenesis studies, virological and serological assay development, and vaccine development.

The contemporary porcine SaV isolates have been characterized for pathogenicity and cross-protection in weaned pigs under controlled experimental conditions. Studies demonstrate that both IL25663/2022 and IA79982/2022 isolates cause detectable infection with viral shedding lasting approximately 14-21 days post inoculation. The isolates exhibit differences in virulence, with IL25663/2022 inducing higher levels of viral shedding than IA79982/2022. Upon challenge with IL25663/2022, prior exposure to the homologous strain resulted in the greatest reduction in viral shedding, followed by heterologous strain exposure, demonstrating partial cross-protection with more robust immunity observed in homologous challenge. These findings support the existence of strain-specific differences in pathogenicity and immunity, and highlight the utility of the disclosed isolates for vaccine development targeting porcine SaV.

The present disclosure encompasses immunogenic compositions comprising porcine SaV strains. The SaV strains may be used, in one embodiment, as inactivated or live attenuated vaccines. Thus, the disclosure comprises an immunogenic composition, suitable to be used as a vaccine, which comprises a SaV strain of the disclosure, preferably live and attenuated, or an immunogenic fragment thereof, one or more adjuvants, and optionally one or more excipients, in an amount effective to elicit production of neutralizing antibodies in swine.

The immunogenic compositions of the disclosure protect swine from infection by SaV. The present disclosure includes novel nucleotide and amino acid sequences of porcine SaV, including novel genotypes thereof, all of which are useful in the preparation of vaccines for treating and preventing diseases in swine and other animals. Diagnostic and therapeutic polyclonal and monoclonal antibodies are also a feature of the present disclosure, as are infectious clones useful in the propagation of the virus and in the preparation of vaccines. The disclosure also provides the full-length genomic sequences of porcine SaV strains at different passages in cell culture.

The present disclosure provides methods for inducing an immune response against SaV and methods of treating or preventing a disease in an animal caused by infection with SaV, including disease states that are directly caused by SaV, and disease states contributed to or potentiated by porcine SaV. The present disclosure also includes the option to administer a combination vaccine, that is, a bivalent or multivalent combination of antigens, which may include live, modified live, or inactivated antigens against a non-SaV pathogen, with appropriate choice of adjuvant. The present disclosure also provides methods for determining if a population of swine is in need of vaccination against SaV infection.

The present disclosure further provides in vivo characterization of contemporary porcine SaV isolates demonstrating strain-specific differences in pathogenicity and immunity. In some embodiments, prior exposure to a homologous SaV strain provides greater protection against subsequent challenge than prior exposure to a heterologous SaV strain. The disclosure demonstrates that both homologous and heterologous prior exposure to porcine SaV may reduce viral shedding upon challenge, with homologous exposure providing the greatest reduction in viral shedding. These findings support the development of immunogenic compositions and vaccines that may provide cross-protective immunity against multiple porcine SaV strains.

Representative embodiments of the disclosure include an isolated polynucleotide sequence that includes a genomic polynucleotide which encodes SaV proteins which are attenuated and may be used as an immunogenic composition. This can include whole genome sequences selected from:
(a) SEQ ID NO: 1, 2, 3, 4, 21, 22, 23, or 24 or an immunogenic fragment thereof,
(b) the complement of any sequence in (a);
(c) a polynucleotide that hybridizes with a sequence of (a) or (b) under stringent conditions defined as hybridizing to filter bound DNA in 0.5M NaHPO₄, 7% SDS, 1 mM EDTA at 65° C., and washing in 0.1×SSC/0.1% SDS at 68° C.;
(d) a polynucleotide that is at least 70% identical to the polynucleotide of (a) or (b);
(e) a polynucleotide that is at least 80% identical to the polynucleotide of (a) or (b);
(f) a polynucleotide that is at least 90% identical to the polynucleotide of (a) or (b);
(g) a polynucleotide that is at least 95% identical to the polynucleotide of (a) or (b);
(h) a polynucleotide that is at least 98% identical to the polynucleotide of (a) or (b); and
(i) a polynucleotide that is at least 99% identical to the polynucleotide of (a) or (b).

The disclosure further provides RNA and DNA molecules, their complements, fragments and vectors and plasmids for the expression of any such RNA or DNA polynucleotides, and for SaV that is expressed from such nucleotide sequences, wherein said virus is live, or fully or partially attenuated.

Methods of preparing a live attenuated SaV such that the virus fails to cause clinical signs of SaV when administered to a swine but is capable of inducing an immune response that immunizes the swine against pathogenic forms of SaV are also provided.

The disclosure also provides a vaccine that comprises a polynucleotide sequence as aforementioned, and corresponding nucleotide sequences that may function as infectious clones.

The disclosure further provides nucleic acid sequences and resultant protein variants that have amino acid substitutions, and which reduce virulence, cause attenuation and allow the compositions to be used safely as immunogenic compositions and as vaccines. In certain embodiments, the nucleic acid and protein sequences include at least one base or amino acid change such that the sequence is not a naturally occurring sequence.

While multiple embodiments are disclosed, still other embodiments of the present disclosure will become apparent based on the detailed description, which shows and describes illustrative embodiments of the disclosure. Accordingly, the figures and detailed description are to be regarded as illustrative in nature and not restrictive.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the specification and are included to further demonstrate certain embodiments. In some instances, embodiments can be best understood by referring to the accompanying figures in combination with the detailed description presented herein. The description and accompanying figures may highlight a certain specific example, or a certain embodiment. However, one skilled in the art will understand that portions of the example or embodiment may be used in combination with other examples or embodiments.

FIG. 1 shows nucleotide alignment of partial ORF2 (encoding VP2) sequences of SaVs Cowden, IL25663-2/2022 P1 and P5, IA79982-GB/2022 P1 and P3, IL67435-GA/2022 P4, and IA43277-GG/2023 P2 isolates (SEQ ID NOs: 32-36). The stop codon TGA of ORF2 is indicated. The genomic region with insertion or deletion is shown by boxed line.

FIG. 2A-D shows phylogenetic analysis of 19 genogroups of sapoviruses based on the available nearly full-length genome sequences. The contemporary porcine SaV isolates IL25563-2/2022 P1 and P5, IA79982-GB/2022 P1 and P3, IL67435-GA/2022 P4, and IA43277-GG/2023 are shown by grey color bullet points. Porcine SaV Cowden isolate is shown by black color bullet point.

FIG. 3A-D shows phylogenetic analysis of 19 genogroups of sapoviruses based on the VP1 nucleotide sequences. The contemporary porcine SaV isolates IL25563-2/2022 P1 and P5, IA79982-GB/2022 P1 and P3, IL67435-GA/2022 P4, and IA43277-GG/2023 are shown by grey color bullet points. Porcine SaV Cowden isolate is shown by black color bullet point.

FIG. 4A-D shows phylogenetic analysis of 19 genogroups of sapoviruses based on the VP2 nucleotide sequences. The contemporary porcine SaV isolates IL25563-2/2022 P1 and P5, IA79982-GB/2022 P1 and P3, IL67435-GA/2022 P4, and IA43277-GG/2023 are shown by grey color bullet points. Porcine SaV Cowden isolate is shown by black color bullet point.

FIG. 5 shows immunofluorescence staining of porcine SaV-infected LLC-PK1 cells using peptide antisera.

FIG. 6 shows porcine SaV immunohistochemistry (IHC) using peptide antisera.

FIG. 7 shows an indirect fluorescent antibody (IFA) assay for antibody detection using one porcine SaV isolate.

FIG. 8 shows clinical observations including (A) overall average diarrhea scores from −3 to 14 DPI, (B) microchip body temperature of pigs over time, and (C) mean average daily weight gain (ADG) of pigs between different time intervals.

FIG. 9 shows RNA load of porcine rotavirus A and porcine rotavirus C in rectal swab samples as determined by real-time RT-PCR, with (A) showing RVA and (B) showing RVC.

FIG. 10 shows porcine sapovirus RNA load in (A) rectal swab samples and (B) oral fluid samples as determined by real-time RT-PCR, demonstrating viral shedding patterns and cross-protection upon challenge at 35 DPI.

FIG. 11 shows intestinal morphology measurements of pigs necropsied at 4 DPI, including (A) villus length (μm), (B) crypt depth (μm), and (C) villus length/crypt depth ratio.

FIG. 12 shows immunohistochemistry (IHC) scores of pigs necropsied at 4 DPI for (A) porcine sapovirus, (B) porcine rotavirus A, and (C) porcine rotavirus C.

### DETAILED DESCRIPTION

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

It is to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Similarly, the word “or” is intended to include “and” unless the context clearly indicate otherwise. The word “or” means any one member of a particular list and also includes any combination of members of that list. Further, all units, prefixes, and symbols may be denoted in its SI accepted form.

Numeric ranges recited within the specification are inclusive of the numbers defining the range and include each integer within the defined range. Throughout this disclosure, various embodiments of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

The term “adjuvant” refers to a compound that enhances the effectiveness of the vaccine and may be added to the formulation that includes the immunizing agent. Adjuvants provide enhanced immune response even after administration of only a single dose of the vaccine. Adjuvants may include, for example, aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), non-metabolizable oil, mineral and/or plant/vegetable and/or animal oils, polymers, carbomers, surfactants, natural organic compounds, plant extracts, carbohydrates, cholesterol, lipids, water-in-oil emulsion, oil-in-water emulsion, water-in-oil-in-water emulsion, HRA-3 (acrylic acid saccharide cross-linked polymer), HRA-3 with cottonseed oil (CSO), or an acrylic acid polyol cross-linked polymer. The emulsion can be based in particular on light liquid paraffin oil (European Pharmacopeia type); isoprenoid oil such as squalane or squalene; oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. In certain embodiments, the emulsifiers are nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the PLURONIC® brand products, especially L121. See Hunter et al., The Theory and Practical Application of Adjuvants (Ed. Stewart-Tull, D. E. S.) John Wiley and Sons, NY, pp 51-94 (1995) and Todd et al., Vaccine 15:564-570 (1997). In certain embodiments, the adjuvant is at a concentration of about 0.01 to about 50%, at a concentration of about 2% to 30%, at a concentration of about 5% to about 25%, at a concentration of about 7% to about 22%, or at a concentration of about 10% to about 20% by volume of the final product. Examples of suitable adjuvants are described in U.S. Patent Application Publication No. US2004/0213817 A1. “Adjuvanted” refers to a composition that incorporates or is combined with an adjuvant.

“Antibodies” refers to polyclonal and monoclonal antibodies, chimeric, and single chain antibodies, as well as Fab fragments, including the products of a Fab or other immunoglobulin expression library. With respect to antibodies, the term, “immunologically specific” refers to antibodies that bind to one or more epitopes of a protein of interest, but which do not substantially recognize and bind other molecules in a sample containing a mixed population of antigenic biological molecules.

An “attenuated” SaV as used herein refers to a SaV which is capable of infecting and/or replicating in a susceptible host but is non-pathogenic or less-pathogenic to the susceptible host. For example, the attenuated virus may cause no observable/detectable clinical manifestations, or less clinical manifestations, or less severe clinical manifestations, or exhibit a reduction in virus replication efficiency and/or infectivity, as compared with the related field isolated strains. The clinical manifestations of SaV infection can include, without limitation, symptoms of gastroenteritis, i.e., diarrhea.

The term “inactivated” and “inactivated virus” refers to a previously virulent virus that has been irradiated (UV, X-ray, or gamma radiation), heated or chemically treated to inactivate, kill, or otherwise modify the virus to substantially eliminate its virulent properties while retaining its immunogenicity. In certain embodiments, the inactivated viruses disclosed herein are inactivated by treatment with an inactivating agent. Suitable inactivating agents include beta propiolactone, binary or beta-ethyleneimine (BEI), glutaraldehyde, ozone, and Formalin (formaldehyde).

“Diluents” can include water, saline, dextrose, ethanol, glycerol, and the like. Isotonic agents can include sodium chloride, dextrose, mannitol, sorbitol, and lactose, among others. Stabilizers include albumin and alkali salts of ethylendiamintetracetic acid, among others.

An “epitope” is an antigenic determinant that is immunologically active in the sense that once administered to the host, it is able to evoke an immune response of the humoral (B cells) and/or cellular type (T cells). These are particular chemical groups or peptide sequences on a molecule that are antigenic. An antibody specifically binds a particular antigenic epitope on a polypeptide. In the animal, most antigens will present several or even many antigenic determinants simultaneously. Such a polypeptide may also be qualified as an immunogenic polypeptide and the epitope may be identified as described further.

An “immunogenic composition” refers to a composition of matter that comprises at least one antigen, which elicits an immunological response in the host of a cellular and/or antibody-mediated immune response to the composition or vaccine of interest. Usually, an “immunological response” includes but is not limited to one or more of the following effects: the production or activation of antibodies, B cells, helper T cells, suppressor T cells, and/or cytotoxic T cells and/or gamma-delta T cells, directed specifically to an antigen or antigens included in the composition or vaccine of interest. In certain embodiments, the host will display either a therapeutic or protective immunological response such that resistance to new infection will be enhanced and/or the clinical severity of the disease reduced. Such protection will be demonstrated by either a reduction or lack of clinical signs normally displayed by an infected host, a quicker recovery time and/or a lowered duration or bacterial titer in the tissues or body fluids or excretions of the infected host compared to a healthy control. In certain embodiments, the reduction in symptoms is statistically significant when compared to a control.

The term “immunogenic fragment” as used herein refers to a polypeptide or a fragment of a polypeptide, or a nucleotide sequence encoding the same which comprises an allele-specific motif, an epitope or other sequence such that the polypeptide or the fragment will bind an MHC molecule and induce a cytotoxic T lymphocyte (“CTL”) response, and/or a B cell response (for example, antibody production), and/or T-helper lymphocyte response, and/or a delayed type hypersensitivity (DTH) response against the antigen from which the immunogenic polypeptide or the immunogenic fragment is derived. A DTH response is an immune reaction in which T cell-dependent macrophage activation and inflammation cause tissue injury. A DTH reaction to the subcutaneous injection of antigen is often used as an assay for cell-mediated immunity.

An “infectious DNA molecule”, for purposes of the present disclosure, is a DNA molecule that encodes the necessary elements for viral replication, transcription, and translation into a functional virion in a suitable host cell.

The term “isolated” is used to indicate that a cell, peptide, or nucleic acid is separated from its native environment. Isolated peptides and nucleic acids may be substantially pure, i.e. essentially free of other substances with which they may be bound in nature.

For purposes of the present disclosure, the nucleotide sequence of a second polynucleotide molecule (either RNA or DNA) is “homologous” to the nucleotide sequence of a first polynucleotide molecule, or has “identity” to said first polynucleotide molecule, where the nucleotide sequence of the second polynucleotide molecule encodes the same polypeptide as the nucleotide sequence of the first polynucleotide molecule as based on the degeneracy of the genetic code, or when it encodes a polypeptide that is sufficiently similar to the polypeptide encoded by the nucleotide sequence of the first polynucleotide molecule so as to be useful in practicing the present disclosure. Homologous polynucleotide sequence also refers to sense and anti-sense strands, and in all cases to the complement of any such strands. For purposes of the present disclosure, a polynucleotide molecule is useful in practicing the present disclosure, and is therefore homologous or has identity, where it can be used as a diagnostic probe to detect the presence of SaV or viral polynucleotide in a fluid or tissue sample of an infected pig, e.g. by standard hybridization or amplification techniques. Generally, the nucleotide sequence of a second polynucleotide molecule is homologous to the nucleotide sequence of a first polynucleotide molecule if it has at least about 70% nucleotide sequence identity to the nucleotide sequence of the first polynucleotide molecule as based on the BLASTN algorithm (National Center for Biotechnology Information, otherwise known as NCBI, (Bethesda, Md., USA) of the United States National Institute of Health). In a specific example for calculations according to the practice of the present disclosure, reference is made to BLASTP 2.2.6 [Tatusova T A and TL Madden, “BLAST 2 sequences—a new tool for comparing protein and nucleotide sequences.” (1999) FEMS Microbiol Lett. 174:247-250.]. Briefly, two amino acid sequences are aligned to optimize the alignment scores using a gap opening penalty of 10, a gap extension penalty of 0.1, and the “blosum62” scoring matrix of Henikoff and Henikoff (Proc. Nat. Acad. Sci. USA 325 89:10915-10919. 1992). The percent identity is then calculated as: Total number of identical matches×100/divided by the length of the longer sequence+number of gaps introduced into the longer sequence to align the two sequences.

In certain embodiments, a homologous nucleotide sequence has at least 75%, at least 80%, at least 85%, at least 90%, at least 95%, at least 96%, at least 97%, at least 98%, at least 99%, and at least 99.5% nucleotide sequence identity. Since the genetic code is degenerate, a homologous nucleotide sequence can include any number of “silent” base changes, i.e. nucleotide substitutions that nonetheless encode the same amino acid.

A homologous nucleotide sequence can further contain non-silent mutations, i.e. base substitutions, deletions, or additions resulting in amino acid differences in the encoded polypeptide, so long as the sequence remains at least about 70% identical to the polypeptide encoded by the first nucleotide sequence or otherwise is useful for practicing the present disclosure. In this regard, certain conservative amino acid substitutions may be made which are generally recognized not to inactivate overall protein function: such as in regard of positively charged amino acids (and vice versa), lysine, arginine and histidine; in regard of negatively charged amino acids (and vice versa), aspartic acid and glutamic acid; and in regard of certain groups of neutrally charged amino acids (and in all cases, also vice versa), (1) alanine and serine, (2) asparagine, glutamine, and histidine, (3) cysteine and serine, (4) glycine and proline, (5) isoleucine, leucine and valine, (6) methionine, leucine and isoleucine, (7) phenylalanine, methionine, leucine, and tyrosine, (8) serine and threonine, (9) tryptophan and tyrosine, (10) and for example tyrosine, tryptophan and phenylalanine. Amino acids can be classified according to physical properties and contribution to secondary and tertiary protein structure. A conservative substitution is thus recognized in the art as a substitution of one amino acid for another amino acid that has similar properties, and exemplary conservative substitutions may be found in WO 97/09433, page 10, published Mar. 13, 1997 (PCT/GB96/02197, filed Sep. 6, 1996). Alternatively, conservative amino acids can be grouped as described in Lehninger, (Biochemistry, Second Edition; Worth Publishers, Inc. NY:NY (1975), pp. 71-77). Protein sequences can be aligned using both Vector NTI Advance 11.5 and CLUSTAL 2.1 multiple sequence alignment. As used herein the recitation of a particular amino acid or nucleotide sequence shall include all silent mutations with respect to nucleic acid sequence and any and all conservatively modified variants with respect to amino acid sequences.

Homologous nucleotide sequences can be determined by comparison of nucleotide sequences, for example by using BLASTN, above. Alternatively, homologous nucleotide sequences can be determined by hybridization under selected conditions. For example, the nucleotide sequence of a second polynucleotide molecule is homologous to SEQ ID NO:1 (or any other particular polynucleotide sequence) if it hybridizes to the complement of SEQ ID NO:1 under moderately stringent conditions, e.g., hybridization to filter-bound DNA in 0.5 M NaHPO₄, 7% sodium dodecyl sulfate (SDS), 1 mM EDTA at 65° C., and washing in 0.2×SSC/0.1% SDS at 42° C. (see Ausubel et al editors, Protocols in Molecular Biology, Wiley and Sons, 1994, pp. 6.0.3 to 6.4.10), or conditions which will otherwise result in hybridization of sequences that encode a SaV. Modifications in hybridization conditions can be empirically determined or precisely calculated based on the length and percentage of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press: Cold Spring Harbor, N.Y. (1989), pp. 9.47 to 9.51.

In certain embodiments, a second nucleotide sequence is homologous to SEQ ID NO: 1 (or any other sequence disclosed herein) if it hybridizes to the complement of SEQ ID NO: 1 under highly stringent conditions, e.g. hybridization to filter-bound DNA in 0.5 M NaHPO₄, 7% SDS, 1 mM EDTA at 65° C., and washing in 0.1×SSC/0.1% SDS at 68° C., as is known in the art.

It is furthermore to be understood that the isolated polynucleotide molecules and the isolated RNA molecules of the present disclosure include both synthetic molecules and molecules obtained through recombinant techniques, such as by in vitro cloning and transcription.

“Mammals” include any warm-blooded vertebrates of the Mammalia class, including humans. The terms “porcine” and “swine” are used interchangeably herein and refer to any animal that is a member of the family Suidae such as, for example, a pig.

As used herein, “a pharmaceutically acceptable carrier” or “pharmaceutical carrier” includes any and all excipients, solvents, growth media, dispersion media, coatings, adjuvants, stabilizing agents, diluents, preservatives, inactivating agents, antimicrobial, antibacterial and antifungal agents, isotonic agents, adsorption delaying agents, and the like. Such ingredients include those that are safe and appropriate for use in veterinary applications. Pharmaceutically acceptable carriers are typically non-toxic, inert, solid or liquid carriers.

A “susceptible” host as used herein refers to a cell or an animal that can be infected by SaV. When introduced to a susceptible animal, an attenuated SaV may also induce an immunological response against the SaV or its antigen, and thereby render the animal immunity against SaV infection.

The term “vaccine” refers to an antigenic preparation used to produce immunity to a disease, in order to prevent or ameliorate the effects of infection. Vaccines are typically prepared using a combination of an immunologically effective amount of an immunogen together with an adjuvant effective for enhancing the immune response of the vaccinated subject against the immunogen.

Vaccine formulations will contain a “therapeutically effective amount” of the active ingredient, that is, an amount capable of eliciting an induction of an immunoprotective response in a subject to which the composition is administered. In the treatment and prevention of SaV, for example, a “therapeutically effective amount” would be an amount that enhances resistance of the vaccinated subject to new infection and/or reduces the clinical severity of the disease. Such protection will be demonstrated by either a reduction or lack of symptoms normally displayed by a subject infected with SaV, a quicker recovery time and/or a lowered count of virus particles. Vaccines can be administered prior to infection, as a preventative measure against SaV. Alternatively, vaccines can be administered after the subject already has contracted a disease. Vaccines given after exposure to SaV may be able to attenuate the disease, triggering a superior immune response than the natural infection itself.

The present disclosure provides for reduction of the incidence of and/or severity of clinical symptoms associated with SaV infection. In certain embodiments, the severity and/or incidence of clinical symptoms in animals receiving the immunogenic composition of the present disclosure are reduced at least 10% in comparison to animals not receiving such an administration when both groups (animals receiving and animals not receiving the composition) are challenged with or exposed to infection by SaV. In certain embodiments, the incidence or severity is reduced at least 20%, at least 30%, at least 40%, at least 50%, at least 60%, at least 70%, at least 80%, at least 90%, at least 95%, or at least 100%, wherein the animals receiving the composition of the present disclosure exhibit no clinical symptoms, or alternatively exhibit clinical symptoms of reduced severity.

For the purpose of the practice of all aspects of the disclosure, it is well known to those skilled in the art that there is no absolute immunological boundary in immunological assays in regard of animals that are seronegative for exposure to a particular antigen or pathogen, and those that are seropositive (having been exposed to a vaccine or pathogen). Nonetheless, those skilled in the art would recognize that in serum neutralization assays, seropositive animals would generally be detected at least up to a 1:1000 serum dilution, whereas a seronegative animal would be expected not to neutralize at a higher dilution than about 1:20 or 1:10.

### Vaccine and Immunogenic Compositions

The disclosure also relates to an immunogenic composition, suitable to be used as a vaccine, which comprises a SaV strain according to the disclosure. The immunogenic compositions according to the disclosure elicit a specific humoral immune response toward the SaV comprising neutralizing antibodies.

The immunogenic compositions based upon the strains disclosed herein can provide live attenuated viruses which exhibit high immunogenicity while at the same time not producing dangerous pathogenic or lethal effects.

The immunogenic and vaccine compositions of this disclosure are not, however, restricted to any particular type or method of preparation. These include, but are not limited to, infectious DNA vaccines (i.e., using plasmids, vectors or other conventional carriers to directly inject DNA into pigs), live vaccines, modified live vaccines, inactivated vaccines, subunit vaccines, attenuated vaccines, genetically engineered vaccines, etc. These vaccines are prepared by standard methods known in the art.

In certain embodiments, the immunogenic compositions comprise a live attenuated SaV and a pharmaceutically acceptable carrier. The pharmaceutically acceptable carrier can be, e.g., water, a stabilizer, a preservative, culture medium, or a buffer. Immunogenic and vaccine compositions comprising the attenuated SaV of the disclosure can be prepared in the form of a suspension or in a lyophilized form or, alternatively, in a frozen form. If frozen, glycerol or other similar agents may be added to enhance stability when frozen. The advantages of live attenuated viruses, in general, include the presentation of all the relevant immunogenic determinants of an infectious agent in its natural form to the host's immune system, and the need for relatively small amounts of the immunizing agent due to the ability of the agent to multiply in the vaccinated host.

Attenuation of the virus for a live vaccine, so that it is insufficiently pathogenic to substantially harm the vaccinated target animal, may be accomplished by known procedures, including by serial passaging. The following references provide various general methods for attenuation, and are suitable for attenuation or further attenuation of any of the strains of the present disclosure: B. Neuman et al., Journal of Virology, vol. 79, No. 15, pp. 9665-9676, 2005; J. Netland et al., Virology, v 399(1), pp. 120-128, 2010; Y-P Huang et al., “Sequence changes of infectious bronchitis virus isolates in the 3′ 7.3 kb of the genome after attenuating passage in embryonated eggs, Avian Pathology, v. 36 (1), (Abstract), 2007; and S. Hingley et al., Virology, v. 200(1) 1994, pp. 1-10; see U.S. Pat. No. 3,914,408; and Ortego et al., Virology, vol. 308 (1), pp. 13-22, 2003. In some embodiments, the live attenuated SaV is attenuated by passaging in cell culture such that when the attenuated virus is administered to a swine it fails to cause clinical signs of SaV but is capable of inducing an immune response that immunizes the swine against pathogenic forms of SaV. In certain embodiments, the SaV is passaged in LLC-PK1 cells.

Additional genetically engineered vaccines, which are desirable in the present disclosure, are produced by techniques known in the art. Such techniques involve, but are not limited to, further manipulation of recombinant DNA, modification of or substitutions to the amino acid sequences of the recombinant proteins and the like.

Genetically engineered vaccines based on recombinant DNA technology are made, for instance, by identifying alternative portions of the viral gene encoding proteins responsible for inducing a stronger immune or protective response in pigs (e.g., capsid protein VP1, minor structural protein VP2, a nonstructural protein, etc.). Various subtypes or isolates of the viral protein genes can be subjected to the DNA-shuffling method. The resulting heterogeneous chimeric viral proteins can be used broad protecting subunit vaccines. Alternatively, such chimeric viral genes or immuno-dominant fragments can be cloned into standard protein expression vectors, such as the baculovirus vector, and used to infect appropriate host cells (see, for example, O'Reilly et al., “Baculovirus Expression Vectors: A Lab Manual,” Freeman & Co., 1992). The host cells are cultured, thus expressing the desired vaccine proteins, which can be purified to the desired extent and formulated into a suitable vaccine product.

If the clones retain any undesirable natural abilities of causing disease, it is also possible to pinpoint the nucleotide sequences in the viral genome responsible for any residual virulence, and genetically engineer the virus avirulent through, for example, site-directed mutagenesis. Site-directed mutagenesis is able to add, delete or change one or more nucleotides (see, for instance, Zoller et al., DNA 3:479-488, 1984). An oligonucleotide is synthesized containing the desired mutation and annealed to a portion of single stranded viral DNA. The hybrid molecule, which results from that procedure, is employed to transform bacteria. Then double-stranded DNA, which is isolated containing the appropriate mutation, is used to produce full-length DNA by ligation to a restriction fragment of the latter that is subsequently transfected into a suitable cell culture. Ligation of the genome into the suitable vector for transfer may be accomplished through any standard technique known to those of ordinary skill in the art. Transfection of the vector into host cells for the production of viral progeny may be done using any of the conventional methods such as calcium-phosphate or DEAE-dextran mediated transfection, electroporation, protoplast fusion and other well-known techniques (e.g., Sambrook et al., “Molecular Cloning: A Laboratory Manual,” Cold Spring Harbor Laboratory Press, 1989). The cloned virus then exhibits the desired mutation. Alternatively, two oligonucleotides can be synthesized which contain the appropriate mutation. These may be annealed to form double-stranded DNA that can be inserted in the viral DNA to produce full-length DNA.

An immunologically effective amount of the vaccines of the present disclosure is administered to a pig in need of protection against viral infection. The immunologically effective amount or the immunogenic amount that inoculates the pig can be easily determined or readily titrated by routine testing. An effective amount is one in which a sufficient immunological response to the vaccine is attained to protect the pig exposed to the SaV virus. In certain embodiments, the pig is protected to an extent in which one to all of the adverse physiological symptoms or effects of the viral disease are significantly reduced, ameliorated or totally prevented.

The vaccine or immunogenic compositions of the present disclosure can be formulated following accepted convention to include acceptable carriers for animals, such as standard buffers, stabilizers, diluents, preservatives, and/or solubilizers, and can also be formulated to facilitate sustained release. Diluents include water, saline, dextrose, ethanol, glycerol, and the like. Additives for isotonicity include sodium chloride, dextrose, mannitol, sorbitol, and lactose, among others. Stabilizers include albumin, among others. Other suitable vaccine vehicles and additives, including those that are particularly useful in formulating modified live vaccines, are known or will be apparent to those skilled in the art. See, e.g., Remington's Pharmaceutical Science, 18th ed., 1990, Mack Publishing, which is incorporated herein by reference.

The vaccine or immunogenic compositions of the present disclosure may further comprise one or more additional immunomodulatory components such as, e.g., an adjuvant or cytokine, among others. Non-limiting examples of adjuvants that can be used in the vaccine of the present disclosure include the RIBI adjuvant system (Ribi Inc., Hamilton, Mont.), alum, mineral gels such as aluminum hydroxide gel, oil-in-water emulsions, water-in-oil emulsions such as, e.g., Freund's complete and incomplete adjuvants, Block copolymer (CytRx, Atlanta Ga.), QS-21 (Cambridge Biotech Inc., Cambridge Mass.), SAF-M (Chiron, Emeryville Calif.), AMPHIGEN® adjuvant, saponin, Quil A or other saponin fraction, monophosphoryl lipid A, ionic polysaccharides, and Avridine lipid-amine adjuvant. Non-limiting examples of oil-in-water emulsions useful in the vaccine of the disclosure include modified SEAM62 and SEAM 1/2 formulations. Modified SEAM62 is an oil-in-water emulsion containing 5% (v/v) squalene (Sigma), 1% (v/v) SPAN® 85 detergent (ICI Surfactants), 0.7% (v/v) TWEEN® 80 detergent (ICI Surfactants), 2.5% (v/v) ethanol, 200 μg/ml Quil A, 100 μg/ml cholesterol, and 0.5% (v/v) lecithin. Modified SEAM 1/2 is an oil-in-water emulsion comprising 5% (v/v) squalene, 1% (v/v) SPAN® 85 detergent, 0.7% (v/v) Tween 80 detergent, 2.5% (v/v) ethanol, 100 μg/ml Quil A, and 50 μg/ml cholesterol. Other immunomodulatory agents that can be included in the vaccine include, e.g., one or more interleukins, interferons, or other known cytokines. Additional adjuvant systems permit for the combination of both T-helper and B-cell epitopes, resulting in one or more types of covalent T-B epitope linked structures, which may be additionally lipidated, such as those described in WO2006/084319, WO2004/014957, and WO2004/014956.

The vaccine compositions of the disclosure may or may not include adjuvants. In particular, as based on an orally infective virus, the live vaccines of the disclosure may be used adjuvant free, with a sterile carrier. Adjuvants that may be used for oral administration include those based on CT-like immune modulators (rmLT, CT-B, i.e. recombinant-mutant heat labile toxin of E. coli, Cholera toxin-B subunit); or via encapsulation with polymers and alginates, or with mucoadhesives such as chitosan, or via liposomes. In certain embodiments, an adjuvanted or non-adjuvanted vaccine dose at the minimal protective dose through vaccine release may provide between approximately 10 and approximately 10⁶ log₁₀ TCID₅₀ of virus per dose, or higher. “TCID50” refers to “tissue culture infective dose” and is defined as that dilution of a virus required to infect 50% of a given batch of inoculated cell cultures. Various methods may be used to calculate TCID50, including the Spearman-Karber method which is utilized throughout this specification. For a description of the Spearman-Karber method, see B. W. Mahy & H. O. Kangro, Virology Methods Manual, p. 25-46 (1996). Adjuvants, if present, may be provided as emulsions, more commonly if non-oral administration is selected, but should not decrease starting titer by more than 0.7 logs (80% reduction).

In one example, adjuvant components are provided from a combination of lecithin in light mineral oil, and also an aluminum hydroxide component. Details concerning the composition and formulation of Amphigen® (as representative lecithin/mineral oil component) are as follows.

The adjuvant may be provided as a 2 mL dose in a buffered solution further comprising about 5% (v/v) Rehydragel® (aluminum hydroxide gel) and “20% Amphigen” at about 25% final (v/v). Amphigen® is generally described in U.S. Pat. No. 5,084,269 and provides de-oiled lecithin (e.g., soy) dissolved in a light oil, which is then dispersed into an aqueous solution or suspension of the antigen as an oil-in-water emulsion. Amphigen has been improved according to the protocols of U.S. Pat. No. 6,814,971 to provide a so-called “20% Amphigen” component for use in the final adjuvanted vaccine compositions. Thus, a stock mixture of 10% lecithin and 90% carrier oil (DRAKEOL®, Penreco, Karns City, PA) is diluted 1:4 with 0.63% phosphate buffered saline solution, thereby reducing the lecithin and DRAKEOL components to 2% and 18% respectively (i.e. 20% of their original concentrations) Tween 80 and Span 80 surfactants are added to the composition, with representative final amounts being 5.6% (v/v) Tween 80 and 2.4% (v/v) Span 80, wherein the Span is originally provided in the stock DRAKEOL component, and the Tween is originally provided from the buffered saline component, so that mixture of the saline and DRAKEOL components results in the finally desired surfactant concentrations. Mixture of the DRAKEOL/lecithin and saline solutions can be accomplished using an In-Line Slim Emulsifier apparatus, model 405, Charles Ross and Son, Hauppauge, NY, USA.

In certain embodiments, the vaccine composition also includes Rehydragel® LV (about 2% aluminum hydroxide content in the stock material), as additional adjuvant component (available from Reheis, NJ, USA, and ChemTrade Logistics, USA). With further dilution using 0.63% PBS, the final vaccine composition contains the following compositional amounts per 2 mL dose; 5% (v/v) Rehydragel® LV; 25% (v/v) of “20% Amphigen”, i.e. it is further 4-fold diluted); and 0.01% (w/v) of merthiolate.

As is understood in the art, the order of addition of components can be varied to provide the equivalent final vaccine composition. For example, an appropriate dilution of virus in buffer can be prepared. An appropriate amount of Rehydragel® LV (about 2% aluminum hydroxide content) stock solution can then be added, with blending, in order to permit the desired 5% (v/v) concentration of Rehydragel® LV in the actual final product. Once prepared, this intermediate stock material is combined with an appropriate amount of “20% Amphigen” stock (as generally described above, and already containing necessary amounts of Tween 80 and Span 80) to again achieve a final product having 25% (v/v) of “20% Amphigen”. An appropriate amount of 10% merthiolate can finally be added.

The vaccinate compositions permit variation in all of the ingredients, such that the total dose of antigen may be varied by a factor of 100 (up or down) compared to the antigen dose stated above, or by a factor of 10 or less (up or down). Similarly, surfactant concentrations (whether Tween or Span) may be varied by up to a factor of 10, independently of each other, or they may be deleted entirely, with replacement by appropriate concentrations of similar materials, as is well understood in the art.

Rehydragel® concentrations in the final product may be varied, first by the use of equivalent materials available from many other manufacturers (i.e. Alhydrogel®, Brenntag; Denmark), or by use of additional variations in the Rehydragel® line of products such as CG, HPA or HS. Using LV as an example, final useful concentrations thereof including from 0% to 20%, 2-12%, or 4-8%. Similarly, although in certain embodiments the final concentration of Amphigen (expressed as % of “20% Amphigen”) is 25%, this amount may vary from 5-50%, 20-30%, or about 24-26%.

In certain embodiments, the oil used in the adjuvant formulations of the disclosure is a mineral oil. As used herein, the term “mineral oil” refers to a mixture of liquid hydrocarbons obtained from petrolatum via a distillation technique. The term is synonymous with “liquefied paraffin”, “liquid petrolatum” and “white mineral oil.” The term is also intended to include “light mineral oil,” i.e., oil which is similarly obtained by distillation of petrolatum, but which has a slightly lower specific gravity than white mineral oil. See, e.g., Remington's Pharmaceutical Sciences, 18th Edition (Easton, Pa.: Mack Publishing Company, 1990, at pages 788 and 1323). Mineral oil can be obtained from various commercial sources, for example, J. T. Baker (Phillipsburg, Pa.), USB Corporation (Cleveland, Ohio). In certain embodiments, the mineral oil is light mineral oil commercially available under the name DRAKEOL®.

Typically, the oily phase is present in an amount from 50% to 95% by volume, in an amount of greater than 50% to 85%, in an amount from greater than 50% to 60%, or in the amount of greater than 50-52% v/v of the vaccine composition. The oily phase includes oil and emulsifiers (e.g., SPAN® 80, TWEEN® 80 etc), if any such emulsifiers are present.

Non-natural, synthetic emulsifiers suitable for use in the adjuvant formulations also include sorbitan-based non-ionic surfactants, e.g. fatty-acid-substituted sorbitan surfactants (commercially available under the name SPAN® or ARLACEL®), fatty acid esters of polyethoxylated sorbitol (TWEEN®), polyethylene glycol esters of fatty acids from sources such as castor oil (EMULFOR®); polyethoxylated fatty acid (e.g., stearic acid available under the name SIMULSOL® M-53), polyethoxylated isooctylphenol/formaldehyde polymer (TYLOXAPOL®), polyoxyethylene fatty alcohol ethers (BRIJ®); polyoxyethylene nonphenyl ethers (TRITON® N), polyoxyethylene isooctylphenyl ethers (TRITON® X). In certain embodiments, the synthetic surfactants are the surfactants available under the name SPAN® and TWEEN®, such as TWEEN®-80 (Polyoxyethylene (20) sorbitan monooleate) and SPAN©-80 (sorbitan monooleate). Generally speaking, the emulsifier(s) may be present in the vaccine composition in an amount of 0.01% to 40% by volume, 0.1% to 15%, or 2% to 10%.

In certain embodiments, the final vaccine composition contains SP-Oil® and Rehydragel® LV as adjuvants (or other Rehydragel® or Alhydrogel® products), with representative amounts being about 5-20% SP-Oil (v/v) and about 5-15% Rehydragel LV (v/v), or with 5% and 12%, respectively. In this regard it is understood that % Rehydragel refers to percent dilution from the stock commercial product. SP-Oil© is a fluidized oil emulsion with includes a polyoxyethylene-polyoxypropylene block copolymer (Pluronic® L121, BASF Corporation, squalene, polyoxyethylene sorbitan monooleate (Tween®80, ICI Americas) and a buffered salt solution.

It should be noted that the present disclosure may also be practiced using wherein the adjuvant component is only Amphigen®.

In certain embodiments, the final vaccine composition contains TXO as an adjuvant; TXO is generally described in WO 2015/042369. All TXO compositions disclosed therein are useful in the preparation of the vaccine compositions of the disclosure. In TXO, the immunostimulatory oligonucleotide (“T”), preferably an ODN, preferably containing a palindromic sequence, and optionally with a modified backbone, is present in the amount of 0.1 to 5 μg per 50 μl of the vaccine composition (e.g., 0.5-3 μg per 50 μl of the composition, 0.09-0.11 μg per 50 μl of the composition). The polycationic carrier (“X”) is present in the amount of 1-20 μg per 50 μl (e.g., 3-10 μg per 50 μl, about 5 μg per 50 μl). Light mineral oil (“O”) is also a component of the TXO adjuvant.

In certain embodiments, TXO adjuvants are prepared as follows: sorbitan monooleate, MPL-A and cholesterol are dissolved in light mineral oil. The resulting oil solution is sterile filtered; the immunostimulatory oligonucleotide, Dextran DEAE and Polyoxyethylene (20) sorbitan monooleate are dissolved in aqueous phase, thus forming the aqueous solution; and the aqueous solution is added to the oil solution under continuous homogenization thus forming the adjuvant formulation TXO.

All the adjuvant compositions of the disclosure can be used with any of the porcine SaV strains and isolates of the present disclosure.

Additional adjuvants useful in the practice of the disclosure include Prezent-A (see generally United States published patent application US20070298053; and “QCDCRT” or “QCDC”-type adjuvants (see generally United States published patent application US20090324641.

The immunogenic and vaccine compositions of the disclosure can further comprise pharmaceutically acceptable carriers, excipients and/or stabilizers (see e.g. Remington: The Science and practice of Pharmacy, 2005, Lippincott Williams), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as Mercury((o-carboxyphenyl)thio)ethyl sodium salt, octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG), TWEEN® or PLURONICS®.

Vaccines of the present disclosure can optionally be formulated for sustained release of the virus, infectious DNA molecule, plasmid, or viral vector of the present disclosure. Examples of such sustained release formulations include virus, infectious DNA molecule, plasmid, or viral vector in combination with composites of biocompatible polymers, such as, e.g., poly (lactic acid), poly (lactic-co-glycolic acid), methylcellulose, hyaluronic acid, collagen and the like. The structure, selection and use of degradable polymers in drug delivery vehicles have been reviewed in several publications, including A. Domb et al., 1992, Polymers for Advanced Technologies 3: 279-292, which is incorporated herein by reference. Additional guidance in selecting and using polymers in pharmaceutical formulations can be found in texts known in the art, for example M. Chasin and R. Langer (eds), 1990, “Biodegradable Polymers as Drug Delivery Systems” in: Drugs and the Pharmaceutical Sciences, Vol. 45, M. Dekker, NY, which is also incorporated herein by reference. Alternatively, or additionally, the virus, plasmid, or viral vector can be microencapsulated to improve administration and efficacy. Methods for microencapsulating antigens are well-known in the art, and include techniques described, e.g., in U.S. Pat. Nos. 3,137,631; 3,959,457; 4,205,060; 4,606,940; 4,744,933; 5,132,117; and International Patent Publication WO 95/28227, all of which are incorporated herein by reference.

Liposomes can also be used to provide for the sustained release of virus, plasmid, viral protein, or viral vector. Details concerning how to make and use liposomal formulations can be found in, among other places, U.S. Pat. Nos. 4,016,100; 4,452,747; 4,921,706; 4,927,637; 4,944,948; 5,008,050; and 5,009,956, all of which are incorporated herein by reference.

An effective amount of any of the above-described vaccines can be determined by conventional means, starting with a low dose of virus, viral protein plasmid or viral vector, and then increasing the dosage while monitoring the effects. An effective amount may be obtained after a single administration of a vaccine or after multiple administrations of a vaccine. Known factors can be taken into consideration when determining an optimal dose per animal. These include the species, size, age and general condition of the animal, the presence of other drugs in the animal, and the like. In certain embodiments, the actual dosage is chosen after consideration of the results from other animal studies.

One method of detecting whether an adequate immune response has been achieved is to determine seroconversion and antibody titer in the animal after vaccination. The timing of vaccination and the number of boosters, if any, may be determined by a doctor or veterinarian based on analysis of all relevant factors, some of which are described above.

The effective dose amount of virus, protein, infectious nucleotide molecule, plasmid, or viral vector, of the present disclosure can be determined using known techniques, taking into account factors that can be determined by one of ordinary skill in the art such as the weight of the animal to be vaccinated. In certain embodiments, the dose amount of virus of the present disclosure in a vaccine of the present disclosure ranges from about 10¹ to about 10⁹ pfu (plaque forming units), from about 10² to about 10⁸ pfu, or from about 10³ to about 10⁷ pfu. In certain embodiments, the dose amount of a plasmid of the present disclosure in a vaccine of the present disclosure ranges from about 0.1 μg to about 100 mg, from about 1 μg to about 10 mg, or from about 10 μg to about 1 mg. In certain embodiments, the dose amount of an infectious DNA molecule of the present disclosure in a vaccine of the present disclosure ranges from about 0.1 μg to about 100 mg, from about 1 μg to about 10 mg, or from about 10 μg to about 1 mg. In certain embodiments, the dose amount of a viral vector of the present disclosure in a vaccine of the present disclosure ranges from about 10¹ pfu to about 10⁹ pfu, from about 10² pfu to about 10⁸ pfu, or from about 10³ to about 10⁷ pfu. In certain embodiments, a suitable dosage size ranges from about 0.5 ml to about 10 ml, or from about 1 ml to about 5 ml.

Suitable doses for viral protein or peptide vaccines according to the practice of the present disclosure range generally from 1 to 50 micrograms per dose, or higher amounts as may be determined by standard methods, with the amount of adjuvant to be determined by recognized methods in regard of each such substance. In an example of the disclosure relating to vaccination of swine, an optimum age target for the animals is between about 1 and 21 days, which at pre-weening, may also correspond with other scheduled vaccinations such as against Mycoplasma hyopneumoniae. Additionally, a schedule of vaccination for breeding sows would include similar doses, with an annual revaccination schedule.

An example of a clinical indication is for treatment, control and prevention in both breeding sows and gilts pre-farrowing, followed by vaccination of piglets. In a representative example (applicable to both sows and gilts), two 2 mL doses of vaccine will be used, although of course, actual volume of the dose is a function of how the vaccine is formulated, with actual dosing amounts ranging from 0.1 to 5 mL, taking also into account the size of the animals. Single dose vaccination is also appropriate.

In certain embodiments, the first dose may be administered as early as pre-breeding to 5-weeks pre-farrowing, with the second dose administered at about 1-3 weeks pre-farrowing. In certain embodiments, the doses provide an amount of viral material that corresponds to a TCID₅₀ (tissue culture infective dose) of between about 10⁶ and 10⁸, or between about 10⁷ and 5×10⁷, and can be further varied, as is recognized in the art. Booster doses can be given two to four weeks prior to any subsequent farrowings. In certain embodiments, intramuscular vaccination (all doses) is provided, although one or more of the doses could be given subcutaneously. Oral administration is also provided. Vaccination may also be effective in naïve animals, and non-naïve animals as accomplished by planned or natural infections.

In a further example, the sow or gilt is vaccinated intramuscularly or orally at 5-weeks pre-farrowing and then 2-weeks pre-farrowing. Under these conditions, a protective immune response can be demonstrated in SaV-negative vaccinated sows if they develop antibodies (measured, for example, via fluorescent focal neutralization titer from serum samples) with neutralizing activity, and these antibodies are passively transferred to their piglets. The protocols of the disclosure are also applicable to the treatment of already seropositive sows and gilts, and also piglets and boars. Booster vaccinations can also be given, and these may be via a different route of administration. Although in certain embodiments it is preferred to re-vaccinate a mother sow prior to any subsequent farrowings, the vaccine compositions nonetheless can still provide protection to piglets via ongoing passive transfer of antibodies, even if the mother sow was only vaccinated in association with a previous farrowing.

Piglets may be vaccinated as early as day 1 of life. For example, piglets can be vaccinated at day 1, with or without a booster dose at 3 weeks of age, particularly if the parent sow, although vaccinated pre-breeding, was not vaccinated pre-farrowing. Piglet vaccination may also be effective if the parent sow was previously not naïve either due to natural or planned infection. Vaccination of piglets when the mother has neither been previously exposed to the virus, nor vaccinated pre-farrowing may also effective. Boars (typically kept for breeding purposes) may be vaccinated once every 6 months. Variation of the dose amounts is well within the practice of the art. In certain embodiments, the vaccines of the present disclosure are safe for use in pregnant animals (all trimesters) and neonatal swine. In certain embodiments, the vaccines of the disclosure are attenuated to a level of safety (i.e., only transient mild clinical signs or signs normal to neonatal swine) that is acceptable for even the most sensitive animals again including neonatal pigs. It will be appreciated that sows or gilts immunized with the vaccines of the disclosure may passively transfer immunity to piglets, including SaV-specific IgA, which may protect piglets from porcine SaV associated disease (e.g., gastroenteritis). Generally, pigs that are immunized with the vaccines of the disclosure may have a decrease in amount and/or duration or be protected from shedding SaV in their feces, and further, pigs that are immunized with the vaccines of the disclosure may be protected from diarrhea due to SaV, and further, the vaccines of the disclosure may aid in stopping or controlling the SaV transmission cycle.

When provided therapeutically, the vaccine is provided in an effective amount upon the detection of a sign of actual infection. Suitable dose amounts for treatment of an existing infection include between about 10¹ and about 10⁶ log₁₀ TCID₅₀, or higher, of virus per dose (minimum immunizing dose to vaccine release). A composition is said to be “pharmacologically acceptable” if its administration can be tolerated by a recipient. Such a composition is said to be administered in a “therapeutically or prophylactically effective amount” if the amount administered is physiologically significant.

At least one vaccine or immunogenic composition of the present disclosure can be administered by any means that achieve the intended purpose, using a composition as described herein. For example, route of administration of such a composition can be by parenteral, oral, oronasal, intranasal, intratracheal, topical, subcutaneous, intramuscular, transcutaneous, intradermal, intraperitoneal, intraocular, and intravenous administration. In one embodiment of the present disclosure, the composition is administered by intramuscularly. Parenteral administration can be by bolus injection or by gradual perfusion over time. Any suitable device may be used to administer the compositions, including syringes, droppers, needleless injection devices, patches, and the like. The route and device selected for use will depend on the composition of the adjuvant, the antigen, and the subject, and such are well known to the skilled artisan. Administration that is oral, or alternatively, subcutaneous, is preferred. Oral administration may be direct, via water, or via feed (solid or liquid feed). When provided in liquid form, the vaccine may be lyophilized with reconstitution, or provided as a paste, for direct addition to feed (mix in or top dress) or otherwise added to water or liquid feed.

The appropriate dose of the immunogenic composition of the present disclosure depends on several variables such as the formulation, the route of administration, the animal's age, the animal's weight, the time of administration, the excretion rate, and reaction irritability. One of ordinary skill in the art can determine the appropriate dose by administering the antigen to the animal and assaying for an increase or, if applicable, a decrease in the immune response.

The immunogenic compositions may comprise proteins and/or antigens from at least one additional pathogen (“non-SaV”). The additional pathogen may be any pathogen that causes illness and/or an infection in a porcine subject. Exemplary pathogens include, but are not limited to, porcine reproductive and respiratory syndrome virus (PRRSV), Mycoplasma hyopneumoniae, Mycoplasma hyosynoviae, Mycoplasma rhinitis, Clostridium tetani, Clostridium perfringens, porcine parvovirus, Erysipelothrix rhusiopathiae, Leptospira pomona, Leptospira grippotyphosa, Leptospira hardjo, Leptospira canicola, Leptospira icterohaemorrhagiae, Leptospira Bratislava, porcine circovirus (PCV), Lawsonia intracellularis, Escherichia coli, Actinobacillus pleuropneumoniae, Haemophilus parasuis, Salmonella choleraesuis, Salmonella typhimurium, Streptococcus suis, Pasteurella multocida, Bordetella bronchiseptica, Actinobacillus pleuropneumoniae, Serpulina hyodysenteriae, encephalomyocarditis virus, swine influenza virus, transmissible gastroenteritis virus (TGE), swine delta coronavirus, rotavirus diarrhea, foot and mouth disease virus, classical swine fever virus, pseudorabies virus, Japanese encephalitis virus (JEV), encephalomyocarditis virus, or a combination thereof.

Embodiments herein relating to “vaccine compositions” of the disclosure are also applicable to embodiments relating to “immunogenic compositions” of the disclosure, and vice versa.

### Polynucleotides and Polypeptides

Representative embodiments of the disclosure include an isolated polynucleotide sequence that comprises a polynucleotide selected from: (a) SEQ ID NO: 1, 2, 3, 4, 21, 22, 23, or 24; (b) the complement of any sequence in (a); (c) a polynucleotide that hybridizes with a sequence of (a) or (b) under stringent conditions defined as hybridizing to filter bound DNA in 0.5M NaHPO₄, 7% SDS, 1 mM EDTA at 65° C., and washing in 0.1×SSC/0.1% SDS at 68° C.; (d) a polynucleotide that is at least 70% identical to the polynucleotide of (a) or (b); (e) a polynucleotide that is at least 80% identical to the polynucleotide of (a) or (b); (f) a polynucleotide that is at least 90% identical to the polynucleotide of (a) or (b); (g) a polynucleotide that is at least 95% identical to the polynucleotide of (a) or (b); (h) a polynucleotide that is at least 98% identical to the polynucleotide of (a) or (b); and (i) a polynucleotide that is at least 99% identical to the polynucleotide of (a) or (b).

The disclosure also provides a polypeptide encoded by any of the open reading frames of SEQ ID NO: 5, 6, 7, 8, 13, 14, 15, 16, 25, 26, 27, or 28, combinations thereof, or a polypeptide that is at least 70%, at least 80%, at least 90%, at least 95%, at least 98%, or at least 99% identical thereto, domains thereof, or to a fragment thereof, including the option that additional otherwise identical amino acids are replaced by conservative substitutions.

The disclosure also provides a polypeptide encoded by any of the open reading frames of the SaV strains of the disclosure as set forth in SEQ ID NO: 9, 10, 11, 12, 17, 18, 19, 20, 29, 30, or 31, or a polypeptide that is at least 70%, at least 80%, at least 90%, at least 95%, at least 98%, or at least 99% identical thereto, or to a fragment thereof, including the option that additional otherwise identical amino acids are replaced by conservative substitutions.

The polynucleotide and amino acid sequence information provided by the present disclosure also makes possible the systematic analysis of the structure and function of the viral genes and their encoded gene products. Knowledge of a polynucleotide encoding a viral gene product of the disclosure also makes available anti-sense polynucleotides which recognize and hybridize to polynucleotides encoding a polypeptide of the disclosure, or a fragment thereof. Full length and fragment anti-sense polynucleotides are useful in this respect. The worker of ordinary skill will appreciate that fragment anti-sense molecules of the disclosure include (i) those which specifically recognize and hybridize to a specific RNA (as determined by sequence comparison of DNA encoding a viral polypeptide of the disclosure as well as (ii) those which recognize and hybridize to RNA encoding variants of the encoded proteins. Antisense polynucleotides that hybridize to RNA/DNA encoding other SaV peptides are also identifiable through sequence comparison to identify characteristic, or signature sequences for the family of molecules, further of use in the study of antigenic domains in SaV polypeptides, and may also be used to distinguish between infection of a host animal with remotely related non-SaV members of the Caliciviridae family.

### Antibodies

Also contemplated by the present disclosure are anti-SaV antibodies (e.g., monoclonal and polyclonal antibodies, single chain antibodies, chimeric antibodies, humanized, human, porcine, and CDR-grafted antibodies, including compounds which include CDR sequences which specifically recognize a SaV polypeptide of the disclosure. The term “specific for” indicates that the variable regions of the antibodies of the disclosure recognize and bind a SaV polypeptide exclusively (i.e., are able to distinguish a single SaV polypeptide from related polypeptides despite sequence identity, homology, or similarity found in the family of polypeptides), and which are permitted (optionally) to interact with other proteins (for example, S. aureus protein A or other antibodies in ELISA techniques) through interactions with sequences outside the variable region of the antibodies, and in particular, in the constant region of the Ab molecule. Screening assays to determine binding specificity of an antibody of the disclosure are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6. Antibodies that recognize and bind fragments of the SaV polypeptides of the disclosure are also contemplated, provided that the antibodies are first and foremost specific for, as defined above, a SaV polypeptide of the disclosure from which the fragment was derived.

For the purposes of clarity, “antibody” refers to an immunoglobulin molecule that can bind to a specific antigen as the result of an immune response to that antigen. Immunoglobulins are serum proteins composed of “light” and “heavy” polypeptide chains having “constant” and “variable” regions and are divided into classes (e.g., IgA, IgD, IgE, IgG, and IgM) based on the composition of the constant regions. Antibodies can exist in a variety of forms including, for example, as, Fv, Fab′, F(ab′)₂, as well as in single chains, and include synthetic polypeptides that contain all or part of one or more antibody single chain polypeptide sequences.

### Diagnostic Kits

The present disclosure also provides diagnostic kits. The kit can be valuable for differentiating between porcine animals naturally infected with a field strain of a SaV and porcine animals administered with any of the SaV vaccine or immunogenic compositions described herein. The kits can also be of value because animals potentially infected with field strains of SaV virus can be detected prior to the existence of clinical symptoms and removed from the herd, or kept in isolation away from naive or vaccinated animals. The kits include reagents for analyzing a sample from a porcine animal for the presence of antibodies to a particular component of a specified SaV virus. Diagnostic kits of the present disclosure can include as a component a peptide or peptides from the SaV strains of the disclosure which is present in a field strain but not in the vaccine or immunogenic composition of interest, or vice versa, and selection of such suitable peptide domains is made possible by the extensive amino acid sequencing. As is known in the art, kits of the present disclosure can alternatively include as a component a peptide which is provided via a fusion protein. The term “fusion peptide” or “fusion protein” for purposes of the present disclosure means a single polypeptide chain consisting of at least a portion of a SaV protein and a heterologous peptide or protein.

### EMBODIMENTS

The following numbered embodiments also form part of the present disclosure:
1. An immunogenic composition comprising an inactivated or live attenuated porcine sapovirus (SaV), wherein the SaV comprises a polynucleotide having at least 90%, 95%, 96%, 97%, 98%, 99%, or 99.5% sequence identity to SEQ ID NO: 1, 2, 3, 4, 21, 22, 23, or 24; and a pharmaceutically acceptable carrier.
2. The immunogenic composition of embodiment 1, wherein the SaV is inactivated.
3. The immunogenic composition of embodiment 1 or embodiment 2, wherein the SaV is a live attenuated virus.
4. The immunogenic composition of any one of embodiments 1-3, wherein the SaV is attenuated by passaging in cell culture such that when the attenuated virus is administered to a swine it fails to cause clinical signs of SaV but is capable of inducing an immune response that immunizes the swine against pathogenic forms of SaV.
5. The immunogenic composition of any one of embodiments 1-4, wherein the pharmaceutically acceptable carrier is a diluent, adjuvant, antimicrobial agent, preservative, inactivating agent, or a combination thereof.
6. The immunogenic composition of any one of embodiments 1-5, wherein the SaV is not wild type
7. The immunogenic composition of any one of embodiments 1-6, further comprising one or more non-SaV inactivated or attenuated pathogens or antigenic material thereof.
8. A method for inducing an immune response against porcine sapovirus (SaV) in a swine comprising: administering to the swine an immunogenic composition comprising an inactivated or live attenuated porcine SaV, wherein the SaV comprises a polynucleotide having at least 90%, 95%, 96%, 97%, 98%, 99%, or 99.5% sequence identity to SEQ ID NO: 1, 2, 3, 4, 21, 22, 23, or 24.
9. The method of embodiment 8, wherein the SaV is inactivated.
10. The method of embodiment 8 or embodiment 9, wherein the SaV is a live attenuated virus.
11. The method of any one of embodiments 8-10, wherein the SaV is attenuated by passaging in cell culture such that when the attenuated SaV is administered to a swine it fails to cause clinical signs of SaV but is capable of inducing an immune response that immunizes the swine against pathogenic forms of SaV.
12. The method of any one of embodiments 8-11, wherein the immunogenic composition further comprises a pharmaceutically acceptable carrier.
13. The method of any one of embodiments 8-12, wherein the pharmaceutically acceptable carrier comprises a diluent, adjuvant, antimicrobial agent, preservative, inactivating agent, or a combination thereof.
14. The method of any one of embodiments 8-13, wherein the immunogenic composition further comprises one or more non-SaV inactivated or attenuated pathogens or antigenic material thereof.
15. The method of any one of embodiments 8-14, wherein the swine is a sow, gilt, boar, hog, or piglet.
16. An isolated porcine sapovirus (SaV) comprising a polynucleotide having at least 90%, 95%, 96%, 97%, 98%, 99%, or 99.5% sequence identity to SEQ ID NO: 1, 2, 3, 4, 21, 22, 23, or 24, and wherein the SaV is not wild type.
17. A DNA polynucleotide encoding a porcine sapovirus (SaV), or an immunogenic fragment thereof, wherein the SaV comprises a polynucleotide having at least 90%, 95%, 96%, 97%, 98%, 99%, or 99.5% sequence identity to SEQ ID NO: 1, 2, 3, 4, 21, 22, 23, or 24.
18. A vector comprising the DNA polynucleotide of embodiment 17.
19. A host cell comprising the DNA polynucleotide of embodiment 17 or the vector of embodiment 18.
20. A culture of cells comprising a porcine sapovirus (SaV), wherein the SaV comprises a polynucleotide having at least 90%, 95%, 96%, 97%, 98%, 99%, or 99.5% sequence identity to SEQ ID NO: 1, 2, 3, 4, 21, 22, 23, or 24.
21. The culture of cells of embodiment 20, wherein the cells are LLC-PK1 cells.
22. The culture of cells of embodiment 20 or embodiment 21, wherein the SaV is Sapovirus_Pig/USA/IL25663-2/2022, Sapovirus_Pig/IA79982-GB/2022, Sapovirus_Pig/USA/IL67435/2022, or Sapovirus_Pig/USA/IA43277-GG/2023, wherein a representative culture has been deposited under ATCC Accession No. ______.
23. A method of preparing a live attenuated porcine sapovirus (SaV) comprising: passaging a SaV in cell culture, wherein the SaV comprises a polynucleotide having at least 90%, 95%, 96%, 97%, 98%, 99%, or 99.5% sequence identity to SEQ ID NO: 1, 2, 3, 4, 21, 22, 23, or 24.
24. The method of embodiment 23, wherein the virus is passaged in LLC-PK1 cells.
25. The method of embodiment 23 or embodiment 24, wherein the SaV is Sapovirus_Pig/USA/IL25663-2/2022, Sapovirus_Pig/IA79982-GB/2022, Sapovirus_Pig/USA/IL67435/2022, or Sapovirus_Pig/USA/IA43277-GG/2023, wherein a representative culture has been deposited under ATCC Accession No. ______.
26. A vaccine composition comprising an inactivated or live attenuated porcine sapovirus (SaV), wherein the SaV comprises a polynucleotide having at least 90%, 95%, 96%, 97%, 98%, 99%, or 99.5% sequence identity to SEQ ID NO: 1, 2, 3, 4, 21, 22, 23, or 24; and a pharmaceutically acceptable carrier.
27. The vaccine composition of embodiment 26, wherein the SaV is inactivated.
28. The vaccine composition of embodiment 26 or embodiment 27, wherein the SaV is a live attenuated virus.
29. The vaccine composition of any one of embodiments 26-28, wherein the SaV is attenuated by passaging in cell culture such that when the attenuated virus is administered to a swine it fails to cause clinical signs of SaV but is capable of inducing an immune response that immunizes the swine against pathogenic forms of SaV.
30. The vaccine composition of any one of embodiments 26-29, wherein the pharmaceutically acceptable carrier comprises a diluent, adjuvant, antimicrobial agent, preservative, inactivating agent, or a combination thereof.
31. The vaccine composition of any one of embodiments 26-30, wherein the composition comprises an adjuvant.
32. The vaccine composition of any one of embodiments 26-31, further comprising one or more non-SaV inactivated or attenuated pathogens or antigenic material thereof.
33. The vaccine composition of any one of embodiments 24-32, wherein the vaccine is effective in a single dose program or in a two-dose program.
34. A method of treating or preventing disease caused by porcine sapovirus (SaV) comprising: administering to a swine in need thereof a vaccine composition comprising an inactivated or live attenuated porcine SaV, wherein the SaV comprises a polynucleotide having at least 90%, 95%, 96%, 97%, 98%, 99%, or 99.5% sequence identity to SEQ ID NO: 1, 2, 3, 4, 21, 22, 23, or 24.
35. The method of embodiment 34, wherein the SaV is inactivated.
36. The method of embodiment 34 or embodiment 35, wherein the SaV is a live attenuated virus.
37. The method of any one of embodiments 34-36, wherein the SaV is attenuated by passaging in cell culture such that when the attenuated SaV is administered to a swine it fails to cause clinical signs of SaV but is capable of inducing an immune response that immunizes the swine against pathogenic forms of SaV.
38. The method of any one of embodiments 34-37, wherein the vaccine composition further comprises a pharmaceutically acceptable carrier.
39. The method of any one of embodiments 34-38, wherein the pharmaceutically acceptable carrier is a diluent, adjuvant, antimicrobial agent, preservative, inactivating agent, or a combination thereof.
40. The method of any one of embodiments 34-39, wherein the composition comprises an adjuvant.
41. The method of any one of embodiments 34-40, wherein the vaccine composition further comprises one or more non-SaV inactivated or attenuated pathogens or antigenic material thereof.
42. The method of any one of embodiments 34-41, wherein the swine is a sow, gilt, boar, hog, or piglet.
43. A method for determining if a population of swine is in need of vaccination against porcine sapovirus (SaV) infection: collecting a biological sample from one or more swine of the population; and detecting the presence of the SaV in the biological sample, wherein the SaV comprises a polynucleotide having at least 90%, 95%, 96%, 97%, 98%, 99%, or 99.5% sequence identity to SEQ ID NO: 1, 2, 3, 4, 21, 22, 23, or 24.
44. The method of embodiment 43, wherein the detecting is by polymerase chain reaction, immunohistochemistry, immunofluorescence, or an indirect fluorescent antibody assay.
45. The method of embodiment 43 or embodiment 44, further comprising: administering an effective amount of the vaccine composition of any one of embodiments 26-33 to the population if the SaV is detected.
46. A method for inducing a cross-protective immune response against porcine sapovirus (SaV) in a swine comprising: administering to the swine an immunogenic composition comprising an inactivated or live attenuated porcine SaV, wherein the SaV comprises a polynucleotide having at least 90%, 95%, 96%, 97%, 98%, 99%, or 99.5% sequence identity to SEQ ID NO: 1, 2, 3, 4, 21, 22, 23, or 24; wherein the immunogenic composition induces an immune response that provides protection against challenge with a heterologous SaV strain.
47. The method of embodiment 46, wherein prior exposure to a homologous SaV strain provides greater reduction in viral shedding upon challenge than prior exposure to a heterologous SaV strain.
48. A method of reducing viral shedding of porcine sapovirus (SaV) in a swine comprising: administering to the swine an immunogenic composition comprising an inactivated or live attenuated porcine SaV, wherein the SaV comprises a polynucleotide having at least 90%, 95%, 96%, 97%, 98%, 99%, or 99.5% sequence identity to SEQ ID NO: 1, 2, 3, 4, 21, 22, 23, or 24; wherein the immunogenic composition reduces viral shedding upon subsequent exposure to SaV.
49. The method of embodiment 48, wherein viral shedding is reduced in rectal swabs, oral fluids, or both.
50. The method of embodiment 48 or embodiment 49, wherein the duration of viral shedding is reduced to less than 21 days post-exposure.
51. The immunogenic composition of claim 50, wherein the IL25663/2022 strain induces higher levels of viral shedding than the IA79982/2022 strain.
52. A method of characterizing pathogenicity of a porcine sapovirus (SaV) strain comprising:
   inoculating weaned pigs with the SaV strain;
   measuring viral shedding in rectal swabs and oral fluid samples over time;
   performing immunohistochemistry on intestinal tissues; and determining villus length, crypt depth, and villus/crypt ratio in intestinal tissues.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, it will be obvious that certain changes and modifications may be practiced within the scope of the appended embodiments.

The following examples are offered by way of illustration and not by way of limitation.

### EXAMPLES

### Example 1: Isolation and Characterization of Porcine Sapovirus Genogroup III

Porcine SaV virus isolation was attempted from over 30 porcine SaV GIII PCR-positive samples in various cell lines. Eventually, the isolation protocol was optimized and porcine SaV GIII was successfully isolated in LLC-PK1 (ATCC CCL-101) cells. Briefly, LLC-PK1 cells were cultured in MEM supplemented with 10% fetal bovine serum, 1% L-glutamine, 1% non-essential amino acids (NEAA), and 1× antibiotics (0.05 mg/ml gentamicin, 100 Unit/mL penicillin, 100 μg/mL streptomycin, and 0.25 μg/mL amphotericin). When LLC-PK1 cells grown in T25 flasks reached about 70% confluence, culture medium was removed and cells were washed three times with the washing medium (MEM with 1% L-glutamine and 1% antibiotics). Subsequently, clinical samples filtered through 0.22 micron filters were added (0.5 ml per flask) and the flasks were incubated at 37° C. for 1 hour with rocking cells every 15 min. After that, the inoculation medium (washing medium with 100 μM of taurochenodeoxycholic acid [TCDCA] and 100 μM of sodium glycochenodeoxycholate [GCDCA]) was added to the inoculated cells (5 ml per flask). The cells were incubated at 37° C. Cells were checked daily for development of cytopathic effects (CPE). After incubation for 2-4 days, flasks were free-thawed once and the supernatants were collected. The supernatants were used for next passages in LLC-PK1 cells. After serial passages, the cell culture supernatants were tested by SaV real-time RT-PCR to confirm the outcome of virus isolation. Eight porcine SaV isolates were successfully obtained and four isolates (Pig/USA/IL25663-2/2022, Pig/USA/IA79982-GB/2022, Pig/USA/IL67435-GA/2022, and Pig/USA/IA43277-GG/2023) were further characterized.

Four sapovirus isolates were serially propagated in cell culture for different passages (IL25663-2/2022: P0 to P5; IA79982-GB/2022: P0 to P3; IL67435/2022: P0 to P4; and IA43277-GG/2023: P0 to P2). Cytopathic effects characterized by cell rounding and detachment were observed. The PCR Ct values of the isolate IL25663-2/2022 at passage 2 (P2), P3, P4 and P5 were 13.5, 13.6, 12.4 and 12.0, respectively. The PCR Ct values of the isolate IA79982-GB/2022 at P0, P1, P2 and P3 were 14.1, 13.6, 12.7, and 11.8, respectively. The PCR Ct values of the isolate IL67435-GA/2022 at P0, P1, P2, P3 and P4 were 14.7, 18.1, 14.6, 9.8 and 9.1, respectively. The PCR Ct values of the isolate IA43277-GG/2023 at P0, P1 and P2 were 12.7, 11.5 and 11.4, respectively.

The entire genome sequences were determined for the four virus isolates at different passages (P1 and P5 for the isolate IL25663-2/2022; P1 and P3 for the isolate IA79982-GB/2022; P4 for the isolate IL67435-GA/2022; and P2 for the isolate IA43277-GG/2023) using the next generation sequencing (NGS) technology. The sequences of these four SaV GIII isolates were compared to the prototype porcine SaV GIII Cowden isolate. The full-length genomes of Cowden, IL25663-2/2022 P1, IL25663-2/2022 P5, IA79982-GB/2022 P1, IA79982-GB/2022 P3, IL67435-GA/2022 P4, and IA43277-GG/2023 P2 isolates were 7319, 7340, 7340, 7349, 7349, 7340, and 7340 nucleotides (nt) in length, respectively. The nucleotide identities between these sapovirus isolates at the whole genome level, VP1 genomic region, and VP2 genomic regions are summarized in Tables 1-3. Table 1 shows the nucleotide identity of four contemporary porcine sapovirus GIII isolates and the prototype GIII Cowden isolate at the whole genome level. Table 2 shows the nucleotide identity of four contemporary porcine sapovirus GIII isolates and the prototype GIII Cowden isolate at the VP1 nucleotide level. Table 3 shows the nucleotide identity of four contemporary porcine sapovirus GIII isolates and the prototype GIII Cowden isolate at the VP2 nucleotide level.

TABLE 1

| | AF182760_{—} | SaV_pig/ | SaV_pig/ | SaV_pig/ | SaV_pig/ | SaV_pig/ | SaV_pig/ |
|---|---|---|---|---|---|---|---|
| | SaV_pig/ | IL25663- | IL25663- | IA79982- | IA79982- | IL67435- | IA43277- |
| | Cowden | 2/2022_P1 | 2/2022_P5 | GB/2022_P1 | GB/2022_P3 | GA/2022_P4 | GG/2023_P2 |
| AF182760_SaV_{—} | 100.00% | 83.56% | 83.56% | 83.49% | 83.49% | 83.76% | 83.69% |
| pig/Cowden | | | | | | | |
| SaV_pig/IL25663- | | 100.00% | 100.00% | 89.63% | 89.63% | 93.14% | 92.63% |
| 2/2022_P1 | | | | | | | |
| SaV_pig/IL25663- | | | 100.00% | 89.63% | 89.63% | 93.14% | 92.63% |
| 2/2022_P5 | | | | | | | |
| SaV_pig/IA79982- | | | | 100.00% | 100.00% | 91.25% | 90.72% |
| GB/2022_P1 | | | | | | | |
| SaV_pig/IA79982- | | | | | 100.00% | 91.25% | 90.72% |
| GB/2022_P3 | | | | | | | |
| SaV_pig/IL67435- | | | | | | 100.00% | 95.48% |
| GA/2022_P4 | | | | | | | |
| SaV_pig/IA43277- | | | | | | | 100.00% |
| GG/2023_P2 | | | | | | | |

TABLE 2

| | AF182760_{—} | SaV_pig/ | SaV_pig/ | SaV_pig/ | SaV_pig/ | SaV_pig/ | SaV_pig/ |
|---|---|---|---|---|---|---|---|
| | SaV_pig/ | IL25663- | IL25663- | IA79982- | IA79982- | IL67435- | IA43277- |
| | Cowden | 2/2022_P1 | 2/2022_P5 | GB/2022_P1 | GB/2022_P3 | GA/2022_P4 | GG/2023_P2 |
| AF182760_SaV_{—} | 100.00% | 85.06% | 85.06% | 84.08% | 84.08% | 84.63% | 84.32% |
| pig/Cowden | | | | | | | |
| SaV_pig/IL25663- | | 100.00% | 100.00% | 81.26% | 81.26% | 93.45% | 92.41% |
| 2/2022_P1 | | | | | | | |
| SaV_pig/IL25663- | | | 100.00% | 81.26% | 81.26% | 93.45% | 92.41% |
| 2/2022_P5 | | | | | | | |
| SaV_pig/IA79982- | | | | 100.00% | 100.00% | 81.69% | 81.81% |
| GB/2022_P1 | | | | | | | |
| SaV_pig/IA79982- | | | | | 100.00% | 81.69% | 81.81% |
| GB/2022_P3 | | | | | | | |
| SaV_pig/IL67435- | | | | | | 100.00% | 95.53% |
| GA/2022_P4 | | | | | | | |
| SaV_pig/IA43277- | | | | | | | 100.00% |
| GG/2023_P2 | | | | | | | |

TABLE 3

| | AF182760_{—} | SaV_pig/ | SaV_pig/ | SaV_pig/ | SaV_pig/ | SaV_pig/ | SaV_pig/ |
|---|---|---|---|---|---|---|---|
| | SaV_pig/ | IL25663- | IL25663- | IA79982- | IA79982- | IL67435- | IA43277- |
| | Cowden | 2/2022_P1 | 2/2022_P5 | GB/2022_P1 | GB/2022_P3 | GA/2022_P4 | GG/2023_P2 |
| AF182760_SaV_{—} | 100.00% | 86.46% | 86.46% | 86.06% | 86.06% | 86.06% | 86.67% |
| pig/Cowden | | | | | | | |
| SaV_pig/IL25663- | | 100.00% | 100.00% | 85.86% | 85.86% | 91.92% | 92.12% |
| 2/2022_P1 | | | | | | | |
| SaV_pig/IL25663- | | | 100.00% | 85.86% | 85.86% | 91.92% | 92.12% |
| 2/2022_P5 | | | | | | | |
| SaV_pig/IA79982- | | | | 100.00% | 100.00% | 85.66% | 86.26% |
| GB/2022_P1 | | | | | | | |
| SaV_pig/IA79982- | | | | | 100.00% | 85.66% | 86.26% |
| GB/2022_P3 | | | | | | | |
| SaV_pig/IL67435- | | | | | | 100.00% | 95.15% |
| GA/2022_P4 | | | | | | | |
| SaV_pig/IA43277- | | | | | | | 100.00% |
| GG/2023_P2 | | | | | | | |

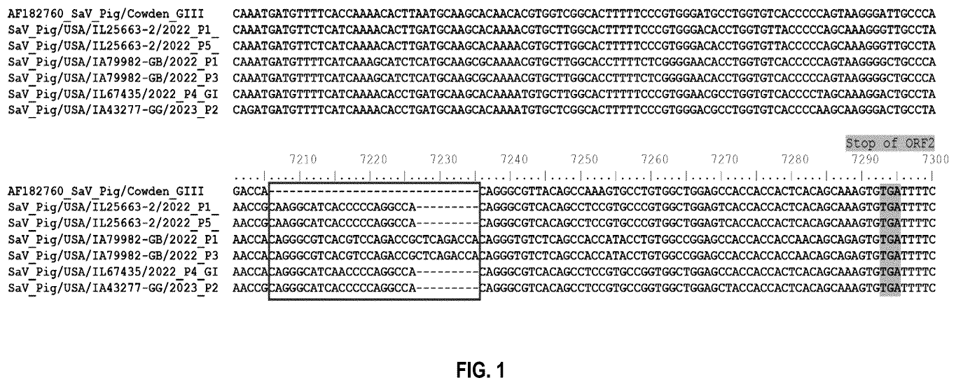

The IL25663-2/2022 P1 and P5 had 10000 nt identity to each other, 89.630% nt identity to IA79982-GB/2022 P1 and P3, 93.14% nt identity to IL67435-GA/2022 P4, 92.63% nt identity to IA43277-GG/2023 P2, and 83.56% nt identity to the Cowden isolates at the whole genome level. Similarly, the IA79982-GB/2022 P1 and P3 had 100% nt identity to each other, 91.25% nt identity to IL67435-GA/2022 P4, 90.72% nt identity to IA43277-GG/2023, and 83.49% nt identity to the Cowden isolate at the whole gnome level. The IL67435-GA/2022 P4 isolate had 95.4800 nt identity to the IA43277-GG isolate at the whole genome level. Interestingly, within ORF2 genomic region, SaVs IL25663-2/2022 P1 and P5, IL67435-GA/2022 P4, and IA43277-GG2023 had a continuous 21-nt insertion and IA79982-GB/2022 P1 and P3 had a continuous 30-nt insertion when compared to the Cowden isolate (FIG. 1). The IA79982-GB/2022 isolate had a continuous 9-nt insertion compared to the IL25563-2/2022, IL67435-GA/2022, and IA43277-GG/2023 isolates (FIG. 1).

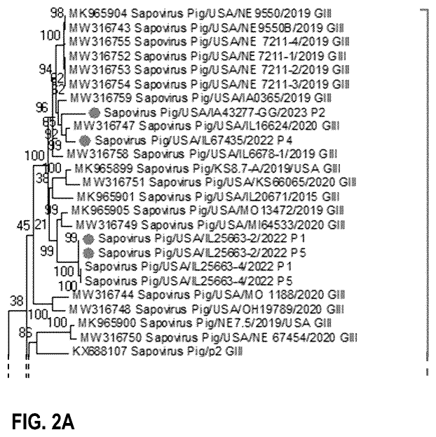

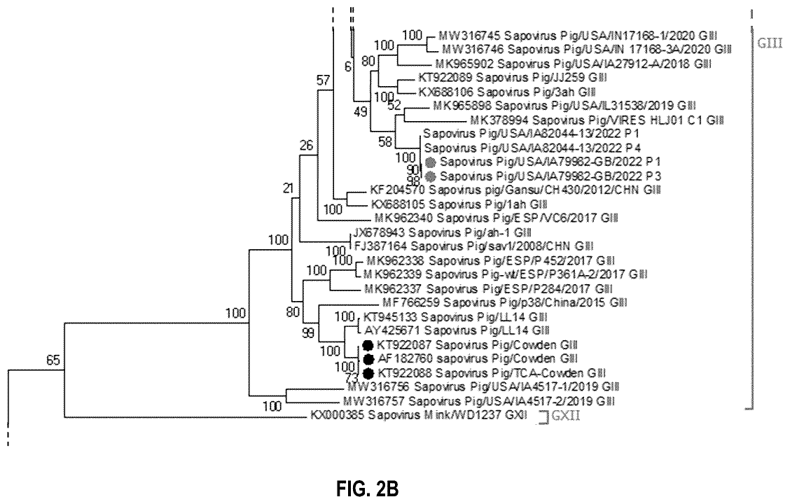

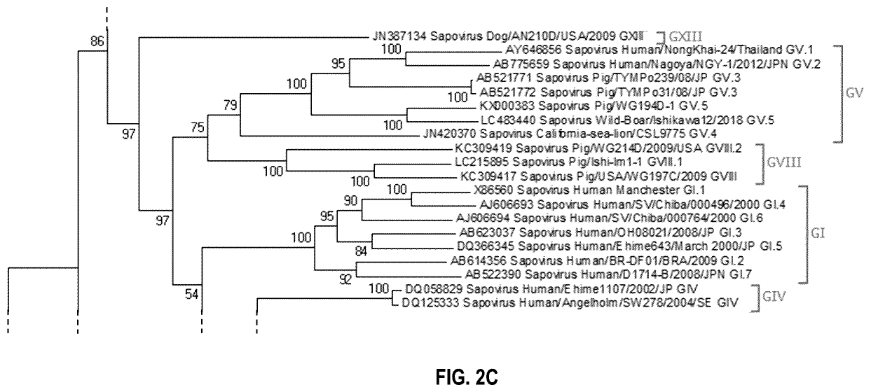

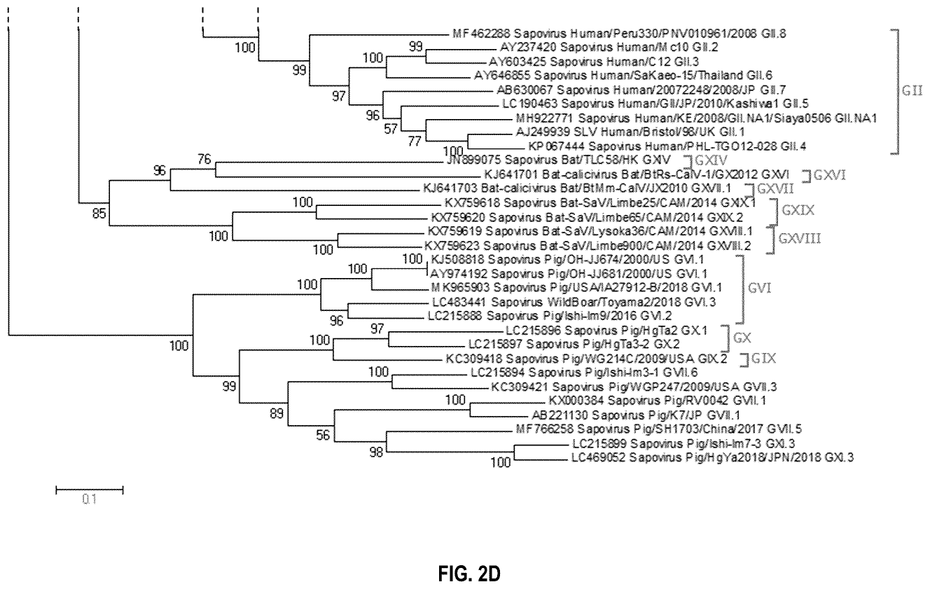

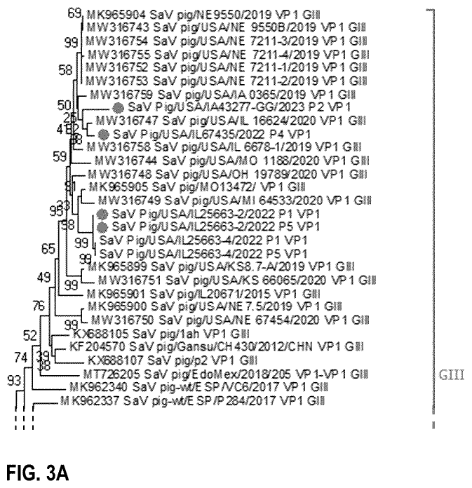

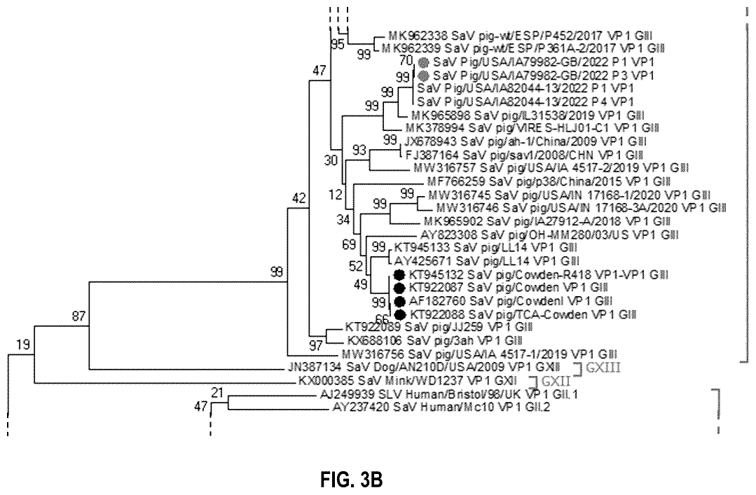

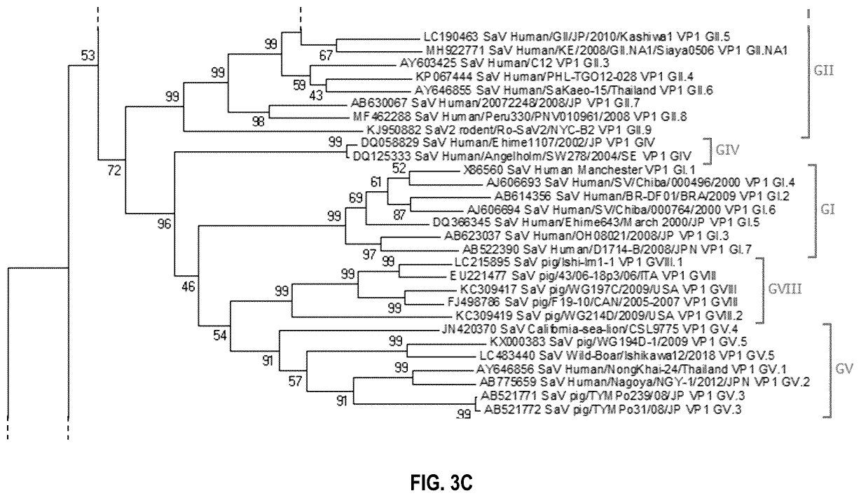

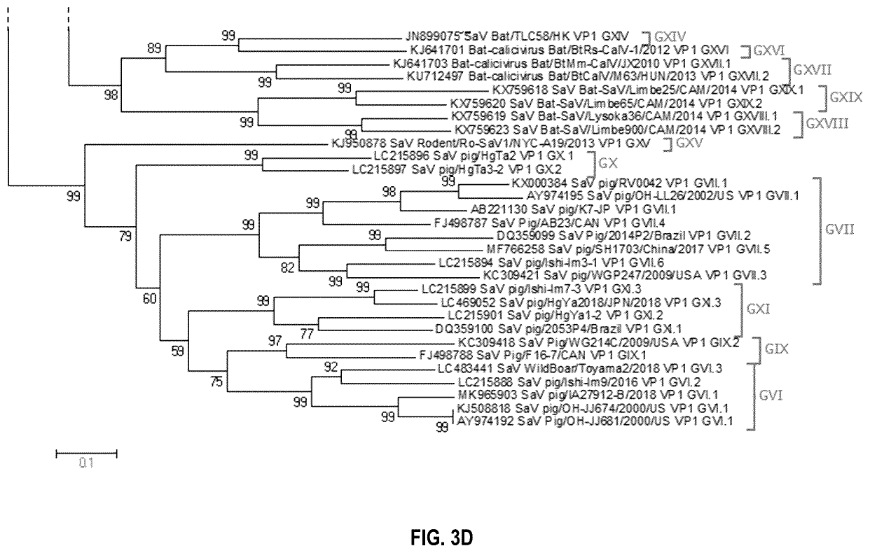

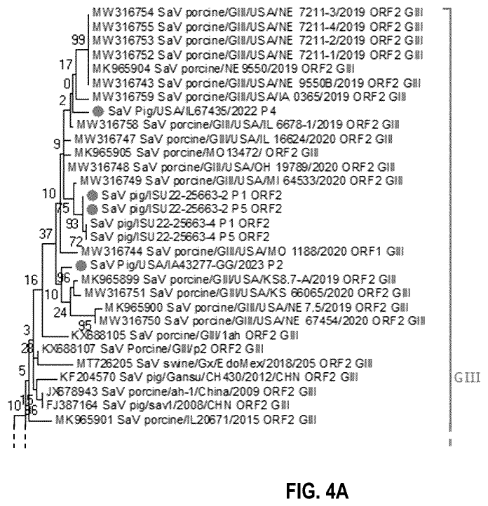

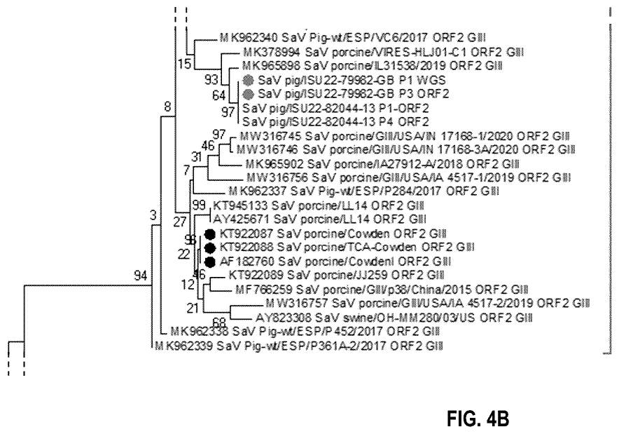

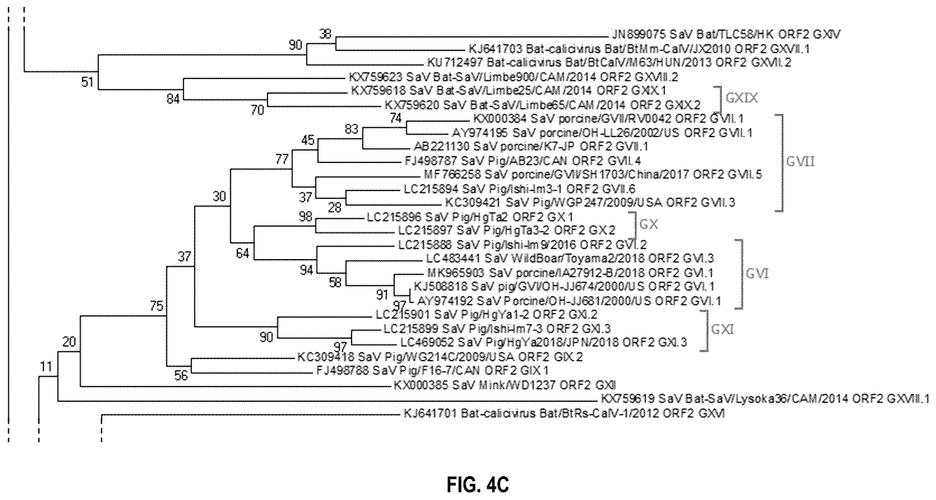

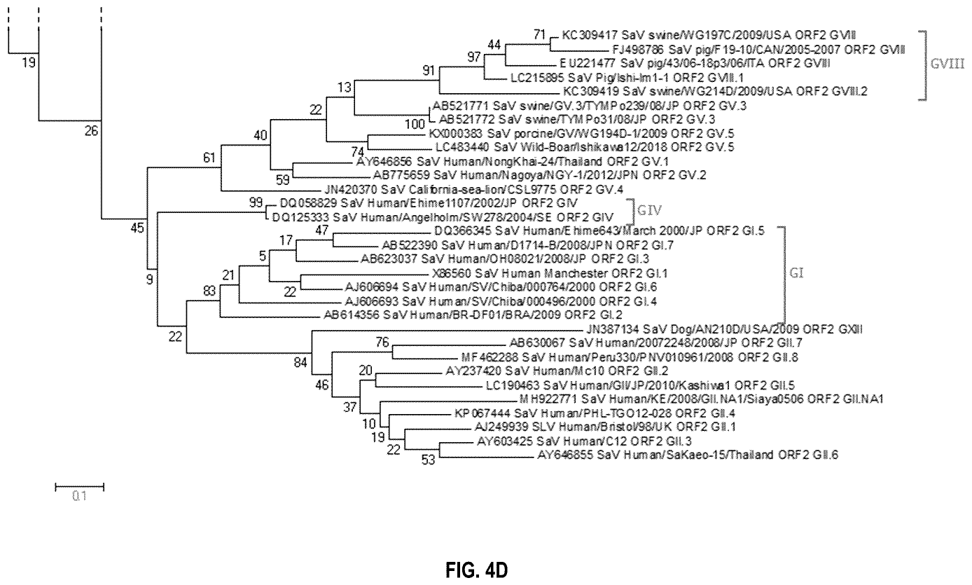

Phylogenetic analysis based on the nearly full-length genome sequences (FIG. 2), the VP1 nucleotide sequences (FIG. 3), and the VP2 nucleotide sequences (FIG. 4) of SaV isolates together with the reference sequences representing 19 genogroups of SaVs confirmed that IL25563-2/2022, IA79982-GB/2022, IL67435-GA/2022, and IA43277-GG/2023 isolates belong to genogroup III.

In summary, four contemporary porcine SaV GIII isolates were obtained from pig samples collected in the USA in 2022 or 2023. The data demonstrated that the porcine SaV isolates are genetically stable after a few serial passages in cell culture. Availability of the contemporary porcine SaV isolates provides an important tool for pathogenesis study, virological and serological assay development, and vaccine development.

### Example 2: Further Serial Passages of the Four Porcine SaV Isolates

The four selected isolates were further characterized. This included serial propagation of each isolate in LLC-PK1 cells for 10 passages (P0-P9). The virus isolates at each passage were tested by porcine SaV real-time RT-PCR assay to determine the Ct values and were titrated for infectious titers expressed as the median tissue culture infectious dose per milliliter (TCID50/ml). Whole genome sequences of the porcine SaV isolates at P9 were determined using next generation sequencing (NGS) technology.

The nucleotide identities between these SaV isolates at the whole genome level, VP1 genomic region, and VP2 genomic regions are summarized in Tables 4-6. Table 4 shows the nucleotide identity of four contemporary porcine sapovirus GIII isolates and the prototype GIII Cowden isolate at the whole genome level. Table 5 shows the nucleotide identity of four contemporary porcine sapovirus GIII isolates and the prototype GIII Cowden isolate at the VP1 nucleotide level. Table 6 shows the nucleotide identity of four contemporary porcine sapovirus GIII isolates and the prototype GIII Cowden isolate at the VP2 nucleotide level.

TABLE 4

| | AF182760_{—} | SaV_pig/ | SaV_pig/ | SaV_pig/ | SaV_pig/ |
|---|---|---|---|---|---|
| | SaV_pig/ | IL25663- | IL25663- | IL25663- | IA79982- |
| | Cowden | 2/2022_P1 | 2/2022_P5 | 2/2022_P9 | GB/2022_P1 |
| AF182760_SaV_{—} | 100.00% | 83.56% | 83.56% | 83.57% | 83.49% |
| pig/Cowden | | | | | |
| SaV_pig/IL25663- | | 100.00% | 100.00% | 99.99% | 89.63% |
| 2/2022_P1 | | | | | |
| SaV_pig/IL25663- | | | 100.00% | 99.99% | 89.63% |
| 2/2022_P5 | | | | | |
| SaV_pig/IL25663- | | | | 100.00% | 89.61% |
| 2/2022_P9 | | | | | |
| SaV_pig/IA79982- | | | | | 100.00% |
| GB/2022_P1 | | | | | |
| SaV_pig/IA79982- | | | | | |
| GB/2022_P3 | | | | | |
| SaV_pig/IA79982- | | | | | |
| GB/2022_P9 | | | | | |
| SaV_pig/IL67435- | | | | | |
| GA/2022_P4 | | | | | |
| SaV_pig/IL67435- | | | | | |
| GA/2022_P9 | | | | | |
| SaV_pig/IA43277- | | | | | |
| GG/2023_P2 | | | | | |
| SaV_pig/IA43277- | | | | | |
| GG/2023_P9 | | | | | |

| | SaV_pig/ | SaV_pig/ | SaV_pig/ | SaV_pig/ |
|---|---|---|---|---|
| | IA79982- | IA79982- | IL67435- | IL67435- |
| | GB/2022_P3 | GB/2022_P9 | GA/2022_P4 | GA/2022_ [symbol] |
| AF182760_SaV_{—} | 83.49% | 83.41% | 83.76% | 83.76% |
| pig/Cowden | | | | |
| SaV_pig/IL25663- | 89.63% | 89.54% | 93.14% | 93.13% |
| 2/2022_P1 | | | | |
| SaV_pig/IL25663- | 89.63% | 89.54% | 93.14% | 93.13% |
| 2/2022_P5 | | | | |
| SaV_pig/IL25663- | 89.61% | 89.53% | 93.13% | 93.11% |
| 2/2022_P9 | | | | |
| SaV_pig/IA79982- | 100.00% | 99.92% | 91.25% | 91.28% |
| GB/2022_P1 | | | | |
| SaV_pig/IA79982- | 100.00% | 99.92% | 91.25% | 91.28% |
| GB/2022_P3 | | | | |
| SaV_pig/IA79982- | | 100.00% | 91.17% | 91.20% |
| GB/2022_P9 | | | | |
| SaV_pig/IL67435- | | | 100.00% | 99.97% |
| GA/2022_P4 | | | | |
| SaV_pig/IL67435- | | | | 100.00% |
| GA/2022_P9 | | | | |
| SaV_pig/IA43277- | | | | |
| GG/2023_P2 | | | | |
| SaV_pig/IA43277- | | | | |
| GG/2023_P9 | | | | |

[symbol]

indicates data missing or illegible when filed

TABLE 5

| | AF182760_{—} | SaV_pig/ | SaV_pig/ | SaV_pig/ | SaV_pig/ |
|---|---|---|---|---|---|
| | SaV_pig/ | IL25663- | IL25663- | IL25663- | IA79982- |
| | Cowden | 2/2022_P1 | 2/2022_P5 | 2/2022_P9 | GB/2022_P1 |
| AF182760_SaV_{—} | 100.00% | 85.06% | 85.06% | 85.06% | 84.08% |
| pig/Cowden | | | | | |
| SaV_pig/IL25663- | | 100.00% | 100.00% | 100.00% | 81.26% |
| 2/2022_P1 | | | | | |
| SaV_pig/IL25663- | | | 100.00% | 100.00% | 81.26% |
| 2/2022_P5 | | | | | |
| SaV_pig/IL25663- | | | | 100.00% | 81.26% |
| 2/2022_P9 | | | | | |
| SaV_pig/IA79982- | | | | | 100.00% |
| GB/2022_P1 | | | | | |
| SaV_pig/IA79982- | | | | | |
| GB/2022_P3 | | | | | |
| SaV_pig/IA79982- | | | | | |
| GB/2022_P9 | | | | | |
| SaV_pig/IL67435- | | | | | |
| GA/2022_P4 | | | | | |
| SaV_pig/IL67435- | | | | | |
| GA/2022_P9 | | | | | |
| SaV_pig/IA43277- | | | | | |
| GG/2023_P2 | | | | | |
| SaV_pig/IA43277- | | | | | |
| GG/2023_P9 | | | | | |

| | SaV_pig/ | SaV_pig/ | SaV_pig/ | SaV_pig/ |
|---|---|---|---|---|
| | IA79982- | IA79982- | IL67435- | IL67435- |
| | GB/2022_P3 | GB/2022_P9 | GA/2022_P4 | GA/2022_ [symbol] |
| AF182760_SaV_{—} | 84.08% | 83.96% | 84.63% | 84.63% |
| pig/Cowden | | | | |
| SaV_pig/IL25663- | 81.26% | 81.14% | 93.45% | 93.45% |
| 2/2022_P1 | | | | |
| SaV_pig/IL25663- | 81.26% | 81.14% | 93.45% | 93.45% |
| 2/2022_P5 | | | | |
| SaV_pig/IL25663- | 81.26% | 81.14% | 93.45% | 93.45% |
| 2/2022_P9 | | | | |
| SaV_pig/IA79982- | 100.00% | 99.88% | [symbol] | [symbol] |
| GB/2022_P1 | | | | |
| SaV_pig/IA79982- | 100.00% | 99.88% | [symbol] | [symbol] |
| GB/2022_P3 | | | | |
| SaV_pig/IA79982- | | 100.00% | 81.57% | 81.57% |
| GB/2022_P9 | | | | |
| SaV_pig/IL67435- | | | 100.00% | 100.00% |
| GA/2022_P4 | | | | |
| SaV_pig/IL67435- | | | | 100.00% |
| GA/2022_P9 | | | | |
| SaV_pig/IA43277- | | | | |
| GG/2023_P2 | | | | |
| SaV_pig/IA43277- | | | | |
| GG/2023_P9 | | | | |

[symbol]

indicates data missing or illegible when filed

TABLE 6

| | AF182760_{—} | SaV_pig/ | SaV_pig/ | SaV_pig/ | SaV_pig/ |
|---|---|---|---|---|---|
| | SaV_pig/ | IL25663- | IL25663- | IL25663- | IA79982- |
| | Cowden | 2/2022_P1 | 2/2022_P5 | 2/2022_P9 | GB/2022_P1 |
| AF182760_SaV_{—} | 100.00% | 86.46% | 86.46% | 86.46% | 86.06% |
| pig/Cowden | | | | | |
| SaV_pig/IL25663- | | 100.00% | 100.00% | 100.00% | 85.86% |
| 2/2022_P1 | | | | | |
| SaV_pig/IL25663- | | | 100.00% | 100.00% | 85.86% |
| 2/2022_P5 | | | | | |
| SaV_pig/IL25663- | | | | 100.00% | 85.86% |
| 2/2022_P9 | | | | | |
| SaV_pig/IA79982- | | | | | 100.00% |
| GB/2022_P1 | | | | | |
| SaV_pig/IA79982- | | | | | |
| GB/2022_P3 | | | | | |
| SaV_pig/IA79982- | | | | | |
| GB/2022_P9 | | | | | |
| SaV_pig/IL67435- | | | | | |
| GA/2022_P4 | | | | | |
| SaV_pig/IL67435- | | | | | |
| GA/2022_P9 | | | | | |
| SaV_pig/IA43277- | | | | | |
| GG/2023_P2 | | | | | |
| SaV_pig/IA43277- | | | | | |
| GG/2023_P9 | | | | | |

| | SaV_pig/ | SaV_pig/ | SaV_pig/ | SaV_pig/ |
|---|---|---|---|---|
| | IA79982- | IA79982- | IL67435- | IL67435- |
| | GB/2022_P3 | GB/2022_P9 | GA/2022_P4 | GA/2022_ [symbol] |
| AF182760_SaV_{—} | 86.06% | 86.06% | 86.06% | 86.06% |
| pig/Cowden | | | | |
| SaV_pig/IL25663- | 85.86% | 85.86% | 91.92% | 91.92% |
| 2/2022_P1 | | | | |
| SaV_pig/IL25663- | 85.86% | 85.86% | 91.92% | 91.92% |
| 2/2022_P5 | | | | |
| SaV_pig/IL25663- | 85.86% | 85.86% | 91.92% | 91.92% |
| 2/2022_P9 | | | | |
| SaV_pig/IA79982- | 100.00% | 100.00% | 85.66% | 85.66% |
| GB/2022_P1 | | | | |
| SaV_pig/IA79982- | 100.00% | 100.00% | 85.66% | 85.66% |
| GB/2022_P3 | | | | |
| SaV_pig/IA79982- | | 100.00% | 85.66% | 85.66% |
| GB/2022_P9 | | | | |
| SaV_pig/IL67435- | | | 100.00% | 100.00% |
| GA/2022_P4 | | | | |
| SaV_pig/IL67435- | | | | 100.00% |
| GA/2022_P9 | | | | |
| SaV_pig/IA43277- | | | | |
| GG/2023_P2 | | | | |
| SaV_pig/IA43277- | | | | |
| GG/2023_P9 | | | | |

[symbol]

indicates data missing or illegible when filed

### Example 3: Generation and Application of Porcine SaV VP1 Peptide-Specific Antisera

Two porcine SaV GIII VP1 peptides (VP1 peptide OBS-45:63: DPSAVDQPGAFPHALVDAR (SEQ ID NO: 37) and VP1 peptide OBS:21-40: RSLLPRTARTLRGNRFGRPI (SEQ ID NO: 38)) were synthesized and used to immunize rabbits (two rabbits for each peptide) for a duration of 90 days to generate peptide-specific antisera. The VP1 peptide-specific rabbit antisera were tested for their utilization in immunofluorescence staining to verify the virus isolation and titration outcomes.

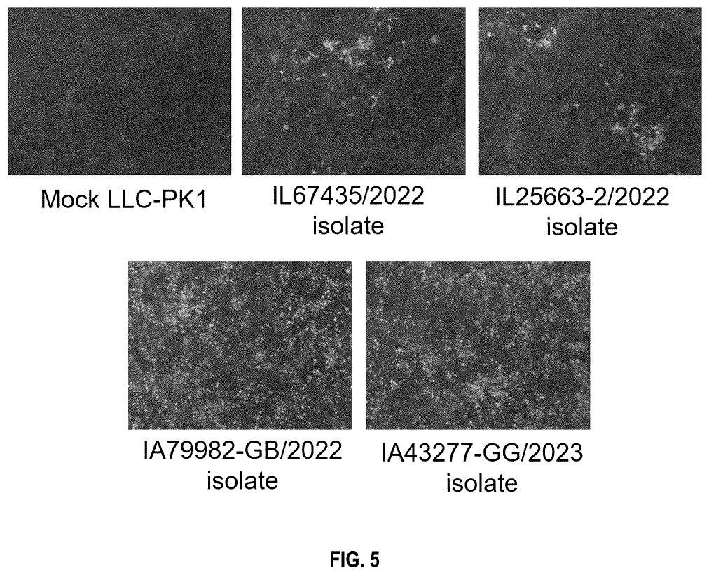

Among four immunized rabbits, VP1 peptide-specific antisera were successfully generated in three rabbits. Immunofluorescence staining of virus-infected cell culture demonstrated that the antisera from the three rabbits were able to stain all of the four contemporary isolates evaluated in the present disclosure (FIG. 5). One antiserum was used to develop porcine SaV IHC assay; strong and clear IHC staining was observed in SaV-positive small intestine tissues (FIG. 6).

### Example 4: Development of Porcine SaV IFA Antibody Assay

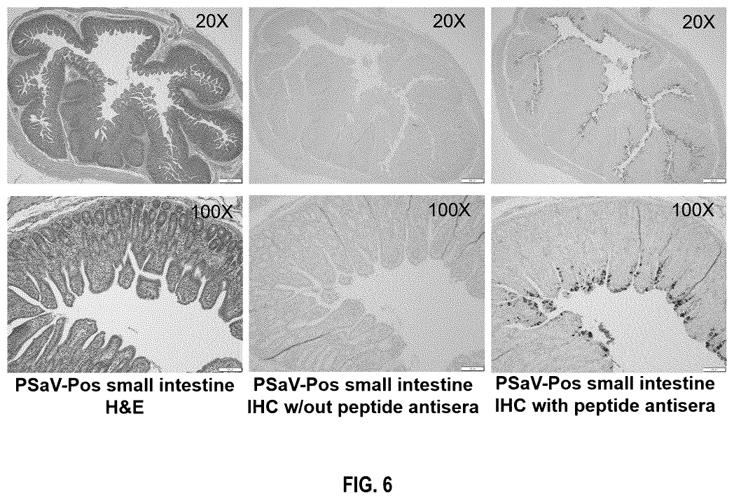

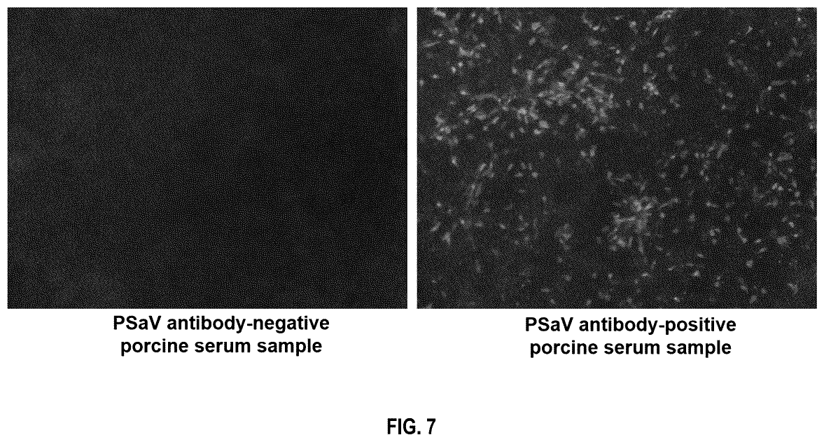

One contemporary porcine SaV isolate was used to develop an indirect fluorescent antibody (IFA) assay for detecting antibody in serum samples (FIG. 7), which was further evaluated using serum samples collected from previous experimental pig studies. Among 87 pigs from Experimental study 1 in 2018, the porcine SaV GIII IFA antibody positive rates were 0% (0/29), 62.1% (18/29), and 96.5% (28/29) in 3-week-old, 8-week-old, and 23-week-old pigs, respectively. For 56 pigs (10 days of age) from Experimental study 2 in 2019, the porcine SaV GIII IFA antibody positive rates were 85.7% (48/56). For 91 pigs (3 weeks of age) from Experimental study 3 in 2022 and 55 pigs (3 weeks of age) from Experimental study 4 in 2022, the porcine SaV GIII IFA antibody positive rates were 27.5% (25/91) and 65.4% (36/55), respectively. Notably, even in pigs categorized as having “high health” status for experimental studies, SaV antibody was detected from a considerable proportion of them.

The contemporary porcine SaV isolates, the VP1 peptide-specific rabbit antisera and the diagnostic assays offer a valuable resource for further characterizing virus replication, sensitivity of the virus to disinfectants and other environmental factors, and viral pathogenicity, and for advancing vaccine development efforts.

### Example 5: Characterization of Pathogenicity and Cross-Protection of Contemporary Porcine Sapovirus Isolates in Weaned Pigs

Little is known about the pathogenicity and immune response to contemporary porcine sapovirus (PSaV) GIII strains due to limited availability of cultured isolates. This study aimed to evaluate the pathogenicity, virus shedding, immune protection, and tissue tropism of two newly isolated PSaV GIII strains (IL25663/2022 and IA79982/2022) in weaned pigs under controlled conditions. Forty-five PSaV naïve pigs (3-4 weeks old) were randomly assigned to three groups (n=15/group): G1 (Neg/IL25663), G2 (IA79982/IL25663), and G3 (IL25663/IL25663). Pigs were inoculated at 0 days post-inoculation (DPI) and challenged with IL25663 at 35 DPI. Five pigs from each group were euthanized at 4 DPI for characterizing pathogenicity and all remaining pigs were euthanized at 42 DPI. Results showed that both contemporary isolates caused detectable infection with viral shedding lasting approximately 14-21 days post inoculation. IL25663 induced higher levels of viral shedding than IA79982 based on rectal swab and oral fluid PCR data. Immunohistochemistry (IHC) at 4 DPI revealed differences in intestinal tissue staining and lesion severity, suggesting a difference in virulence between the two strains. Upon challenge with IL25663, prior exposure to the homologous strain (IL25663) resulted in the greatest reduction in viral shedding, followed by IA79982, and then the naïve group. These findings indicate partial cross-protection but more robust immunity was observed in the homologous challenge. This study provides the first controlled in vivo characterization of contemporary PSaV GIII isolates in swine. Our findings support the existence of strain-specific differences in pathogenicity and immunity, highlighting the need for continued surveillance, diagnostics, and potential vaccine development targeting PSaV genogroup III. Ongoing antibody response evaluations, including IFA and virus neutralization assays, will further clarify the nature and degree of protective immunity.

### INTRODUCTION

Sapoviruses (SaVs) are members of the Sapovirus genus in the Caliciviridae family (Vinje et al., 2019). Sapoviruses possess a single-stranded, positive-sense RNA genome of 7-8 kb in length, which is composed of two overlapping open reading frames (ORFs): ORF1 and ORF2. ORF1 encodes the nonstructural proteins and the capsid protein VP1 while ORF2 encodes the minor structural protein VP2 (Guo et al., 1999). Sapoviruses are highly genetically diverse and have been classified into 20 genogroups based on the VP1 sequences (GI to GXX) (Nagai et al., 2020; Oka et al., 2016; Van Brussel et al., 2022). Sapoviruses mainly cause enteric infections and have been detected in humans and a variety of animal species (e.g., pig, mink, dog, sea lion, bat, chimpanzee, rat, and so on) (Katsuta et al., 2019; Matamoros et al., 2023; Nagai et al., 2020; Oka et al., 2016). SaVs in genogroup V have been identified in humans, pigs, and sea lions, suggesting the zoonotic potential of GV SaVs. Thus far, eight genogroups of SaVs (GIII and GV-GXI) have been detected in pigs with GIII being most commonly detected (Kuroda et al., 2017; Martella et al., 2008; Nagai et al., 2020). For porcine sapoviruses, the historical Cowden strain was isolated in cell culture in 1980s in the United States (Chang et al., 2004; Flynn and Saif, 1988; Parwani et al., 1991) and very few contemporary isolates have been obtained. Our laboratory successfully obtained several contemporary porcine SaV isolates in cell culture in 2022 (Aljets et al., unpublished).

Little is known about the pathogenicity and immune response to contemporary PSaV GIII strains. In this study, we aimed to characterize the pathogenicity, virus shedding, immune protection, and tissue tropism of two newly isolated PSaV GIII strains (IL25663/2022 and IA79982/2022) in comparison with the historical Cowden strain in weaned pigs under controlled conditions.

### Materials and Methods

### Cells and Virus Isolates

The LLC-PK1 (ATCC CCL-101) cells were cultured in MEM supplemented with 10% fetal bovine serum, 1% L-glutamine, 1% non-essential amino acids (NEAA), and 1× antibiotics (0.05 mg/ml gentamicin, 100 Unit/mL penicillin, 100 μg/mL streptomycin, and 0.25 μg/mL amphotericin). Two contemporary porcine SaV isolates (USA/IL25663/2022 and USA/IA79982/2022) were previously obtained in our laboratory (Aljets et al., unpublished). The porcine SaV Cowden isolate was obtained from Dr. Linda Saif at the Ohio State University. Porcine SaV isolates were propagated and titrated in LLC-PK1 cells.

### Animals and Study Design

The study was approved by the Iowa State University Institutional Animal Care and Use Committee (approval number IACUC-24-003) and the Institutional Biosafety Committee (approval number IBC-24-003).

Sixty piglets at 12 days of age were prescreened. Rectal swabs of these piglets were negative for porcine epidemic diarrhea virus (PEDV), porcine deltacoronavirus (PDCoV), transmissible gastroenteritis virus (TGEV), porcine SaV, and porcine rotavirus A (RVA), porcine rotavirus B (RVB), and porcine rotavirus C (RVC) via PCRs. Serum samples of these piglets were confirmed negative for porcine SaV antibody using an in-house developed indirect fluorescent antibody (IFA) assay.

When these 60 piglets were 3 weeks old, they were delivered to the Iowa State University Laboratory Animal Resources (ISU LAR) facility (−7 days post inoculation [DPI]). Sixty pigs were randomly assigned to four groups, with 15 pigs per group per room (Table 7): G1 (Neg/IL25663), G2 (IA79982/IL25663), G3 (IL25663/IL25663), and G4 (Cowden/IL25663). At −7 DPI, each pig was microchipped to monitor body temperature as described previously (Rawal et al., 2023), rectal swabs were collected again for PCR testing for porcine SaV, PEDV, PDCoV, TGEV, and porcine RVA, RVB, and RVC, and serum samples were recollected for porcine SaV IFA antibody testing. At −2 DPI, rectal swabs were collected again for PCR testing for porcine SaV, PEDV, PDCoV, TGEV, and porcine RVA, RVB, and RVC. Since pigs in G4 became positive for porcine sapovirus before 0 day post inoculation (DPI), the entire G4 was excluded from this study (see Results section for detailed information).

At 0 DPI, pigs in G2 and G3 were orally inoculated with 10 ml of the PSaV isolates IL25563/2022 and IA79982/2022 at the titers of 10∝TCID50/ml, respectively (10≡TCID50/pig), while pigs in G1 received the same volume of virus-negative medium as negative control. At 4 DPI, five pigs per group were randomly selected and necropsied to characterize gross and microscopic lesions in enteric tissues. At 35 DPI, all remaining pigs were orally challenged with PSaV isolate IL25563-2/2022 (10\hat{ }5 TCID50/pig). At 42 DPI (7 days post challenge), all remaining pigs were euthanized for necropsy.

Daily microchip temperature was recorded. Pigs were monitored daily for clinical signs, including diarrhea, depression, and body conditions following the previous criteria in PEDV studies (Chen et al., 2016). Pigs were weighed at 0, 4, 7, 10, 14, 21, 28, 35, and 42 DPI. The weight difference between two time points were divided by the number of days in the interval to calculate the average daily weight gain (ADG). Individual rectal swab samples were collected daily during 0-4 DPI, at 7, 10, 14, 21, 28, and 35 DPI, and then daily during 36-42 DPI. Pen-based oral fluid samples (2 ropes per pen at each time point) were collected daily during 0-7 DPI, at 10, 14, 21, 28, and 35 DPI, and then daily during 36-42 DPI. Individual serum samples were collected at 0, 4, 7, 10, 14, 21, 28, 35, and 42 DPI. At necropsy, gross lesions on small intestines (duodenum, jejunum, and ileum), cecum, and colon were scored as previously described for PEDV (Chen et al., 2016). Fresh and formalin-fixed small intestine (duodenum, jejunum, and ileum), cecum, and colon tissues were collected.

### Nucleic Acid Extraction and Porcine Sapovirus Real-Time RT-PCR

Nucleic acids were extracted from samples using a MagMAX Pathogen RNA/DNA Kit (Thermo Fisher Scientific, Waltham, MA, USA) and a Kingfisher Flex instrument (Thermo Fisher Scientific) following the manufacturer's instructions. One hundred microliters of the sample was used for extraction, and nucleic acid was eluted into 90 μL elution buffer.

A previously described porcine sapovirus genogroup III real-time RT-PCR (Aljets et al., unpublished) was used in this study to test for the presence of porcine SaV in samples. Samples with Ct<40 were considered PCR-positive and samples with Ct≥40 were considered PCR-negative for SaV.

A previously described PEDV/PDCoV/TGEV multiplex real-time RT-PCR (Zhu et al., 2022) was used to prescreen pigs. Samples with Ct<40 were considered PCR-positive and samples with Ct≥40 were considered PCR-negative.

A commercial porcine rotaviruses A, B, and C multiplex real-time RT-PCR (VetMAX™ Rotavirus A/B/C Reagents, Thermo Fisher Scientific) was used to test for the presence of porcine RVA, RVB, and RVC in samples. Samples with Ct<36 were considered PCR-positive and samples with Ct≥36 were considered PCR-negative according to the instructions.

### Gross Lesions and Microscopic Lesions

At necropsy, the small intestine, cecum, and colon from each pig were examined in a blind fashion by a single pathologist and given a subjective score for the severity of gross lesions using an established scoring system (Chen et al., 2016). Duodenum, proximal jejunum, middle jejunum, distal jejunum, ileum, cecum, and colon tissues fixed in 10% formalin were embedded, sectioned, and stained with hematoxylin and eosin (H&E) and examined by a veterinary pathologist blinded to individual animal identifications and treatment groups. Villus lengths and crypt depths were measured from three representative villi and crypts of duodenum, proximal jejunum, middle jejunum, distal jejunum, and ileum, using a computerized image system following previously described procedures (Madson et al., 2014). Villus-height-to-crypt-depth (villus/crypt) ratio of each tissue was calculated as the quotient of the average villus length divided by the average crypt depth.

### Immunohistochemistry

Serial sections of duodenum, proximal jejunum, middle jejunum, distal jejunum, ileum, and colon at 4 DPI necropsy were evaluated for porcine SaV antigen by immunohistochemistry (IHC) using PSaV VP1 peptide-specific antisera generated in rabbits as previously described (Aljets et al., unpublished). The same sections of tissues were also evaluated for porcine rotavirus A antigen by IHC using porcine RVA VP6 protein-specific mouse monoclonal antibody. The IHC antigen detection was semi-quantitatively scored as previously described (Chen et al., 2015) with the following criteria: 0=no staining; 1=approximately 1-10% enterocytes with positive staining; 2=approximately 10%-25% enterocytes with positive staining; 3=approximately 25%-50% enterocytes with positive staining; 4=approximately 50%-100% enterocytes with positive staining.

### Porcine Sapovirus Antibody Assays

Serum samples collected at 0, 7, 10, 14, 21, 28, 35, and 42 DPI were tested by IFA antibody assay using porcine SaV isolates IL25663/2022, IA79982/2022, and Cowden as the indicator virus, respectively.

The same set of serum samples were also tested for neutralizing antibodies using porcine SaV isolates IL25663/2022, IA79982/2022, and Cowden as the indicator virus, respectively.

### Statistical Analyses

A linear mixed (GLIMMIX) model with Statistical Analysis System (SAS) will be used to analyze diarrhea and clinical scores, weight, IFA antibody Log₂ titers, neutralizing antibody Log₂ titers, and rectal swab PCR genomic copies over time. One-way ANOVA will be used to analyze average daily weight gain (ADG), PCR genomic copies in enteric tissues, gross lesion scores, microscopic lesion scores, and IHC scores. A p value <0.05 will be considered significantly different.

### Results

### Prescreening Test Results and Additional Test Results Before Inoculation

The results of prescreening testing (−16 DPI) and additional testing conducted before inoculation (−7 DPI and −2 DPI) are summarized in Table 8.

At −16 DPI, when piglets were 12 days old and still nursing on sows, rectal swabs of all 60 piglets tested negative for PSaV, PEDV, PDCoV, TGEV, RVA, RVB, and RVC by PCRs. Serum samples from all 60 piglets tested negative for PSaV IFA antibodies.

At −7 DPI, when pigs were delivered to the ISU LAR Animal Facility, individual rectal swabs from all 60 pigs were tested for PSaV by PCR; one pig in G4 (Cowden/IL25663) tested positive for PSaV (Ct=31.9) while the remaining 59 pigs tested negative for PSaV. Serum samples from all 60 pigs tested negative for PSaV IFA antibodies. Sixty rectal swabs were also tested for PEDV, PDCoV, TGEV, RVA, RVB, and RVC in pool of 5 by PCR. A total of 12 pools in four groups all tested negative for PEDV, PDCoV, TGEV, and RVB. In G1, 2/3 pools tested positive for RVA (average Ct=32.7) and 0/3 pool tested positive for RVC; in G2, 3/3 pools tested positive for RVA (average Ct=34.2) and 1/3 pool tested positive for RVC (Ct=34.1); in G3, 3/3 pools tested positive for RVA (average Ct=27.8) and 1/3 pool tested positive for RVC (Ct=23.7); and in G4, 2/3 pools tested positive for RVA (average Ct=31.7) and 0/3 pool tested positive for RVC.

At −2 DPI, 15/15 pigs in G4 tested positive for PSaV by PCR (average Ct=28.8; Ct ranges 17.1-37.3) while all pigs in G1, G2 and G3 tested negative for PSaV by PCR. All pigs in G1 through G4 tested positive for RVA by PCR and negative for RVB by PCR. All pigs in G1 and G4 tested negative for RVC by PCR, while 5/15 pigs in G2 and 15/15 pigs in G3 tested positive for RVC by PCR (Table 8).

Based on these results, we decided to eliminate G4 from the study. For G1, G2, and G3, which were retained for the PSaV study, we had to acknowledge that all pigs in these groups were positive for RVA and that some pigs were positive RVC before inoculation with PSaV. Clinical observations (diarrhea score, microchip temperature, and ADG)

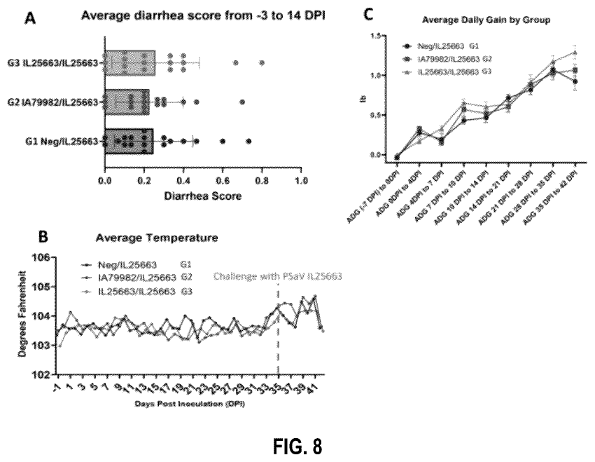

From −3 DPI through 14 DPI, mild to moderate diarrhea was observed in 8/15, 11/15, 14/15 pigs for at least one day in G1, G2 and G3, respectively; the number of pigs with diarrhea lasting for ≥3 days was 7/15, 6/15, and 9/15 pigs in G1, G2 and G3, respectively. The overall average diarrhea scores in different groups from −3 to 14 DPI are shown in FIG. 8A. Upon challenge at 35 DPI, mild diarrhea was observed in two pigs of G1 (Neg/IL25663) at 37 DPI and had cleared at 40 DPI. Depression was observed in a few pigs in each group during the first 14 days after arrival, this appeared to be typical post-wean lag.

The microchip temperature of pigs slightly fluctuated between −1 DPI and 35 DPI but mostly fell within the normal range (≤104° F. or ≤40° C.). After challenge with PSaV IL25663/2022 strain on 35 DPI, G1 and G2 pigs had numerical higher temperature than G3 pigs at some time points (FIG. 8B) but statistical analysis remains to be done.

The mean average daily weight gain (ADG) data between different time intervals are summarized in FIG. 8C. ADG in G3 appeared to be higher than that in G1 and G2 from 35 DPI to 42 DPI but statistical analysis remains to be done.

### Shedding of Porcine Rotavirus A and Rotavirus C in Rectal Swabs

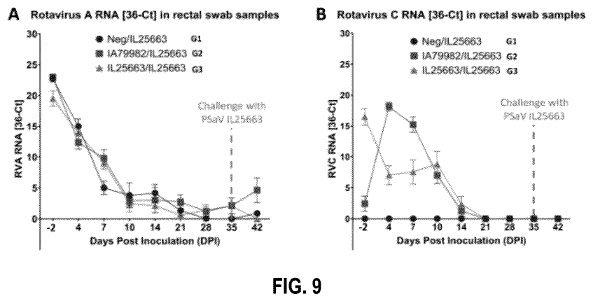

At −2 DPI, all pigs in G1, G2 and G3 were already porcine RVA PCR positive with similar viral RNA loads in rectal swab samples (FIG. 9A). The RVA RNA loads continued to decrease over time and reached a low level at 10 DPI and thereafter, with similar patterns across G1, G2 and G3 (FIG. 9A). The number of RVA PCR-positive pigs in each group is summarized in Table 9. At 4 DPI, 15/15 pigs in each group were still RVA PCR positive. At 35 DPI and 42 DPI, RVA PCR positivity was 0/10, 4/10, and 3/10, and 4/10, 8/10, and 2/10 pigs in G1, G2, and G3, respectively, with low viral loads at both time points (Table 9 and FIG. 9A).

Porcine RVC was consistently PCR negative in rectal swab samples from G1 throughout the study. In contrast, RVC was detected by PCR in G2 and G3 pigs, showing distinct temporal patterns. At −2 DPI, 5/15 pigs in G2 and all 15 pigs in G3 tested RVC PCR positive, with G3 exhibiting a higher viral RNA load than G2 (FIG. 9B). In G2, the RVC RNA load increased to a peak at 4 DPI, then gradually declined, becoming undetectable at 21 DPI and thereafter. In G3, the RVC RNA load decreased steadily after −2 DPI and reached undetectable at 21 DPI and thereafter (FIG. 9B). The number of RVC PCR-positive pigs in each group is summarized in Table 9. At 4 DPI, all pigs in both G2 and G3 (15/15) were RVC PCR positive, whereas both groups tested RVC PCR negative at 21 DPI and remained negative through 42 DPI (Table 9).

### Shedding of Porcine Sapovirus in Rectal Swabs and Oral Fluids

All pigs in G1, G2 and G3 tested negative for porcine sapovirus by PCR at −16, −7, −2 and 0 DPI and by IFA antibody assay at −16 DPI and −7 DPI (Table 8 and Table 9).

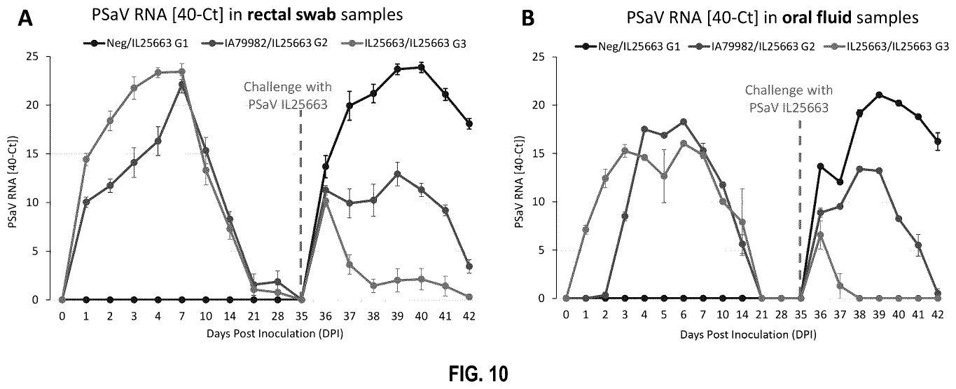

All G1 pigs remained PSaV PCR negative in rectal swabs through 35 DPI (Table 9). In both G2 and G3, all pigs became PSaV PCR positive in rectal swabs at 1 DPI and remained positive through 14 DPI. At 21 DPI, 3/10 pigs in G2 and 2/10 pigs in G3 tested PSaV PCR positive, but at 35 DPI, all pigs in both groups became undetectable by PSaV PCR (Table 9). After challenge with the PSaV IL25663/2022 strain at 35 DPI, all pigs in G1 (Neg/IL25663) and G2 (IA79982/IL25663) were PSaV PCR positive from 36 to 42 DPI, whereas the number of PSaV PCR-positive pigs in G3 (IL25663/IL25663) was 10/10 at 36 DPI, declining to 2/10 at 41 and 42 DPI (Table 9). Regarding the PSaV RNA loads in rectal swabs, G3 pigs inoculated with PSaV IL25663/2022 strain had a higher level than G2 pigs inoculated with PSaV IA79982/2022 strain during 1-6 DPI (FIG. 10A). From 7 to 35 DPI, the two PSaV strains in G2 and G3 had similar viral loads. After challenge with the PSaV IL25663/2022 strain at 35 DPI, the viral loads in rectal swabs were lower in G2 and G3 than in G1 (FIG. 10A), indicating that prior exposure to PSaV reduced shedding of the challenge virus. Furthermore, viral loads in rectal swabs of G3 pigs were lower than those in G2 pigs, suggesting that prior exposure to a PSaV strain homologous to the challenge strain conferred greater reduction in viral shedding than exposure to heterologous virus strain (FIG. 10A).

In oral fluid samples, the onset of viral shedding occurred earlier in G3 pigs than in G2 pigs, and the viral loads were higher in G3 than in G2 from 1 to 3 DPI (FIG. 10B). From 4 to 6 DPI, viral loads in the oral fluids of G2 pigs were numerically higher than those in G3 pigs. From 7 to 14 DPI, no distinct differences in viral loads were observed in oral fluids between G2 and G3 pigs (FIG. 10B). All pigs in G2 and G3 became undetectable for PSaV by PCR in oral fluids during 21-35 DPI (FIG. 10B). Following challenge with the PSaV IL25663/2022 strain at 35 DPI, viral loads in oral fluid samples were highest in G1, followed by G2 and G3, showing a pattern similar to that observed in rectal swabs (FIG. 10B).

### Gross Lesions, Histopathological Lesions, and Immunohistochemistry Staining

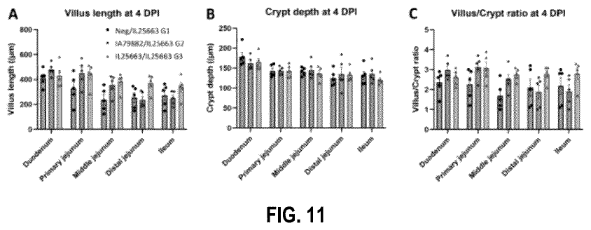

Villus length, crypt depth, and villus length/crypt depth ratio of duodenum, primary jejunum, middle jejunum, distal jejunum, and ileum tissues from pigs necropsied at 4 DPI are presented in FIG. 11.

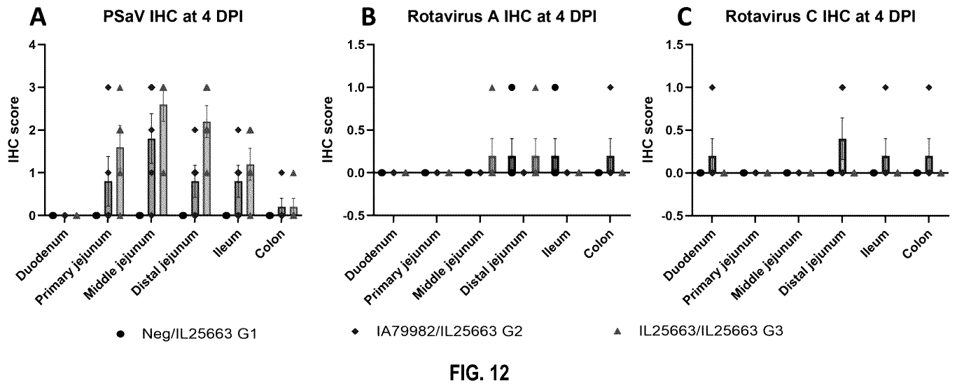

PSaV IHC staining data on tissues from pigs necropsied at 4 DPI are shown in FIG. 12A. No PSaV IHC staining was observed in duodenum regardless of the virus strains. Very low level of PSaV IHC staining was observed in colon tissues of G2 and G3 pigs. PSaV IHC staining was evidently observed in primary jejunum, middle jejunum, distal jejunum, and ileum tissues, with numerically higher IHC scores in G3 (IL25663) pigs than in G2 (IA79882) pigs.

Although RVA was detected in all pigs in G1, G2 and G3 by PCR, RVA antigen was not consistently detected by IHC in enteric tissues at 4 DPI (FIG. 12B).

Very low level of rotavirus C IHC staining was observed in some small intestine tissues of G2 pigs (FIG. 12C).

### DISCUSSION

In the literature, the pathogenicity of porcine sapovirus was only characterized using the historical Cowden isolate (Guo et al., 2001). The pathogenicity and immune responses of contemporary PSaV isolates in pigs have not been previously reported. In this study, we aimed to characterize the pathogenicity and antibody responses of two contemporary PSaV isolates (IA79982/2022 and IL25663/2022) in comparison with the Cowden isolate.

Although piglets were prescreened and confirmed negative for PSaV, PEDV, PDCoV, TGEV, RVA, RVB, and RVC at 12 days of age (−16 DPI), one pig tested PSaV PCR positive at −7 DPI upon arrival at the ISU Animal Facility. This pig apparently transmitted the virus to other pigs in G4 (Cowden/IL25663), resulting in all 15 pigs in the group testing PSaV PCR positive at −2 DPI. Consequently, the G4 pigs were excluded from the study and were not inoculated with the Cowden strain at 0 DPI. As a result, comparison of pathogenicity and antibody responses between the contemporary PSaV isolates and the Cowden isolate could not be performed. Another unexpected thing was that all pigs in G1, G2 and G3 tested RVA PCR positive at −2 DPI and some pigs in G2 and G3 were also RVC PCR positive at −2 DPI. Despite the preexisting rotavirus infection, PSaV inoculation was carried out as scheduled at 0 DPI.

Interpretations of clinical signs, gross lesions in enteric tissues, and microscopic lesions in enteric tissues were complicated by preexisting rotavirus infection. Nonetheless, PSaV-specific effects on shedding dynamics, IHC scores, and protection upon challenge at 35 DPI were observable.

Results showed that both contemporary PSaV isolates caused detectable infection with viral shedding lasting <35 days post inoculation in rectal swabs and <21 days post inoculation in oral fluid samples. The PSaV isolate IL25663/2022 induced higher levels of viral shedding than the IA79982/2022 isolate based on rectal swab PCR data. PSaV IHC at 4 DPI revealed differences in intestinal tissue staining and lesion severity, suggesting a difference in virulence between the two PSaV strains. Upon challenge with the PSaV IL25663/2022 strain, prior exposure to the homologous strain (IL25663/2022) resulted in the greatest reduction in viral shedding, followed by the IA79982/2022 strain, and then the negative/challenge group. These findings indicate partial cross-protection but more robust immunity was observed in the homologous challenge.

This study provides the first controlled in vivo characterization of contemporary PSaV GIII isolates in swine. Our findings support the existence of strain-specific differences in pathogenicity and immunity, highlighting the need for continued surveillance, diagnostics, and potential vaccine development targeting PSaV genogroup III. Ongoing antibody response evaluations, including IFA and virus neutralization assays, will further clarify the nature and degree of protective immunity.

### REFERENCES

Chang, K. O., Sosnovtsev, S. V., Belliot, G., Kim, Y., Saif, L. J., Green, K. Y., 2004. Bile acids are essential for porcine enteric calicivirus replication in association with down-regulation of signal transducer and activator of transcription 1. Proc Natl Acad Sci USA 101, 8733-8738.
Chen, Q., Gauger, P., Stafne, M., Thomas, J., Arruda, P., Burrough, E., Madson, D., Brodie, J., Magstadt, D., Derscheid, R., Welch, M., Zhang, J., 2015. Pathogenicity and pathogenesis of a United States porcine deltacoronavirus cell culture isolate in 5-day-old neonatal piglets. Virology 482, 51-59.
Chen, Q., Gauger, P. C., Stafne, M. R., Thomas, J. T., Madson, D. M., Huang, H., Zheng, Y., Li, G., Zhang, J., 2016. Pathogenesis comparison between the United States porcine epidemic diarrhoea virus prototype and S-INDEL-variant strains in conventional neonatal piglets. J Gen Virol 97, 1107-1121.
Flynn, W. T., Saif, L. J., 1988. Serial propagation of porcine enteric calicivirus-like virus in primary porcine kidney cell cultures. J Clin Microbiol 26, 206-212.
Guo, M., Chang, K. O., Hardy, M. E., Zhang, Q., Parwani, A. V., Saif, L. J., 1999. Molecular characterization of a porcine enteric calicivirus genetically related to Sapporo-like human caliciviruses. J Virol 73, 9625-9631.
Guo, M., Hayes, J., Cho, K. O., Parwani, A. V., Lucas, L. M., Saif, L. J., 2001. Comparative pathogenesis of tissue culture-adapted and wild-type Cowden porcine enteric calicivirus (PEC) in gnotobiotic pigs and induction of diarrhea by intravenous inoculation of wild-type PEC. J Virol 75, 9239-9251.
Katsuta, R., Sunaga, F., Oi, T., Doan, Y. H., Tsuzuku, S., Suzuki, Y., Sano, K., Katayama, Y., Omatsu, T., Oba, M., Furuya, T., Ouchi, Y., Shirai, J., Mizutani, T., Oka, T., Nagai, M., 2019. First identification of Sapoviruses in wild boar. Virus Res 271, 197680.
Kuroda, M., Masuda, T., Ito, M., Naoi, Y., Doan, Y. H., Haga, K., Tsuchiaka, S., Kishimoto, M., Sano, K., Omatsu, T., Katayama, Y., Oba, M., Aoki, H., Ichimaru, T., Sunaga, F., Mukono, I., Yamasato, H., Shirai, J., Katayama, K., Mizutani, T., Oka, T., Nagai, M., 2017. Genetic diversity and intergenogroup recombination events of sapoviruses detected from feces of pigs in Japan. Infect Genet Evol 55, 209-217.
Madson, D. M., Magstadt, D. R., Arruda, P. H., Hoang, H., Sun, D., Bower, L. P., Bhandari, M., Burrough, E. R., Gauger, P. C., Pillatzki, A. E., Stevenson, G. W., Wilberts, B. L., Brodie, J., Harmon, K. M., Wang, C., Main, R. G., Zhang, J., Yoon, K. J., 2014. Pathogenesis of porcine epidemic diarrhea virus isolate (US/Iowa/18984/2013) in 3-week-old weaned pigs. Vet Microbiol 174, 60-68.
Martella, V., Banyai, K., Lorusso, E., Bellacicco, A. L., Decaro, N., Mari, V., Saif, L., Costantini, V., De Grazia, S., Pezzotti, G., Lavazza, A., Buonavoglia, C., 2008. Genetic heterogeneity of porcine enteric caliciviruses identified from diarrhoeic piglets. Virus Genes 36, 365-373.
Matamoros, D. J. P., Worsfold, C. S., Campos, R. C., Acuna, H. M. B., Chacon, E. C., Sanchez, C. F. J., 2023. Molecular characterization of norovirus and sapovirus detected in animals and humans in Costa Rica: Zoo-anthropozoonotic potential of human norovirus GII.4. Open Vet J 13, 74-89.
Nagai, M., Wang, Q., Oka, T., Saif, L. J., 2020. Porcine sapoviruses: Pathogenesis, epidemiology, genetic diversity, and diagnosis. Virus Res 286, 198025.
Oka, T., Lu, Z., Phan, T., Delwart, E. L., Saif, L. J., Wang, Q., 2016. Genetic Characterization and Classification of Human and Animal Sapoviruses. PLoS One 11, e0156373.
Parwani, A. V., Flynn, W. T., Gadfield, K. L., Saif, L. J., 1991. Serial propagation of porcine enteric calicivirus in a continuous cell line. Effect of medium supplementation with intestinal contents or enzymes. Arch Virol 120, 115-122.
Rawal, G., Almeida, M. N., Gauger, P. C., Zimmerman, J. J., Ye, F., Rademacher, C. J., Armenta Leyva, B., Munguia-Ramirez, B., Tarasiuk, G., Schumacher, L. L., Aljets, E. K., Thomas, J. T., Zhu, J. H., Trexel, J. B., Zhang, J., 2023. In Vivo and In Vitro Characterization of the Recently Emergent PRRSV 1-4-4 LlC Variant (L1C.5) in Comparison with Other PRRSV-2 Lineage 1 Isolates. Viruses 15, 2233.
Van Brussel, K., Mahar, J. E., Ortiz-Baez, A. S., Carrai, M., Spielman, D., Boardman, W. S. J., Baker, M. L., Beatty, J. A., Geoghegan, J. L., Barrs, V. R., Holmes, E. C., 2022. Faecal virome of the Australian grey-headed flying fox from urban/suburban environments contains novel coronaviruses, retroviruses and sapoviruses. Virology 576, 42-51.
Vinje, J., Estes, M. K., Esteves, P., Green, K. Y., Katayama, K., Knowles, N.J., L'Homme, Y., Martella, V., Vennema, H., White, P. A., Ictv Report, C., 2019. ICTV Virus Taxonomy Profile: Caliciviridae. J Gen Virol 100, 1469-1470.
Zhu, J. H., Rawal, G., Aljets, E., Yim-Im, W., Yang, Y. L., Huang, Y. W., Krueger, K., Gauger, P., Main, R., Zhang, J., 2022. Development and clinical applications of a 5-plex real-time RT-PCR for swine enteric coronaviruses. Viruses 14, 1536.

TABLE 7
Experimental design of animal study

| Group (number | 0 DPI | | | |
|---|---|---|---|---|
| of pigs) | (Inoculation)* | 4 DPI | 35 DPI* | 42 DPI |
| G1: Neg/IL25663 | Virus-negative | Euthanize and | Challenge all remaining | Euthanize and necropsy |
| (N = 15) | medium; | necropsy 5 | pigs with SaV | all remaining pigs |
| | N = 15 | inoculated pigs | IL25663/2022 isolate | |
| G2: | SaV | Euthanize and | Challenge all remaining | Euthanize and necropsy |
| IA79982/IL25663 | IA79982/2022 | necropsy 5 | pigs with SaV | all remaining pigs |
| (N = 15) | isolate; N = 15 | inoculated pigs | IL25663/2022 isolate | |
| G3: | SaV | Euthanize and | Challenge all remaining | Euthanize and necropsy |
| IL25663/IL25663 | IL25663/2022 | necropsy 5 | pigs with SaV | all remaining pigs |
| (N = 15) | isolate; N = 15 | inoculated pigs | IL25663/2022 isolate | |
| G4: | Eliminated from the study | | | |
| Cowden/IL25663 | | | | |
| (N = 15)† | | | | |

*Pig oral inoculation at 0 DPI: 10\hat{ }5 TCID50/pig; pig oral challenge at 35 DPI: 10\hat{ }5 TCID50/pig.
†Since pigs in G4 became positive for porcine sapovirus before 0 DPI, the entire G4 was eliminated from the study.

TABLE 8
Prescreening test results and additional test results before inoculation with porcine sapoviruses

| Group (number | | | |
|---|---|---|---|
| of pigs) | −16 DPI | −7 DPI | −2 DPI |
| G1: Neg/IL25663 | 0/15 pigs: PSaV PCR Pos | 0/15 pigs: PSaV PCR Pos | 0/15 pigs: PSaV PCR Pos |
| (N = 15) | 0/15 pigs: PEDV/PDCoV/ | 0/3 pools (pool of 5): PEDV/ | 15/15 pigs: RVA PCR Pos |
| | TGEV PCR Pos | PDCoV/TGEV PCR Pos | (average Ct 13.2) |
| | 0/15 pigs: RVA/RVB/RVC | 2/3 pools (pool of 5): RVA | 0/15 pigs: RVB PCR Pos |
| | PCR Pos | PCR Pos (average Ct 32.7) | 0/15 pigs: RVC PCR Pos |
| | 0/15 pigs: PSaV IFA | 0/3 pools (pool of 5): RVB PCR Pos | |
| | antibody Pos | 0/3 pools (pool of 5): RVC PCR Pos | |
| | | 0/15 pigs: PSaV IFA antibody Pos | |
| G2: | 0/15 pigs: PSaV PCR Pos | 0/15 pigs: PSaV PCR Pos | 0/15 pigs: PSaV PCR Pos |
| LA79882/1L25663 | 0/15 pigs: PEDV/PDCoV/ | 0/3 pools (pool of 5): PEDV/ | 15/15 pigs: RVA PCR Pos |
| (N = 15) | TGEV PCR Pos | PDCoV/TGEV PCR Pos | (average Ct 13.0) |
| | 0/15 pigs: RVA/RVB/RVC | 3/3 pools (pool of 5): RVA PCR | 0/15 pigs: RVB PCR Pos |
| | PCR Pos | Pos (average Ct 34.2) | 5/15 pigs: RVC PCR Pos |
| | 0/15 pigs: PSaV IFA | 0/3 pools (pool of 5): RVB PCR Pos | (average Ct 28.7) |
| | antibody Pos | 1/3 pools (pool of 5): RVC PCR Pos | |
| | | (Ct 34.1) | |
| | | 0/15 pigs: PSaV IFA antibody Pos | |
| G3: | 0/15 pigs: PSaV PCR Pos | 0/15 pigs: PSaV PCR Pos | 0/15 pigs: PSaV PCR Pos |
| IL25663/IL25663 | 0/15 pigs: PEDV/PDCoV/ | 0/3 pools (pool of 5): PEDV/PDCoV/ | 15/15 pigs: RVA PCR Pos |
| (N = 15) | TGEV PCR Pos | TGEV PCR Pos | (average Ct 16.4) |
| | 0/15 pigs: RVA/RVB/RVC | 3/3 pools (pool of 5): RVA PCR | 0/15 pigs: RVB PCR Pos |
| | PCR Pos | Pos (average Ct 27.8) | 15/15 pigs: RVC PCR Pos |
| | 0/15 pigs: PSaV IFA | 0/3 pools (pool of 5): RVB PCR Pos | (average Ct 19.4) |
| | antibody Pos | 1/3 pools (pool of 5): RVC PCR Pos | |
| | | (Ct 23.7) | |
| | | 0/15 pigs: PSaV IFA antibody Pos | |
| G4: | 0/15 pigs: PSaV PCR Pos | 1/15 pigs: PSaV PCR Pos (Ct 31.9) | 15/15 pigs: PSaV PCR Pos |
| Cowden/IL25663 | 0/15 pigs: PEDV/PDCoV/ | 0/3 pools (pool of 5): PEDV/PDCoV/ | (average Ct 28.8) |
| (N = 15) | TGEV PCR Pos | TGEV PCR Pos | 15/15 pigs: RVA PCR Pos |
| | 0/15 pigs: RVA/RVB/RVC | 2/3 pools (pool of 5): RVA PCR | (average Ct 12.8) |
| | PCR Pos | Pos (average Ct 31.7) | 0/15 pigs: RVB PCR Pos |
| | 0/15 pigs: PSaV IFA | 0/3 pools (pool of 5): RVB PCR Pos | 0/15 pigs: RVC PCR Pos |
| | antibody Pos | 0/3 pools (pool of 5): RVC PCR Pos | |
| | | 0/15 pigs: PSaV IFA antibody Pos | |

PCR testing was conducted on rectal swabs and porcine sapovirus IFA antibody assay was performed on serum samples.
PSaV PCR: Ct < 40 positive; Ct ≥ 40 negative
PEDV/PDCoV/TGEV PCR: Ct < 40 positive; Ct ≥ 40 negative
RVA/RVB/RVC PCR: Ct < 36 positive; Ct ≥ 36 negative

TABLE 9
Number of pigs positive for porcine sapovirus, porcine rotavirus A, or
porcine rotavirus C by PCR in rectal swabs over the course of the study

| Days post | G1: Neg/IL25663 | | | G2: IA79882/IL25663 | | | G3: IL25663/IL25663 | | |
|---|---|---|---|---|---|---|---|---|---|
| inoculation | PSaV | RVA | RVC | PSaV | RVA | RVC | PSaV | RVA | RVC |
| (DPI) | PCR+ | PCR+ | PCR+ | PCR+ | PCR+ | PCR+ | PCR+ | PCR+ | PCR+ |
| 0 DPI | 0/15 | TBD | TBD | 0/15 | TBD | TBD | 0/15 | TBD | TBD |
| 1 DPI | 0/15 | TBD | TBD | 15/15 | TBD | TBD | 15/15 | TBD | TBD |
| 2 DPI | 0/15 | TBD | TBD | 15/15 | TBD | TBD | 15/15 | TBD | TBD |
| 3 DPI | 0/15 | TBD | TBD | 15/15 | TBD | TBD | 15/15 | TBD | TBD |
| 4 DPI | 0/15 | 15/15 | 0/15 | 15/15 | 15/15 | 15/15 | 15/15 | 15/15 | 15/15 |
| 7 DPI | 0/10 | 9/10 | 0/10 | 10/10 | 10/10 | 10/10 | 10/10 | 10/10 | 9/10 |
| 10 DPI | 0/10 | 7/10 | 0/10 | 10/10 | 9/10 | 10/10 | 10/10 | 5/10 | 10/10 |
| 14 DPI | 0/10 | 6/10 | 0/10 | 10/10 | 5/10 | 4/10 | 9/10 | 4/10 | 3/10 |
| 21 DPI | 0/10 | 3/10 | 0/10 | 3/10 | 5/10 | 0/10 | 2/10 | 1/10 | 0/10 |
| 28 DPI | 0/10 | 1/10 | 0/10 | 3/10 | 3/10 | 0/10 | 1/10 | 2/10 | 0/10 |
| 35 DPI | 0/10 | 0/10 | 0/10 | 0/10 | 4/10 | 0/10 | 0/10 | 3/10 | 0/10 |
| 36 DPI | 10/10 | TBD | TBD | 10/10 | TBD | TBD | 10/10 | TBD | TBD |
| 37 DPI | 10/10 | TBD | TBD | 10/10 | TBD | TBD | 8/10 | TBD | TBD |
| 38 DPI | 10/10 | TBD | TBD | 10/10 | TBD | TBD | 4/10 | TBD | TBD |
| 39 DPI | 10/10 | TBD | TBD | 10/10 | TBD | TBD | 3/10 | TBD | TBD |
| 40 DPI | 10/10 | TBD | TBD | 10/10 | TBD | TBD | 3/10 | TBD | TBD |
| 41 DPI | 10/10 | TBD | TBD | 10/10 | TBD | TBD | 2/10 | TBD | TBD |
| 42 DPI | 10/10 | 4/10 | 0/10 | 10/10 | 8/10 | 0/10 | 2/10 | 0/10 | 0/10 |

PSaV PCR: Ct <40 positive; Ct ≥40 negative
RVA/RVB/RVC PCR: Ct <36 positive; Ct ≥36 negative

TABLE 10

| | Genomic | ORF1 | ORF1 | ORF2 | ORF2 |
|---|---|---|---|---|---|
| | RNA | coding | polypeptide | coding | polypeptide |
| Sapovirus_Pig/USA/IL25663/ | SEQ ID | SEQ ID | SEQ ID | SEQ ID | SEQ ID |
| 2022_P1_GIII_WGS; | NO: 1 | NO: 5 | NO: 9 | NO: 13 | NO: 17 |
| Sapovirus_Pig/USA/IL25663/ | | | | | |
| 2022_P5_GIII_WGS | | | | | |
| Sapovirus_Pig/USA/IL25663/ | SEQ ID | SEQ ID | | | |
| 2022_P9_WGS | NO: 21 | NO: 25 | | | |
| Sapovirus_Pig/USA/IA79982-GB/ | SEQ ID | SEQ ID | SEQ ID | SEQ ID | SEQ ID |
| 2022_P1_GIII_WGS; | NO: 2 | NO: 6 | NO: 10 | NO: 14 | NO: 18 |
| Sapovirus_Pig/IA79982-GB/ | | | | | |
| 2022_P3_GIII_WGS | | | | | |
| Sapovirus_Pig/USA/IA79982-GB/ | SEQ ID | SEQ ID | SEQ ID | | |
| 2022_P9_WGS | NO: 22 | NO: 26 | NO: 29 | | |
| Sapovirus_Pig/USA/IL67435/ | SEQ ID | SEQ ID | SEQ ID | SEQ ID | SEQ ID |
| 2022_P4_GIII_WGS | NO: 3 | NO: 7 | NO: 11 | | |
| Sapovirus_Pig/USA/IL67435/ | SEQ ID | SEQ ID | SEQ ID | NO: 15 | NO: 19 |
| 2022_P9_WGS | NO: 23 | NO: 27 | NO: 30 | | |
| Sapovirus_Pig/USA/IA43277-GG/ | SEQ ID | SEQ ID | SEQ ID | SEQ ID | SEQ ID |
| 2023_P2_GIII_WGS | NO: 4 | NO: 8 | NO: 12 | NO: 16 | NO: 20 |
| Sapovirus_Pig/USA/IA43277-GG/ | SEQ ID | SEQ ID | SEQ ID | | |
| 2023_P9_WGS | NO: 24 | NO: 28 | NO: 31 | | |

### Deposits

A deposit of the cultures of Sapovirus_Pig/USA/IL25663/2022, Sapovirus_Pig/IA79982/2022, Sapovirus_Pig/USA/IL67435/2022, and Sapovirus_Pig/USA/IA43277-GG/2023 is maintained by Iowa State University, having an address at 1800 Christensen Dr, Ames, IA 50011. Access to this deposit will be available during the pendency of the application to the Commissioner of Patents and Trademarks and persons determined by the Commissioner to be entitled thereto upon request. Upon allowance of any claims in the application, the Applicant(s) will make available to the public without restriction a deposit of the cultures with the American Type Culture Collection (ATCC), 10801 University Blvd, Manassas, Virginia, 20110. The cultures deposited with the ATCC will be taken from the same deposit maintained at Iowa State University as described above. Additionally, Applicant(s) will meet all the requirements of 37 C.F.R. § 1.801-1.809, including providing an indication of the viability of the cultures when the deposit is made. This deposit of the aforementioned cultures will be maintained in the ATCC Depository, which is a public depository, for a period of 30 years, or 5 years after the most recent request, or for the enforceable life of the patent, whichever is longer, and will be replaced if it ever becomes nonviable during that period.

1. An immunogenic composition comprising an inactivated or live attenuated porcine sapovirus (SaV), wherein the SaV comprises a polynucleotide having at least 90%, 95%, 96%, 97%, 98%, 99%, or 99.5% sequence identity to SEQ ID NO: 1, 2, 3, 4, 21, 22, 23, or 24; and
a pharmaceutically acceptable carrier.

2. The immunogenic composition of claim 1, wherein the SaV is inactivated.

3. The immunogenic composition of claim 1, wherein the SaV is a live attenuated virus.

4. The immunogenic composition of claim 3, wherein the SaV is attenuated by passaging in cell culture such that when the attenuated virus is administered to a swine it fails to cause clinical signs of SaV but is capable of inducing an immune response that immunizes the swine against pathogenic forms of SaV.

5. The immunogenic composition of claim 1, wherein the pharmaceutically acceptable carrier is a diluent, adjuvant, antimicrobial agent, preservative, inactivating agent, or a combination thereof.

6. The immunogenic composition of claim 1, wherein the SaV is not wild type.

7. The immunogenic composition of claim 1, further comprising one or more non-SaV inactivated or attenuated pathogens or antigenic material thereof.

8. A method for inducing an immune response against porcine sapovirus (SaV) in a swine comprising:
administering to the swine an immunogenic composition comprising an inactivated or live attenuated porcine SaV, wherein the SaV comprises a polynucleotide having at least 90%, 95%, 96%, 97%, 98%, 99%, or 99.5% sequence identity to SEQ ID NO: 1, 2, 3, 4, 21, 22, 23, or 24.

9. The method of claim 8, wherein the SaV is inactivated.

10. The method of claim 8, wherein the SaV is a live attenuated virus.

11. The method of claim 8, wherein the SaV is attenuated by passaging in cell culture such that when the attenuated SaV is administered to a swine it fails to cause clinical signs of SaV but is capable of inducing an immune response that immunizes the swine against pathogenic forms of SaV.

12. The method of claim 8, wherein the immunogenic composition further comprises a pharmaceutically acceptable carrier.

13. The method of claim 12, wherein the pharmaceutically acceptable carrier comprises a diluent, adjuvant, antimicrobial agent, preservative, inactivating agent, or a combination thereof.

14. The method of claim 8, wherein the immunogenic composition further comprises one or more non-SaV inactivated or attenuated pathogens or antigenic material thereof.

15. The method of claim 8, wherein the swine is a sow, gilt, boar, hog, or piglet.

16.-19. (canceled)

20. A culture of cells comprising a porcine sapovirus (SaV), wherein the SaV comprises a polynucleotide having at least 90%, 95%, 96%, 97%, 98%, 99%, or 99.5% sequence identity to SEQ ID NO: 1, 2, 3, 4, 21, 22, 23, or 24.

21. The culture of cells of claim 20, wherein the cells are LLC-PK1 cells.

22. The culture of cells of claim 20, wherein the SaV is Sapovirus_Pig/USA/IL25663-2/2022, Sapovirus_Pig/IA79982-GB/2022, Sapovirus_Pig/USA/IL67435/2022, or Sapovirus_Pig/USA/IA43277-GG/2023, wherein a representative culture has been deposited under ATCC Accession No. ______.

23.-49. (canceled)

50. An immunogenic composition comprising an inactivated or live attenuated porcine sapovirus (SaV) selected from IL25663/2022 or IA79982/2022, wherein the composition induces cross-protective immunity against heterologous SaV strains.

51. The immunogenic composition of claim 50, wherein the IL25663/2022 strain induces higher levels of viral shedding than the IA79982/2022 strain.

52. (canceled)


SEQUENCE LISTING (38 sequences)

SEQ ID NO: 1 - RNA, 7340 nt, Sapporo virus
  source 1..7340: mol_type = genomic RNA
gtgatcgtgatggctaactgccgtccgttgcctattgggcatttgcccaacaggatttttagcaccccacgactcaccccagggtgggtgtgggcgtgcactaaggaagccatgttccaactagagtggcttcaagaaccggtggtcattcgacagccggatgtttacattgcgcaaggggtggtagatgatttcttccgccccaagcgtgtcctacagggtgatccgcagctcatagcacaggtgttacttggtgatgccagtggtccgctggtcggacctgtctcaatgcagcaactaacatcgttgctgcatgaggtttcacaagcattaaacgaccacaagcatcccctcgccaacagatacaccaaaacctctttacagagatatgcagacaccttgtcaaactacatacccctagtggagatattaactggaccaaaagacctcaccccgcgcgatgtgctcgagcaactcgcggctgggcgtgaatgggagtgtgtcccagacagtgccctcaaaaaggtctttcgggacatgtggcaatacatttgtgagggctgtgacagcgtgtacatcaaactgcaggatgtgaaacgaaagataccccacattgacaccactgtgttgaaacaattcttcatcacactcacagacaccatatcaatggcaacagcactagataccaagacctggttggcacacatactgggctggcttaagcccacttgtctggtgatgatcatgcagcaacacataaactctccccaagggtgggcggccacactaactgcccttgcagagctgtactacggcatcatgccgcttaccgaggtgctcggatccatcgctggttgggtcaccgacaagtttgctgatgtaaccaccaatacctggggcaagttcaaggcttggtgggacaatctttacacaccgcaagccggcaatgacttgatcatactcgggggtgtcgttgggctggtgtacttcatggtctttggagacgcacccacccagatgttcaccaagaagctaatgaaggtgtgtgggttcatcacgtccacagtggcagcgatcaaggctgctatgtggatagtggattactttaagcagcgcgaacatgaacaccaagttcgcgtcactttggcgaggtgggccgcactccaggaggtcatcaagcagaacaggtgtgctggccttagtgaggtcgccaagttgaaagagtgctgtgaagtcttgctgaatgaggtcaccgaactaatgtacaagctgggtgcctcacctttggcggggttgatacgtagcacctctgatgtcatacagaccactatcaatgatctggctcagctaatggcgtacgacacccaacgtaagccccccgccatgattgtgtttggtgggccaccaggtataggcaagacccggttggttgaagccctggccagacagctcggggaggtcagccatttcaccatgactgtcgaccactatgacacctacacaggtaatgcagtggctatatgggatgaatttgacgtggattcaaagcaggcattcatcgaggccactatcggcatcgtcaactgtgccccctaccccttaaactgtgaccggccagaggcaaagggcagggtgttcacgtcaaagtatgtgctggccaccaccaactgtcccacaccagttatgcctgaccatccgagggctatggccttctggcgccgcataaccttcattgatgtcactgcccccacaattgaacagtggctggttgacaatcccgggcgcaaggcaccggccagcctttttaaagaggatttttcccatttgcagtgttccgtgcgtgggtacactgcctatgacgaaaagggcaacacgctcagtggcaaggtggctcgggccaagtacgtgtcagtcaacaacctgcttgacctgatcaaagagaagtacaacagtgaagccactgacatcaaacacctatggttcactgtcccacagggaattcacaagcaggcacgagacattatactgggttggctgcggttccactcctaccccaacactgtggccgacaacatacccctctcagaagtcagggaccccacctgcttcgggtatgtggtcatatctgatgtggacccgcctcggcacgtgagtgaccatgtggcacatattgaagtggaatcaatattgcgcaccgacatcatggacctcctgagagagggaggtggtggcctgttcagggcgttgaaggtcaaaagcgccccgcggaattgtgtgattaacaaagtcatgatgcaagcacaccacaccaccctgcaggtgcttacaagccaggaaccaaacccaccaaacttgccccgacccaggcgcttggtttttgttgagtcacccattgacatcattagcgccctgcgacaccacgtggggttctgcaccatcccaggcatagtcaagttgattacatcgggagtgggcctgggtgtagaaaacttgggcaattttctgcagtccatcgccggcaacgtcaggtttcctttgcagtccgagtgctcactactcaggacacccggcggtgatgtgttattctacacttcagggcaagcagcagtgtgggcgacacccgcacgttttccaatcgtcactccgggagaggcatctgttggcaaagatgtgtgttctgagtcctcctggtgggacatcctcaaggccttgttcagcacgttggtggtggccttcggccccatagccacattggtgctaaccgcgcacaacttggcctacttgaacacccgtgagaacaccctgagtgaggcaaaaggtaaaaacaaacgtggtcgcggtgcccgcaaggccatcgcccttagggacgatgagtatgatgaatggcaggacatcatccgtgactggcgcaaggaaatgactgtccaacagttcttggacttgaaagagcgcgccctttccggtgcctctgaccctgactcccaaaggtataatgcttggctggaactgcgtgctaagagattaagtgccggggcttaccaacatgcagttgtggacatcatcgggaaatcaggacacaggcgcgaggtgattcgcacacaggtgatgcgggcccctcgtgaaccaaaagcagacacttacgacagtgaggggcgtggttacgtggtgcccatgactgcccaggaaaagcacacaggctgggcagtgcacattggaaatggccgactggtcacttgcacacatgtggccaacatgtgcgatcgcgtagctgaggtggagttcaagatcacggaaacagatcgcgacacttgcatcatcactgccccactcggccacctacccagtgttgcccttggggatggaccaccggccttttacaccaccaatttccaccccatccgtgtgttggatgagggcagttgggacaccacaaccacccgtgttactggttggcgtgttgttatcaataacggcaccacaactgcccctggtgactgcggccaaccatacctgaacgcacgccgacagctcgttggtgtacatgcagcaacaagtacatgtggggtcaagaaactggtgtctagggtgcagaccaagagagcatcaaaggccacattcccttggaagggcctgcctgtcaccaatatgcccgatgctggcggcttgcccactggcacgcggtaccaccgctccatcgcgtggcccaagcagttacctgaggaaacgcacgcccccgccccctatggtgtcaatgatccccggtaccccttctcccaacaccaaatgattgccaacaacctacaaccctacaccaaaaccccagtcgcgctagaccaaaccttgctgcaacgtgctgttaaacacacaaagggttacctagaccaagttattggaacacacagatccccaaacctcacctatgcggcagcagtggattcgatggcccacgacacagcatgtgggccaaatctacctggccgcaagaaggattacatgacagatcaaggggagcccattggaccactcaaagagatgttggaggaagcttgggacatggctcaccgaggtgtcccgcgcaagcacgagtacaagcttgcactcaaggatgaactccggcccatagagaagaatgatcaaggcaagaggcgcctcctttggggttgcgatgctggggtgtcactagtggccaacgccgtgtttaagcctgtggccgagcgcctggtggacaccgtgcccatgcaccctgtggctgttggcatatgcatggacagcccccaaattgaacagatgaaccaggccttaactggtagggtgctatactgtttggattacagcaagtgggattccacgcaaaacccagccgtgacaagcgccagtgtcgacattttggcctcgtacgccgaggacacaccactcacatcggccgccatagctacactatgttcaccagccatcggtcgacttgacgacattggcttgactgtcacaacgggtctcccctctggcatgccgttcaccagcgttataaattcagtcaaccacatggtgtattttgccatggctgtgcttgaggcctacgaggagttcaaggtaccctatatgggaaacatttttgacaatgagaccgtttacacctatggggatgactgtgtttatggcctaacaccggcaacagcttccatcatgccggtggtggtcaagaacctgaccagttacggattggttccaaccgctgcagataagtcccaaaccattgaacccactgacaccccagtgtttctcaaacgcaccttttcccaaacaccttttggactgcgggcattgcttgatgaaactagtctggcgcgacagtgctactgggtcaaagccaaccgcacaactgacctctttgagccagcagctgttgatgtggaaattaggaaaaaccagctggaggtcatgctcgcatacgccagccaacatccaagggtggtgtttgacaagcttgcaagcatggttgagacaaccgcaaatgttgagggttaccaagttgtgaatgttgattgggccaacgcagtggcaacgtacaacgcatggtacggtggcactgacggtggccacgctcccaccaatgaagatgaagagccagaagtgttcgtgatggaggcacctgccccaacccgttcggcggcgagcaacccagagggtactcaaaccagcaatgaatccaggccagttcagccagccgggcccatgcccgtggccactgcccaggcccttgagatggctgttgccactggacaagtcaatgacaccatccccagtgtggttagagaaactttcagcacctacaccaatgtcacctggactacacgtcagccagcaggaaccctgctcgcccggatgaccctagggccaggcctgaatccctacacactccacctgtccgccatgtgggccggctggggagggtcatttgaaatcaaggtggtaatatcggggtctggtttgtatgcaggaaaattgctgtgcgcactcataccacccggggtggaccccagtgctgtagaccagcctggagcttttccccatgcgcttgtggatgcgcgcatcactgagggtgtcaccttcaccctcggggacgtcagggcagtggactaccacgagacaggagctggcgggaccatcgcttgcttggcactctacgtgtaccaaccactcatcaacccttttgaaactgctttgtcagccgccatggtgacaatcgagacccgccccggcccagactttgggttcaccctgctcaagcctccaaaccaaaccatggaggctggacttgaccccagatcgctcctgccccgcacggcaaggacactgcggggaaacaggtttggcaggcccatcacggccgtggtcatagttggcctagcacaccaaatcaacaggcacttctcagccgagggcaccacgctcggttggtccacagccccaattggtccttgtgtgggtcgcatcaattccaggtacaccaacaatggcggccttgctgtgctctcgatgcagcccctgagcaatgggcccctctaccccaacatcatcaaccactacccagacgtggctgcatcaagggcattcaacacaaacactaccctgaacaacgacaccacgtgtgggggggggcccatggtgatcttcaatgatgtgggtgatgtggttgaaactgtgtcctaccaaatgagattcatagcctcacaagccacctcccaaacacccacacttgttgattacatcaatgcaacatcaatgggactgtgcagctttggcaactctcggggggactttggctcaggccagctcaatgtgggcgttgaactgacctacacctgtggcaacacagcgatcaatgagaaagtcaccacattcatggatcgccaatacacatttggtgcacaggggccaaataacatcatgctctgggtggagactgtgctcggcacgcacacgggcaataacactgtgtatagctcgcaacccgacactgtgtctgccgcactgcagggtcaaccttacaatataccagatgggtacatggctgtgtggaatgttaatgcaaacagtgccgatttccagataggcctaaggcgcgatggcttctttgtcaccagtggggccattggtacgcgcatggtcatctcagaggacaccaccttcacctacgctggcattttcaccctcactacccccctcattggaccaagtgggatgacaggacggtcccttcacagctcacgatgagttgggttgcaggtgcaatgcagggtgctggcctgctaggtgacttggcaggcacgattggacagatagtgctttacaacaagcagttgaacattcagaaatcttttaatcaggcccagatggagttggccaaagaacaactaaaacaaaatcaacaaatggccaaccagttttatgattttaattctaatttacctgctaatcagtacaacagtgctgtgggtgctggctttgactcggtctctgcacgtcaattggctgggtcacacgaagtgcgctactttggtggacaacaaacacctctgctccaccaaggacagatgcatcaaatgatgttctcatcaaaacacttgatgcaagcacaaaacgtgcttggcactttttcccgtgggacacctggtgttacccccagcaaagggttgcctaaaccgcaaggcatcacccccaggccacagggcgtcacagcctccgtgcccgtggctggagtcaccaccactcacagcaaagtgtgattttctttccctggttacgcgtaccgaaccttacacagtgtggcggctgtgggg

SEQ ID NO: 2 - RNA, 7349 nt, Sapporo virus
  source 1..7349: mol_type = genomic RNA
gtgatcgtgatggctaactgccgtccgttgcctattgggcacttgcccaacaggatttttagcaccccacgactcaccccagggtgggtgtgggcgtgcactaagaaagccatgttcaagctagagtggcttcaggatccggtggtcattcgacagccggatgtttacaccgcgcaaggggtggtggatgacttcttccgccccaagcgtgttttacagggtgatccacaactcatagcacaggtgttgcttggtgatgccagcggcccgctggtcggacctgtatcaatgcagcaactaacatcgttgctgcatgaggtttcacaggcactaaatgaccacaagcatcccctggccaatagatacaccaaggcctctctacaaagatatgcggacaccttgtccaactacatacccctggtggagatattaactggaccaaaggacctcaccccgcgcgacgtgctcgagcaactcgcagctgggcgtgaatgggagtgtgtcccagacagtgccctcaagaaggtctttcgggacatgtggcaatacatttgtgagggctgtgatagcgtgtacatcaaactgcaggatgtgaagcgcaaaataccccacattgacaccactgtgttgaaacagttcttcatcacactcacagacaccatatcaatggcaacagcgctagacaccaaaacctggttggcacacatactgggttggcttaagcccacctgtttggtgatgattatgcaacaacacatcaactcaccccaggggtgggcggccacactaactgccctcgcagagctctactatggtatcatgccgctcaccgaggtgctcgggtccatcgccggttgggttaccgacaagtttgctgatgtaaccaccaacacctggggcaagttcaaggcttggtgggacaatctttacacaccgcaagctggcaatgacttgatcatactaggtggtgttgtcgggctggtgtacttcatggtttttggagatgcacccacccagatgttcaccaagaagctaatgaaggtgtgtgggttcatcacatccactgtggccgcaatcaaggctgctatgtggatagttgattacttcaagcaacgcgaacatgaacatcaggttcgcgtgaccttggccaggtgggccgcactccaggaagtcatcaagcagaacaggtgtgctggccttagtgaagtcaccaaactgaaagaatgctgtgaagtcttactgaacgaggtcaccgaattgatgtacaagctgggcgcctcgcctctagcggggttgatacgcagcacctctgatgtcatacagaccaccatcaacgacttggctcagctgatggcgtacgacacccaacgcaaaccccccgccatgatcgtgtttggtgggccaccaggcataggtaagacccggctggttgaagctctggccagacagcttggggaggtcagccacttcaccatgaccgttgaccactacgatacctacacaggtaatgcagtggcaatatgggacgaatttgatgtggattcaaagcaggcattcatcgaggccaccatcggtattgttaactgtgccccctaccccttaaactgcgaccggccagaggcaaagggcagggtattcacatcaaagtatgtgttggccaccaccaactgtcccacaccagtcatgcccgatcacccgagggctatggcgttctggcgccgcataaccttcattgacgtcactgcccccacaattgaacaatggcttgttgacaaccccgggcgcaaggcaccgcctagccttttcaaagatgacttttcccacttgcagtgttccgtccgcgggtacactgcctatgatgaaaaaggcaacacgctcagtggcaaggtggctcgggccaggtacgtgtcagtcaataacctgctcgacctgatcaaagagaaatacaacagtgaagccactgacatcaaacacctgtggttcaccgttccgcagggcattcacaagcaagcacgagatatcatactggggtggctgcggttccactcctaccccaacaccgtggctgacaacatacccctttcagaagtcagggatcccacctgcttcgggtatgtagtcatttctgatgtggacccacctcggcacgtgagtgaccatgtggcacacattgaagtggagtcaatattgcgcaccgacatcatggaccttttgagagagggaggcggaggcctgttcagggcgttgaaggttaaaagtgccccccgaaattgtgtgatcaacaaagtcatgatgcaagcgcaccacaccaccctgcaggtgctcaccagccaggaaccaaacccaccaaacctgccccgacctagacgcttggtttttgttgagtcaccaattgacatcattagcgccctgcgacaccacgtggggttctgcaccattccaggcatagtcaagttgatcacatcaggagtgggcctgggagtggaaaacctgggcaatttcttgcagtccattgccggcaacgtcagattccctttgcaatccgagtgctcactgctcaggacacctggcggtgacgtgctattttacacctcagggcaagcagcagtgtgggcgacacccgcacgttttccaatcgtcactccgggagaggcatctgttggcaaagatgtgtgttctgagtcctcctggtgggacatcctcaaggccctgtttagcacgctggtggtggccttcggccccatagccacactggtgctgaccgcacacaacctggcctacctgaacacccgtgaaaacaccttgagtgaggcaaaaggtaaaaacaagcgtggccgcggtgcccgcaaaaccatcgccctcagggacgatgagtatgatgaatggcaggacatcattcgtgactggcgcaaggaaatgactgtccaacagtttttagatctgaaagaacgcgccctttccggcgcctctgatcctgattcgcagaggtacaatgcctggctggaactgcgcgcaaagagattgagtgccggggcctaccagcatgcagttgtggacatcattgggaagtcaggacataggcgtgaggtgattcgcacgcagatgatgcgggcgccccgtgaaccaaaagcggacacctatgatagtgaggggcgtggttacgtggtgcccatgactgcccaggaaaaacacacaggctgggcagtgcacattggaaatggccgactggtcacttgcacacatgtggccaacatgtgcgatcgcgtggctgagatggagttcaagatcacagaaacggatcgcgacacttgtattatcaccgccccactcggccacctacccagtgttgcccttggggacggaccgccagccttttacaccactaatttccaccccatccgtgtgttggatgagggcagttgggacaccaccaccacccgtgtcaccggttggcgtgttgtcattaacaacggcaccacaactgcccctggtgattgcggccaaccgtacttaaacacacgccggcagctcgttggcgtgcatgctgcaacaagcacatgtggggtcaagaaactggtatccagggtgcagaccaaaagggcatcaaaggccacgttcccttggaagggcctgcctgtcgccaacatgcccgatgctgggggcctgcccactggcacgcggtaccaccgctctgtcgcgtggcccaagcagttgcctgaggagacgcatgcccctgccccctatggtgtcaatgatccccggtaccccttctctcaacaccagatgattgccaacaacctacaaccctacaccaagaccccagtcgcattagaccagaccttgctacaacgtgctgtcaaacacacgaagggttacctggaccaagtcatcggaacacacagatccccaaacctcacctacgcggcagcagtggactcgatggcccacgacacagcgtgtgggccaaatttatctggccgcaagaaggactacatgacagatcagggggaacccattggaccactcaaacaaatgttagaggaggcttgggacatggctcaccggggtgttccgcgcaaacacgagtacaagctcgcgctcaaggatgaactccggcccatagaaaagaatgatcaaggcaagaggcgcctgctttggggctgtgacgctggggtgtcgctggtggccaacgccgtgtttaaacctgtagccgagcgcttggtggacaccgtgcccatgcaccctgtagctgttggcatatgtatggacagcccccaaattgagcagatgaaccaggccctaactggtagggtgctgtactgcttggactacagcaagtgggactcaacgcagaacccagctgtgacaagcgccagtgttgacatcctggcctcgtacgctgaggacacaccactcacatcagctgccatagccacactgtgttcgccagccatcggtcgacttgacgacattggtttgaccgtcacaacgggtctcccctctggcatgccgttcaccagcgtcataaattcagtcaaccacatggtgtattttgccatggctgtgcttgaggcttatgaggagttcaaggtgccctacatggggaacatttttgacaatgagactgtttacacctacggggatgactgtgtgtatggcctgacaccggctacagcttccatcatgccggtggtggtcaaaaacctgaccagttacggactggttccaactgctgcagacaaatcccaaaccattgaacccactgacaccccagttttcctcaaacgcaccttttcccagacaccctttggactgcgggcactgcttgatgaaactagtctggcgcgacagtgctactgggttaaagctaaccgcacaacagacctttttgagccagcagctgttgatgtggagattaggaaaaaccagttggaggtcatgctcgcgtacgccagccagcatccaagggcagtgtttgacaagcttgcaaacatggttgaggcaactgcaaatgctgagggttatcaagttgtgaatgttgattgggccaacgcagtggcaacgtacaacgcatggtacggtggcactgacggtggccacgctcccaccaatgaagatgaggagccagaagtgttcgtgatggaggcgcctgccccaacccgtccggttgcgagcaacccagaaggcactgagaccagtgatgagtccaggccagttcagccgcctgggcctgtgcctgtggccgcagcacaggctctggagatggccgttgccacagggcaaatcaatgacaccatccccagtgtggtcaggcaaacattcagtacttacaccaatgttacctggaccacgcgtcaacccgcaggaaccctgcttgcccgaatgtccctagggccaggattgaacccttacacgcttcatttgtccgccatgtgggccggttggggtgggtcgtttgatattaaggtggtgatatcagggtctggcatatatgcaggcaaactgctgtgtgcactcataccacctggggttgaccctagcactgtggatcaacccggggcctttccccacgcgcttgtggatgcgcgcaccactgatggtgtttcatttaacttgggggatgtcagggcagtggattaccatgaaacaggtgctggtggaaacgtggcatgcttggcactttacgtgtaccagccgctcatcaacccctttgaaaccaccatatctgctgccatggtgacagtcgagacccgtcctggcgcagactttgggtttaccctactcaagcctccaaaccagaccatggaggtgggacttgaccccaggtcgctcctgccccgcactgcaagaacactgcgggggaacaggtttggcaggcccatcagatctgtgattatagtaggtttggcacaacaaatcaacaggcatttctccgcagaaggcaccacacttggttggtccacggccccaattggcccctgtgtaggccgcatcaacaccaagtacactggtagtgcgggcaaggtggtggctcaactgttacctttgagcaatgggcccctttacccaaacatcatcaaccactacccagacgtggctgcatcaacaactttcagtggagggtctagcataacccctgacatgacgtgtgggggaggacccatggtgctcttcaacgatgtgggtgatgtggtggagaccgtctcctaccaaatgaggttcatagcctcacaggccacatctcaaagtaccacactcaccgacaagatcaatgcaacatcaatgtcagtggtcagttttgacagctcccggaatgacttcccccaatcaaatgacaatgtgggtattgagttaacctacacttgtggcaacacaccgatcaatgggaacgtcacccagttcatggaccgccaatacacctttggcgcacaggggcccaacaacatcatgctttgggtggagtctgtgcttggcacgcacactggcaacaacagagtctacagctcacaaccagataccgtgtctgctgcactgcagggccagccctacaacataccacaggggtacatggccgtgtggaatgtcaacgcggacagtgctgactttcaaatagggctgaggcgcgatggctactttatcaccaatggagccattggcacagaaatggctatttcagatgacaccaccttcaccttcaatggaatgtacaccctaaccactcccctcattggaccaagtgggacaactggaaggtccgttcacagctcacgatgagttgggttgcaggcgcaatgcagggcgctggcctgctgggtgacctcgcgggcacaattggacaaatagtgttgtacaataagcagttgaatattaccaaatcttttaatcaggctcagttagagttagctaaagatcaaatgaaacaaaatcaacagttggctaaccagtattatgaatttaattcaaatttgcctgccaatcagtacaatagtgctgtgagtgctggttttgacgcggtttctgcacgtcaattagctgggtcgcacgaagtgcgctactttggtggacaacaaacgccattgctccaccaagggcaaatgcaacaaatgatgttttcatcaaagcatctcatgcaagcgcaaaacgtgcttggcaccttttctcggggaacacctggtgtcacccccagtaaggggctgcccaaaccacagggcgtcacgtccagaccgctcagaccacagggtgtctcagccaccatacctgtggccggagccaccaccaccaacagcagagtgtgattttctttccctggttacgcgtaccgaaccttacacagtgtggcggctgtgggg

SEQ ID NO: 3 - RNA, 7340 nt, Sapporo virus
  source 1..7340: mol_type = genomic RNA
gtgatcgtgatggctaactgccgtccgttgcctattgggcacttgcccaacaggatttttagcaccccacgactcaccccagggtgggtgtgggcgtgcaccaaggaagccatgtttgagctagagtggcttcaagatccggtggtcattcgacagccggatgtttacatcgcgcaaggggtggtggatgacttcttccgccccaagcgtgtcttacagggtgatccacagctcatagcacaggtgctgcttggtgatgccagcggcccgctggtcgggcctgtttcaatgcagcaactaacatcgttgctacatgaggtttcacaggcactaaatgaccacaagcatcccctggccaacagatacaccaaagcctctttgcaaagatatgcagacaccttgtccaactacatacctctagtggagatattaactggaccaaaggacctcaccccgcgtgacgtgctcgaacaactcgcggctgggcgtgaatgggagtgtgtcccagacagcgccctcaagaaagtttttcgggacatgtggcaatacatttgtgagggctgtgacagcgtgtacatcaaattgcaggatgtgaagcgcaagataccccacattgacaccaccgtgttgaaacagttctttattacactcacagacaccatatcaatggcaacagcgctagacaccaaaacctggttggcacacatactgggttggctcaagcccacctgtttggtgatgatcatgcaacaacacatcaactctccccaggggtgggcggccacactaactgcccttgcagagctctactatggcatcatgccgctcaccgaggtgctcgggtccattgccggttgggtcactgacaagtttgctgatgtgaccaccaacacctggggcaagttcaaagcttggtgggacaatctttacacaccgcaagccggcaatgacttgatcatactaggtggtgttgtcgggctggtgtacttcatggtttttggagatgcacccacccaaatgttcaccaagaagctgatgaaggtgtgtgggttcatcacatccactgtggcagcgatcaaggctgccatgtggatagttgattattttaagcaacgcgagcatgaacatcaggttcgcgtcaccttggccaggtgggccgcactccaggaggttatcaagcagaataggtgtgctggccttagtgaaatcaccaaactgaaggagtgctgtgaagtcttgctgaatgaggtcaccgaattgatgtacaagctgggtgcctcgcctctagcggggttgattcgtagcacctctgacgtcatacaaaccaccatcaatgatttggctcagctcatggcgtacgacacccaacgcaagccccccgccatgatcgtgtttggtgggccacctggcataggtaagacccgattggttgaagctctggccagacagcttggggaggtcagccacttcaccatgaccgtcgaccactatgacacctacacaggcaatgcagtggcaatatgggacgaatttgatgtggactcaaagcaggcattcatcgaggccaccatcggtattgtcaattgtgccccctaccctttaaactgcgaccggccagaggcaaagggcagggtgttcacgtcaaagtatgtgttggccaccaccaactgtcccacaccagtcatgcctgaccacccgagggccatggcgttctggcgtcgcataaccttcattgatgtcactgcccccacaattgaacaatggttggttgacaaccccgggcgcaaggcaccggctagcctttttaaagaggacttctcccacctgcagtgctccgtacgtgggtacacagcctatgacgaaaaaggcaacacgctcagtggcaaagtggctcgggctaggtacgtatcagtcaacaacctgctcgacctgatcaaagagaagtacaacagtgaagccactgacattaaacatctgtggttcaccgttccacagagcatccacaagcaggcacgagacatcatactggggtggctgcggttccactcctaccccaataccgtggctgacaacatacccctttcggaagtcagggaccccacctgcttcgggtatgtggtcatctctgacgtggacccgccccggcatgtgagtgaccatgtggcacacattgaagtggagtcaatactgcgcaccgacatcatggaccttttgagagagggaggcggaggcctgttcagggcgttgaaggtgaaaagcgcccctcggaattgtgtgatcaacaaagtcatgatgcaagcgcaccacaccaccctgcaggtgcttaccaaccaggaaccaaacccaccaaacctgccccgacccagacgtttggtttttgttgagtcaccaattgacatcatcagcgccctgcgacaccacgttgggttctgcaccattccaggcatagtcaagttgatcacctcgggagtgggcctgggtgtggaaaacctgggcaacttcttgcaatccatcgccggcaacgtcagattccctttgcagtccgagtgctcactactcaggacacccggcggtgatgtgctattctacacctcagggcaagcagcagtgtgggcgacacccgcacgttttccaatcatcactccgggagaggcgtctgttggcaaagatgtgtgttctgagtcctcctggtgggacatcctcaaggccttgtttagcacgctggtggtggccttcggccccatagccacattggtgctgactgcacacaacttggcctacttgaacacccgtgagaacaccttgagtgaggcaaaaggtaaaaacaagcgaggtcgcggtgcccgcaaagccatcgccctcagggacgatgagtatgatgagtggcaggacatcattcgtgactggcgcaaggaaatgactgtccaacagtttttagacctgaaagaacgcgccctttccggtgcctctgaccccgattcgcagaggtataatgcctggttggaactgcgcgcaaaaaggttgagtgccggggcctaccagcatgcagttgtagacatcattgggaaatcaggacacaggcgcgaggtgattcgcacacaggtgatgcgggcgccccgtgaaccaaaggcagacacctacgatagtgaggggcgtggttacgtggtgcccatgactgcccaggaaaaacacacaggctgggcagtgcacattggaaatggccggctggtcacttgcacacatgtggccaacatgtgcgatcgcgtggctgaggtggagttcaagatcacagaaacagatcgcgacacttgcatcatcactgccccactcggccacctacccagtgttgcccttggggatggaccaccggccttttacaccaccaactttcaccccatccgcgtgttggatgagggcagttgggataccaccaccacccgtgtcaccggttggcgtgttgtcattaacaatggcaccacaacagcccctggtgattgcggccaaccgtacttgaacgcacgccggcagctcgttggtgtgcatgcagcaacaagcacatgtggggttaagaaactggtgtctagggtgcagaccaaaagggcatcaaaggccacattcccatggaagggcctgcctgtcaccaacatgcccgatgccgggggcttgcccactggcacgcggtaccaccgctccatcgcgtggcccaagcagttgcctgaggagacacatgcccctgccccctatggcgtcaatgatccccggtaccccttctcccaacaccaaatgatcgccaacaacctacagccctacaccaaaaccccagtcgcactagaccagaccttgctgcaacgtgctgttaaacatacaaaggggtatctagaccaagtcattggaacacacagatccccaaacctcacctacgcggcagcagtggactcgatggcccatgacacagcatgtgggccaaatttgcctggccgcaagaaggattacatgacagatcagggggaacccattggaccacttaaacaaatgttggaggaagcttgggacatggctcaccgaggtgtcccgcgcaagcacgagtacaagcttgcgctcaaggatgaactccggcccatagaaaagaatgatcaaggcaagaggcgcctcctttggggctgtgacgctggggtgtcgctggtggccaacgccgtgtttaaacctgtggccgagcgcctggtggataccgtacccatgcacccggtggctgttggcatatgcatggacagcccccagattgaacagatgaaccaggccttaactggtagggtgctgtactgcctggactacagcaagtgggactccacgcaaaatccagctgtgacaagcgccagtgttgacatcttggcctcatacgctgaggacacaccactcacatcagctgccatagctacactgtgttcgccagccatcggtcgacttgacgacattggcttgactgttacaacgggtctcccctctggcatgccattcaccagtgtcataaattcagtcaaccacatggtgtattttgccatggctgtgcttgaggcttacgaggagttcaaggtgccctacatggggaacatttttgacaatgagactgtttacacttacggggatgactgtgtgtatggcctcacaccggcaacagcttccatcatgccggtggtggtcaaaaacctgaccagttacgggctggttccaactgctgcagacaaatcccaaaccattgaacccactgacaccccagttttcctcaaacgcaccttttcccaaacaccctttggactgcgggcactgcttgatgaaactagtctggcgcgacagtgctactgggttaaagctaaccgcacaacagacctttttgagccagcagctgttgacgtggaaatcaggaaaaaccagttggaggtcatgctcgcgtacgccagtcagcatccaaggatagtgtttgacaagcttgcaagtatggttgaggcaactgcaaatgccgagggctaccaagttgtgaatgttgattgggccaacgcagtggcaacgtacaacgcatggtacggtggcactgacggtggccacgctcccaccaatgaagatgaagagccagaagtgttcgtgatggaggcgcctgccccaacccgttcggtggcgagcaacccagagggtacccaaaccagcaatgaatccaggccagttcagccagccgggcccatgcccgtggccactgcccaggcccttgagatggctgttgccactggacaagtcaatgacacaatccccagtgtggtcagagaaactttcagcacctacaccaatgtcacctggactacacgtcagccagcgggaaccttgctcgcccggatgaccctagggccaggcctgaacccctacacactccacctgtctgccatgtgggctggctggggagggtcatttgaaattaaggtggtgatatcggggtctggcttgtatgcaggcaaattgctgtgcgcgctcataccacctggggtggaccccagtgctgtggaccagcccggggcttttccccatgcacttgtggatgcgcgcatcactgagggcgtcaccttcacccttggggatgtcagagcagtggattaccacgaaacaggagctggtgggaccatcgcttgcttggcactttatgtgtaccaaccactcatcaacccctttgaaactgctttgtcagccgccatggtgaccatcgagacccgccctggcccagactttgggttcaccctgcttaagcctcctaaccaaaccatggaggcaggacttgaccccagatcgcttctgccccgcacagcaaggacactgcggggaaacaggtttggcaggcccatcacaggcgtggtcatagtcggcatggcacaccagatcaacaggcacttctcagccgcgggcaccacgcttggctggtccacagccccaattggtccttgtgtaggacgtattaattctaggtacaccaacaacggcggccttgccgtgctctcgttgcaacccctaagcaatgggcccctctaccctaacatcatcaaccactacccagatgtggctgcttcaaaagctttcaacacaagcactaccctgagtgatagcaccacgtgtggtgggggacctatggtgattttcaatgatgtgggtgatgtggttgagaccgtgtcctaccaaatgagattcatagcctcacaagccacctcccaaacacccacactcgttgattacatcaatgcaacatcaatgggattggtcagttttggcaattctcggggggactttggctcaggccagctcaatgtgggcgtcgagttgacctacacctgtggcaacacagcgatcaatgagaaagtcactacgttcatggatcgccaatacacatttggtgcacagggtcccaacaacatcatgctctgggtggagactgtactcggcacgcacacgggcaacaacactgtgtacagctcgcaacccgacactgtgtctgctgcactgcagggtcagccatacaacataccagatggatacatggctgtgtggaatgttaatgcaaacagtgccgatttccagataggcttgaggcgtgatggcttctttgtcaccagtggggccattggcacgcgcatgaccatctcagaggacaccaccttcacctacgccggcatgttcaccctcaccacccccctcattggaccaagtgggatgacaggacggtcccttcacagctcaccatgagttgggttgcaggtgcaatgcaaggtgctggcctgctaggtgatctagcaggcacaattggacaagtagtgctctacaacaagcagttgaatatccagaagtcttttaatcaagcccagatggagttggccaaggaacaattgaaacaaaaccaacagatggccaaccagttttatgattttaattcaaatttacctgctaatcagtacaatagtgctgtgggtgctggctttgactcggtctctgcacgtcaactggctgggtcacatgaagtgcgttattttggtggacagcaaacacctttactccaccaagggcaaatgcaccaaatgatgttttcatcaaaacacctgatgcaagcacaaaatgtgcttggcactttttcccgtggaacgcctggtgtcacccctagcaaaggactgcctaaaccacagggcatcaaccccaggccacagggcgtcacagcctccgtgccggtggctggagccaccaccactcacagcaaagtgtgattttctttccctggttacgcgtaccgagccttacacagtgtggcggctgtgggg

SEQ ID NO: 4 - RNA, 7340 nt, Sapporo virus
  source 1..7340: mol_type = genomic RNA
gtgatcgtgatggctaactgccgtccgttgcctattgggcacttgcccaacaggatttttggcaccccacgactcaccccagggtgggtgtgggcgtgcactaaggaagccatgtttaagctagattggcttcaagatccggtggtcatccgacagccggatgtttacatcgcgcaaggggtggtggatgatttcttccgccccaagcgtgttttacagggtgatccacagctcatagcacaggtgttacttggtgatgccagcggcccactggtcgggcctgtctcaatgcagcaactaacatcgttgctgcatgaggtctcacaggcactgaatgaccacaagcaccccctggccaacagatacaccaaagcctctttgcaaagatatgcagacaccttgtccaactacatacctctagtggagatattaactggaccaaaggacctcaccccgcgtgacgtgctcgagcaactcgcggctgggcgtgaatgggagtgtgtcccagacagtgccctcaagaaggtctttcgggacatgtggcaatacatttgtgagggctgtgacagcgtgtacatcaaactacaggaagtgaagcgcaagataccccacattgacaccaccgtattgaaacagttctttatcacacttacagacaccatatcaatggcaacagcgctggacaccaagacctggttggcacacatactgggttggctcaagcccacctgtttggtgatgattatgcaacaacacatcaactccccccaggggtgggcagccacactaactgcccttgcagagctttactacggcatcatgccgctcaccgaggtgctcgggtccatcgctggttgggtcactgacaagtttgctgacgtgaccaccaacacctggggcaagttcaaggcttggtgggacaatctttacacaccgcaggctggcaatgacttgatcatactaggtggtgttgtcgggctggtgtacttcatggtttttggagatgcacccacccagatgtttaccaagaagctaatgaaggtgtgtgggttcatcacatctactgtggcagcgatcaaggctgccatgtggatagttgattactttaagcaacgcgagcacgaacaccaggttcgcgttaccttggccaggtgggccgcactccaggaggtcatcaagcagaacaggtgtgctggccttagtgaaatcgccaaactgaaggagtgctgtgaagtcttgctgaatgaggtcaccgaattgatgtataagctgggcgcctcgcctctagcggggctgattcgtagcacctctgatgtcatacagaccaccatcaatgatttagcccagctgatggcgtacgacacccaacgcaagccccccgccatgatcgtgtttggtgggccacctggcataggcaaaacccggttggttgaagctctggccagacagcttggggaggtcagccatttcaccatgaccgtcgaccactacgacacctacacaggcaacacagtggcaatatgggacgaatttgatgtggactcaaagcaggcatttatcgaggccactatcggtattgtcaattgtgccccctaccccttaaactgcgaccggccagaggcaaagggcagggtgttcacgtcaaagtatgtgttggccaccaccaactgtcccacaccagtcatgcctgaccacccgagggccatggcgttctggcgtcgcataaccttcatcgatgtcactgcccccacaattgaacaatggctgattgacaaccccgggcgcaaggcaccagccagccttttcaaggaggacttctcccacctgcagtgctccgtgcgtggttacacagcctatgacgaaaaaggcaacacgctcagtggcaaggtggctcgagctaggtatgtgtcagtcaataacctgctcgacctcatcaaagagaagtacaacagtgaagccactgacatcaaacacctgtggttcaccgtcccacaaagcatccacaagcaggcacgagacatcatactggggtggctgcggttccactcctaccccaacaccgtggctgacaacatacccctttcagaagtcagggaccccacctgcttcgggtatgtggtcatttctgacgtggacccgcctcggcatgtgagtgaccatgtggcacacattgaagtggagtcaatattgcgcaccgacatcatggaccttttgagagaggggggcggaggtctgttcagagcattgaaggtcaaaagcgcccctcggaactgtgtgattaataaagtcatgatgcaagcgcaccacaccaccctgcaagtgcttactagccaggaaccaaacccaccaaacctgccccgacccagacgtttagtttttgttgagtcaccaattgacatcattagcgccctgcgacatcacgtggggttctgcaccattccaggcatagtcaagttgattacatcgggagtgggcctgggcgtggaaaacctgggcaatttcttgcaatccatcgccggcaatgtcagatttcctctgcagtccgagtgctcactactcaggacacccggcggtgatgtgctattttacacttcagggcaagcggcagtgtgggcgacacccgcacgttttccaatcatcactccgggagaggcgtctgttggcaaagatgtgtgttctgagtcctcctggtgggacatcctcaaggccttgttcagcacgctggtagtggccttcggccccatagcaacattggtgctaactgcacacaacttggcctacttgaacacccgtgagaacaccttgagtgaggcaaaaggtaaaaacaagcgtggtcgcggtgcccgcaaagccatcgccctcagggacgatgagtatgatgaatggcaggatatcattcgtgactggcgcaaggaaatgactgtccaacagttcttggacctgaaagagcgtgccctttccggtgcctctgaccctgactcgcagaggtacaatgcctggctggaactccgcgcaaagaggttgagtgccggggcctaccagcatgcagttgtggacatcattgggaaatcaggacataggcgcgaggtgattcgcacacaggtgatgcgggcgccccgtgagccaaaagcagacacctatgacagtgaggggcgtggttacgtggtgcccatgactgcccaggaaaaacacacaggctgggcagtgcatattggaaatggccgactagtcacttgcacacatgtggccaacatgtgcgatcacgtagctgaggtggagttcaagatcacagaaacagatcgcgatacttgcatcatcactgccccgctcggccacctacccagcgttgcccttggggatggaccaccggccttttacaccaccaacttccaccccatccgtgtgttggacgagggtagctgggacaccaccaccacccgtgttaccggttggcgtgttgtcatcaacaacggcaccactactgcccctggtgattgcggtcaaccgtacttgaacgcacgccggcagctcgttggtgtgcatgcagcaacaagtacatgtggggtcaagaaactggtgtctagggtgcagaccaaaagggcatcaaaggccacgtttccttggaagggcctgcctgtcaccaacatgcccgatgccgggggcttgcccactggcacgcggtaccaccgctctatcgcgtggcccaagcagttgcctgaggagacacatgcccctgccccctatggtgtcaatgatccccggtattccttctcccaacatcaaatgatcgccaacaacctgcagccctacaccaaaaccccagtcgcattagatcaaaccttgctgcaacgtgctgtcaaacacacaaagggctacctagatcaggtcattggaacacacagatccccaaacctcacctacgcagcagcagtggactcgatggcccacgacacagcatgtgggccaaacttgcctggccgcaagaaggattacatgacagatcagggggagcccattggaccacttaaacaaatgttggaggaagcttgggacatggcccaccgaggtgtcccgcgcaaacatgagtacaagcttgcactcaaggatgaactccggcccatagaaaagaatgaccaaggcaagaggcgcctcctttggggctgcgacgctggggtgtcgctggtggccaacgccgtgttcaaacctgtggctgagcgcttggtggacactgtgcccatgcacccagtggctgttggcatatgtatggatagcccccaaattgagcagatgaaccaggccttgactggtagggtgctgtactgcctggactacagcaagtgggactccacgcaaaatccagccgtgacaagcgccagtgttgacatcttagcctcgtacgctgaggacacaccactcacgtcagctgccatagccacactgtgttcgccagccatcggtcgacttgacgacattggtttgactgttacaacgggtctcccctctggcatgccgttcaccagtgtcataaattcaatcaaccacatggtgtattttgccatggctgtgcttgaggcttacgaggagttcaaggtgccctacatggggaacatttttgataatgagactgtctacacctatggggatgactgtgtatatggcctgacaccggcaacagcctccatcatgccggtggtggtcaaaaatctgaccagttacggactggttccaactgctgcagacaaatcccaagctattgaacccactgacaccccaattttcctcaaacgcaccttttcccaaacaccctttgggttgcgggcactgcttgatgaaactagtctggcgcgacagtgctactgggtcaaggctaaccgcacaacagacctttttgagccagcagctgttgatgtggaaattaggaaaaaccaattggaggtcatgctcgcgtacgccagtcagcatccaagggcagtgtttgacaagcttgcaggcatggttgaggcaactgcaaatgccgagggttaccaagttgtgaatgttgattgggccaacgcagtggcaacgtacaacgcatggtacggtggcactaacggtggccacgctcccaccaatgaagatgaagagccagaagtgttcgtgatggaggcgcctgccccaacccgttcggtggcgagcaacccagagggtactcaaaccagcaatgaatccaggccagtccagccagccgggcccatgcccgtggccactgcccaggcccttgagatggcagttgccactggacaagtcaatgacacaatccccagcgtggtcagagaaactttcagcacctataccaatgttacctggaccacgcgccagccagcagggaccctgcttgctcggatgaccctagggccaggcctgaatccctacacactccacctgtccgccatgtgggctggctggggagggtcatttgaaatcaaggtggtgatatcggggtctggcttgtatgcaggcaagttgctgtgcgcactcataccacctggcgtagaccctagtgctgtggatcagcccggggcttttccccacgcacttgtggatgcgcgcatcactgagggtgtcaccttcacccttggggacgttagggcagtggattaccacgaaacaggagctggtgggaccatcgcttgcctggcactttatgtgtaccaaccactcatcaacccctttgaaactgctttgtcagccgccatggtaacaatcgaaacccgccctggtccagactttgggttcaccctgcttaagccccctaaccaaaccatggaggcaggacttgaccctagatcactcctgccccgcacagcaaggacactgcggggaaacaggtttggcaggcccattacaggcgtggttatagtcggcatggcataccaaatcaacaggcatttctcagctgcgggcaccacgcttggctggtccacggccccaattggtccttgtgtgggacgcatcaattctaggtacacaaacaacggcggccttgccgtgctctcgatgcaacccctgagcaatgggcccctttaccccaacatcatcaaccattacccggatgtggctgcttcaaaagcattcaacacaagcactaccctgagtgatagcaccacgtgtggtgggggacccatggtgcttttcaatgatgtaggtgatgtggttgagaacgtgtcctaccaaatgagattcatagcctcacaagccacctcccaaacacccacactcgttgattacatcaatgcaacatcaatgggattggtcagttttggcaattctcggggggactttggctcaggccagctcaatgtgggcgttgagttgacctacacctgtggtaacacagcgatcaatgagaaagtcactacgttcatggatcgccaatacacatttggtgcacaggggcccaataacatcatgctctgggtggagactgtgctcggcacgcacacgggtaacaacactgtgtacagctcgcaacccgacactgtgtctgccgcactgcagggccagccctacaacataccagatggttacatggctgtgtggaatgtcaatgcaaacagtgccgatttccagataggcttgaggcgtgatggcttctttgtcaccagtggggccattggcacgcgcatgaccatctcagatgacaccaccttcacctacgctggcatgttcaccctcaccacccctctcattggaccaagtgggatgacaggacggtcccttcacagctcaccatgagttgggttgcaggtgcaatgcaaggtgctggcctgctgggtgacctggcaggcacaattggacaaatagtgctttataacaagcagttgaatattcagaaatcttttaatcaagcccagatggagttggccaaagagcaattgaaacaaaatcaacagatggccaaccagttttatgattttaattcaaatttacctgccaatcagtacaacagtgctgtgggtgctggctttgactcggtctctgcacgtcaattggctggatcacatgaagtgcgttattttggtggacaacaaacacccttacttcaccaagggcaaatgcaccagatgatgttttcatcaaaacacctgatgcaagcacaaaatgtgctcggcactttttcccgtgggacgcctggtgtcaccccaagcaagggactgcctaaaccgcagggcatcacccccaggccacagggcgtcacagcctccgtgccggtggctggagctaccaccactcacagcaaagtgtgattttctttccctggttacgcgtaccgagccttacacagtgtggcggctgtgggg

SEQ ID NO: 5 - RNA, 6765 nt, Sapporo virus
  source 1..6765: mol_type = genomic RNA
atggctaactgccgtccgttgcctattgggcatttgcccaacaggatttttagcaccccacgactcaccccagggtgggtgtgggcgtgcactaaggaagccatgttccaactagagtggcttcaagaaccggtggtcattcgacagccggatgtttacattgcgcaaggggtggtagatgatttcttccgccccaagcgtgtcctacagggtgatccgcagctcatagcacaggtgttacttggtgatgccagtggtccgctggtcggacctgtctcaatgcagcaactaacatcgttgctgcatgaggtttcacaagcattaaacgaccacaagcatcccctcgccaacagatacaccaaaacctctttacagagatatgcagacaccttgtcaaactacatacccctagtggagatattaactggaccaaaagacctcaccccgcgcgatgtgctcgagcaactcgcggctgggcgtgaatgggagtgtgtcccagacagtgccctcaaaaaggtctttcgggacatgtggcaatacatttgtgagggctgtgacagcgtgtacatcaaactgcaggatgtgaaacgaaagataccccacattgacaccactgtgttgaaacaattcttcatcacactcacagacaccatatcaatggcaacagcactagataccaagacctggttggcacacatactgggctggcttaagcccacttgtctggtgatgatcatgcagcaacacataaactctccccaagggtgggcggccacactaactgcccttgcagagctgtactacggcatcatgccgcttaccgaggtgctcggatccatcgctggttgggtcaccgacaagtttgctgatgtaaccaccaatacctggggcaagttcaaggcttggtgggacaatctttacacaccgcaagccggcaatgacttgatcatactcgggggtgtcgttgggctggtgtacttcatggtctttggagacgcacccacccagatgttcaccaagaagctaatgaaggtgtgtgggttcatcacgtccacagtggcagcgatcaaggctgctatgtggatagtggattactttaagcagcgcgaacatgaacaccaagttcgcgtcactttggcgaggtgggccgcactccaggaggtcatcaagcagaacaggtgtgctggccttagtgaggtcgccaagttgaaagagtgctgtgaagtcttgctgaatgaggtcaccgaactaatgtacaagctgggtgcctcacctttggcggggttgatacgtagcacctctgatgtcatacagaccactatcaatgatctggctcagctaatggcgtacgacacccaacgtaagccccccgccatgattgtgtttggtgggccaccaggtataggcaagacccggttggttgaagccctggccagacagctcggggaggtcagccatttcaccatgactgtcgaccactatgacacctacacaggtaatgcagtggctatatgggatgaatttgacgtggattcaaagcaggcattcatcgaggccactatcggcatcgtcaactgtgccccctaccccttaaactgtgaccggccagaggcaaagggcagggtgttcacgtcaaagtatgtgctggccaccaccaactgtcccacaccagttatgcctgaccatccgagggctatggccttctggcgccgcataaccttcattgatgtcactgcccccacaattgaacagtggctggttgacaatcccgggcgcaaggcaccggccagcctttttaaagaggatttttcccatttgcagtgttccgtgcgtgggtacactgcctatgacgaaaagggcaacacgctcagtggcaaggtggctcgggccaagtacgtgtcagtcaacaacctgcttgacctgatcaaagagaagtacaacagtgaagccactgacatcaaacacctatggttcactgtcccacagggaattcacaagcaggcacgagacattatactgggttggctgcggttccactcctaccccaacactgtggccgacaacatacccctctcagaagtcagggaccccacctgcttcgggtatgtggtcatatctgatgtggacccgcctcggcacgtgagtgaccatgtggcacatattgaagtggaatcaatattgcgcaccgacatcatggacctcctgagagagggaggtggtggcctgttcagggcgttgaaggtcaaaagcgccccgcggaattgtgtgattaacaaagtcatgatgcaagcacaccacaccaccctgcaggtgcttacaagccaggaaccaaacccaccaaacttgccccgacccaggcgcttggtttttgttgagtcacccattgacatcattagcgccctgcgacaccacgtggggttctgcaccatcccaggcatagtcaagttgattacatcgggagtgggcctgggtgtagaaaacttgggcaattttctgcagtccatcgccggcaacgtcaggtttcctttgcagtccgagtgctcactactcaggacacccggcggtgatgtgttattctacacttcagggcaagcagcagtgtgggcgacacccgcacgttttccaatcgtcactccgggagaggcatctgttggcaaagatgtgtgttctgagtcctcctggtgggacatcctcaaggccttgttcagcacgttggtggtggccttcggccccatagccacattggtgctaaccgcgcacaacttggcctacttgaacacccgtgagaacaccctgagtgaggcaaaaggtaaaaacaaacgtggtcgcggtgcccgcaaggccatcgcccttagggacgatgagtatgatgaatggcaggacatcatccgtgactggcgcaaggaaatgactgtccaacagttcttggacttgaaagagcgcgccctttccggtgcctctgaccctgactcccaaaggtataatgcttggctggaactgcgtgctaagagattaagtgccggggcttaccaacatgcagttgtggacatcatcgggaaatcaggacacaggcgcgaggtgattcgcacacaggtgatgcgggcccctcgtgaaccaaaagcagacacttacgacagtgaggggcgtggttacgtggtgcccatgactgcccaggaaaagcacacaggctgggcagtgcacattggaaatggccgactggtcacttgcacacatgtggccaacatgtgcgatcgcgtagctgaggtggagttcaagatcacggaaacagatcgcgacacttgcatcatcactgccccactcggccacctacccagtgttgcccttggggatggaccaccggccttttacaccaccaatttccaccccatccgtgtgttggatgagggcagttgggacaccacaaccacccgtgttactggttggcgtgttgttatcaataacggcaccacaactgcccctggtgactgcggccaaccatacctgaacgcacgccgacagctcgttggtgtacatgcagcaacaagtacatgtggggtcaagaaactggtgtctagggtgcagaccaagagagcatcaaaggccacattcccttggaagggcctgcctgtcaccaatatgcccgatgctggcggcttgcccactggcacgcggtaccaccgctccatcgcgtggcccaagcagttacctgaggaaacgcacgcccccgccccctatggtgtcaatgatccccggtaccccttctcccaacaccaaatgattgccaacaacctacaaccctacaccaaaaccccagtcgcgctagaccaaaccttgctgcaacgtgctgttaaacacacaaagggttacctagaccaagttattggaacacacagatccccaaacctcacctatgcggcagcagtggattcgatggcccacgacacagcatgtgggccaaatctacctggccgcaagaaggattacatgacagatcaaggggagcccattggaccactcaaagagatgttggaggaagcttgggacatggctcaccgaggtgtcccgcgcaagcacgagtacaagcttgcactcaaggatgaactccggcccatagagaagaatgatcaaggcaagaggcgcctcctttggggttgcgatgctggggtgtcactagtggccaacgccgtgtttaagcctgtggccgagcgcctggtggacaccgtgcccatgcaccctgtggctgttggcatatgcatggacagcccccaaattgaacagatgaaccaggccttaactggtagggtgctatactgtttggattacagcaagtgggattccacgcaaaacccagccgtgacaagcgccagtgtcgacattttggcctcgtacgccgaggacacaccactcacatcggccgccatagctacactatgttcaccagccatcggtcgacttgacgacattggcttgactgtcacaacgggtctcccctctggcatgccgttcaccagcgttataaattcagtcaaccacatggtgtattttgccatggctgtgcttgaggcctacgaggagttcaaggtaccctatatgggaaacatttttgacaatgagaccgtttacacctatggggatgactgtgtttatggcctaacaccggcaacagcttccatcatgccggtggtggtcaagaacctgaccagttacggattggttccaaccgctgcagataagtcccaaaccattgaacccactgacaccccagtgtttctcaaacgcaccttttcccaaacaccttttggactgcgggcattgcttgatgaaactagtctggcgcgacagtgctactgggtcaaagccaaccgcacaactgacctctttgagccagcagctgttgatgtggaaattaggaaaaaccagctggaggtcatgctcgcatacgccagccaacatccaagggtggtgtttgacaagcttgcaagcatggttgagacaaccgcaaatgttgagggttaccaagttgtgaatgttgattgggccaacgcagtggcaacgtacaacgcatggtacggtggcactgacggtggccacgctcccaccaatgaagatgaagagccagaagtgttcgtgatggaggcacctgccccaacccgttcggcggcgagcaacccagagggtactcaaaccagcaatgaatccaggccagttcagccagccgggcccatgcccgtggccactgcccaggcccttgagatggctgttgccactggacaagtcaatgacaccatccccagtgtggttagagaaactttcagcacctacaccaatgtcacctggactacacgtcagccagcaggaaccctgctcgcccggatgaccctagggccaggcctgaatccctacacactccacctgtccgccatgtgggccggctggggagggtcatttgaaatcaaggtggtaatatcggggtctggtttgtatgcaggaaaattgctgtgcgcactcataccacccggggtggaccccagtgctgtagaccagcctggagcttttccccatgcgcttgtggatgcgcgcatcactgagggtgtcaccttcaccctcggggacgtcagggcagtggactaccacgagacaggagctggcgggaccatcgcttgcttggcactctacgtgtaccaaccactcatcaacccttttgaaactgctttgtcagccgccatggtgacaatcgagacccgccccggcccagactttgggttcaccctgctcaagcctccaaaccaaaccatggaggctggacttgaccccagatcgctcctgccccgcacggcaaggacactgcggggaaacaggtttggcaggcccatcacggccgtggtcatagttggcctagcacaccaaatcaacaggcacttctcagccgagggcaccacgctcggttggtccacagccccaattggtccttgtgtgggtcgcatcaattccaggtacaccaacaatggcggccttgctgtgctctcgatgcagcccctgagcaatgggcccctctaccccaacatcatcaaccactacccagacgtggctgcatcaagggcattcaacacaaacactaccctgaacaacgacaccacgtgtgggggggggcccatggtgatcttcaatgatgtgggtgatgtggttgaaactgtgtcctaccaaatgagattcatagcctcacaagccacctcccaaacacccacacttgttgattacatcaatgcaacatcaatgggactgtgcagctttggcaactctcggggggactttggctcaggccagctcaatgtgggcgttgaactgacctacacctgtggcaacacagcgatcaatgagaaagtcaccacattcatggatcgccaatacacatttggtgcacaggggccaaataacatcatgctctgggtggagactgtgctcggcacgcacacgggcaataacactgtgtatagctcgcaacccgacactgtgtctgccgcactgcagggtcaaccttacaatataccagatgggtacatggctgtgtggaatgttaatgcaaacagtgccgatttccagataggcctaaggcgcgatggcttctttgtcaccagtggggccattggtacgcgcatggtcatctcagaggacaccaccttcacctacgctggcattttcaccctcactacccccctcattggaccaagtgggatgacaggacggtcccttcacagctcacgatga

SEQ ID NO: 6 - RNA, 6765 nt, Sapporo virus
  source 1..6765: mol_type = genomic RNA
atggctaactgccgtccgttgcctattgggcacttgcccaacaggatttttagcaccccacgactcaccccagggtgggtgtgggcgtgcactaagaaagccatgttcaagctagagtggcttcaggatccggtggtcattcgacagccggatgtttacaccgcgcaaggggtggtggatgacttcttccgccccaagcgtgttttacagggtgatccacaactcatagcacaggtgttgcttggtgatgccagcggcccgctggtcggacctgtatcaatgcagcaactaacatcgttgctgcatgaggtttcacaggcactaaatgaccacaagcatcccctggccaatagatacaccaaggcctctctacaaagatatgcggacaccttgtccaactacatacccctggtggagatattaactggaccaaaggacctcaccccgcgcgacgtgctcgagcaactcgcagctgggcgtgaatgggagtgtgtcccagacagtgccctcaagaaggtctttcgggacatgtggcaatacatttgtgagggctgtgatagcgtgtacatcaaactgcaggatgtgaagcgcaaaataccccacattgacaccactgtgttgaaacagttcttcatcacactcacagacaccatatcaatggcaacagcgctagacaccaaaacctggttggcacacatactgggttggcttaagcccacctgtttggtgatgattatgcaacaacacatcaactcaccccaggggtgggcggccacactaactgccctcgcagagctctactatggtatcatgccgctcaccgaggtgctcgggtccatcgccggttgggttaccgacaagtttgctgatgtaaccaccaacacctggggcaagttcaaggcttggtgggacaatctttacacaccgcaagctggcaatgacttgatcatactaggtggtgttgtcgggctggtgtacttcatggtttttggagatgcacccacccagatgttcaccaagaagctaatgaaggtgtgtgggttcatcacatccactgtggccgcaatcaaggctgctatgtggatagttgattacttcaagcaacgcgaacatgaacatcaggttcgcgtgaccttggccaggtgggccgcactccaggaagtcatcaagcagaacaggtgtgctggccttagtgaagtcaccaaactgaaagaatgctgtgaagtcttactgaacgaggtcaccgaattgatgtacaagctgggcgcctcgcctctagcggggttgatacgcagcacctctgatgtcatacagaccaccatcaacgacttggctcagctgatggcgtacgacacccaacgcaaaccccccgccatgatcgtgtttggtgggccaccaggcataggtaagacccggctggttgaagctctggccagacagcttggggaggtcagccacttcaccatgaccgttgaccactacgatacctacacaggtaatgcagtggcaatatgggacgaatttgatgtggattcaaagcaggcattcatcgaggccaccatcggtattgttaactgtgccccctaccccttaaactgcgaccggccagaggcaaagggcagggtattcacatcaaagtatgtgttggccaccaccaactgtcccacaccagtcatgcccgatcacccgagggctatggcgttctggcgccgcataaccttcattgacgtcactgcccccacaattgaacaatggcttgttgacaaccccgggcgcaaggcaccgcctagccttttcaaagatgacttttcccacttgcagtgttccgtccgcgggtacactgcctatgatgaaaaaggcaacacgctcagtggcaaggtggctcgggccaggtacgtgtcagtcaataacctgctcgacctgatcaaagagaaatacaacagtgaagccactgacatcaaacacctgtggttcaccgttccgcagggcattcacaagcaagcacgagatatcatactggggtggctgcggttccactcctaccccaacaccgtggctgacaacatacccctttcagaagtcagggatcccacctgcttcgggtatgtagtcatttctgatgtggacccacctcggcacgtgagtgaccatgtggcacacattgaagtggagtcaatattgcgcaccgacatcatggaccttttgagagagggaggcggaggcctgttcagggcgttgaaggttaaaagtgccccccgaaattgtgtgatcaacaaagtcatgatgcaagcgcaccacaccaccctgcaggtgctcaccagccaggaaccaaacccaccaaacctgccccgacctagacgcttggtttttgttgagtcaccaattgacatcattagcgccctgcgacaccacgtggggttctgcaccattccaggcatagtcaagttgatcacatcaggagtgggcctgggagtggaaaacctgggcaatttcttgcagtccattgccggcaacgtcagattccctttgcaatccgagtgctcactgctcaggacacctggcggtgacgtgctattttacacctcagggcaagcagcagtgtgggcgacacccgcacgttttccaatcgtcactccgggagaggcatctgttggcaaagatgtgtgttctgagtcctcctggtgggacatcctcaaggccctgtttagcacgctggtggtggccttcggccccatagccacactggtgctgaccgcacacaacctggcctacctgaacacccgtgaaaacaccttgagtgaggcaaaaggtaaaaacaagcgtggccgcggtgcccgcaaaaccatcgccctcagggacgatgagtatgatgaatggcaggacatcattcgtgactggcgcaaggaaatgactgtccaacagtttttagatctgaaagaacgcgccctttccggcgcctctgatcctgattcgcagaggtacaatgcctggctggaactgcgcgcaaagagattgagtgccggggcctaccagcatgcagttgtggacatcattgggaagtcaggacataggcgtgaggtgattcgcacgcagatgatgcgggcgccccgtgaaccaaaagcggacacctatgatagtgaggggcgtggttacgtggtgcccatgactgcccaggaaaaacacacaggctgggcagtgcacattggaaatggccgactggtcacttgcacacatgtggccaacatgtgcgatcgcgtggctgagatggagttcaagatcacagaaacggatcgcgacacttgtattatcaccgccccactcggccacctacccagtgttgcccttggggacggaccgccagccttttacaccactaatttccaccccatccgtgtgttggatgagggcagttgggacaccaccaccacccgtgtcaccggttggcgtgttgtcattaacaacggcaccacaactgcccctggtgattgcggccaaccgtacttaaacacacgccggcagctcgttggcgtgcatgctgcaacaagcacatgtggggtcaagaaactggtatccagggtgcagaccaaaagggcatcaaaggccacgttcccttggaagggcctgcctgtcgccaacatgcccgatgctgggggcctgcccactggcacgcggtaccaccgctctgtcgcgtggcccaagcagttgcctgaggagacgcatgcccctgccccctatggtgtcaatgatccccggtaccccttctctcaacaccagatgattgccaacaacctacaaccctacaccaagaccccagtcgcattagaccagaccttgctacaacgtgctgtcaaacacacgaagggttacctggaccaagtcatcggaacacacagatccccaaacctcacctacgcggcagcagtggactcgatggcccacgacacagcgtgtgggccaaatttatctggccgcaagaaggactacatgacagatcagggggaacccattggaccactcaaacaaatgttagaggaggcttgggacatggctcaccggggtgttccgcgcaaacacgagtacaagctcgcgctcaaggatgaactccggcccatagaaaagaatgatcaaggcaagaggcgcctgctttggggctgtgacgctggggtgtcgctggtggccaacgccgtgtttaaacctgtagccgagcgcttggtggacaccgtgcccatgcaccctgtagctgttggcatatgtatggacagcccccaaattgagcagatgaaccaggccctaactggtagggtgctgtactgcttggactacagcaagtgggactcaacgcagaacccagctgtgacaagcgccagtgttgacatcctggcctcgtacgctgaggacacaccactcacatcagctgccatagccacactgtgttcgccagccatcggtcgacttgacgacattggtttgaccgtcacaacgggtctcccctctggcatgccgttcaccagcgtcataaattcagtcaaccacatggtgtattttgccatggctgtgcttgaggcttatgaggagttcaaggtgccctacatggggaacatttttgacaatgagactgtttacacctacggggatgactgtgtgtatggcctgacaccggctacagcttccatcatgccggtggtggtcaaaaacctgaccagttacggactggttccaactgctgcagacaaatcccaaaccattgaacccactgacaccccagttttcctcaaacgcaccttttcccagacaccctttggactgcgggcactgcttgatgaaactagtctggcgcgacagtgctactgggttaaagctaaccgcacaacagacctttttgagccagcagctgttgatgtggagattaggaaaaaccagttggaggtcatgctcgcgtacgccagccagcatccaagggcagtgtttgacaagcttgcaaacatggttgaggcaactgcaaatgctgagggttatcaagttgtgaatgttgattgggccaacgcagtggcaacgtacaacgcatggtacggtggcactgacggtggccacgctcccaccaatgaagatgaggagccagaagtgttcgtgatggaggcgcctgccccaacccgtccggttgcgagcaacccagaaggcactgagaccagtgatgagtccaggccagttcagccgcctgggcctgtgcctgtggccgcagcacaggctctggagatggccgttgccacagggcaaatcaatgacaccatccccagtgtggtcaggcaaacattcagtacttacaccaatgttacctggaccacgcgtcaacccgcaggaaccctgcttgcccgaatgtccctagggccaggattgaacccttacacgcttcatttgtccgccatgtgggccggttggggtgggtcgtttgatattaaggtggtgatatcagggtctggcatatatgcaggcaaactgctgtgtgcactcataccacctggggttgaccctagcactgtggatcaacccggggcctttccccacgcgcttgtggatgcgcgcaccactgatggtgtttcatttaacttgggggatgtcagggcagtggattaccatgaaacaggtgctggtggaaacgtggcatgcttggcactttacgtgtaccagccgctcatcaacccctttgaaaccaccatatctgctgccatggtgacagtcgagacccgtcctggcgcagactttgggtttaccctactcaagcctccaaaccagaccatggaggtgggacttgaccccaggtcgctcctgccccgcactgcaagaacactgcgggggaacaggtttggcaggcccatcagatctgtgattatagtaggtttggcacaacaaatcaacaggcatttctccgcagaaggcaccacacttggttggtccacggccccaattggcccctgtgtaggccgcatcaacaccaagtacactggtagtgcgggcaaggtggtggctcaactgttacctttgagcaatgggcccctttacccaaacatcatcaaccactacccagacgtggctgcatcaacaactttcagtggagggtctagcataacccctgacatgacgtgtgggggaggacccatggtgctcttcaacgatgtgggtgatgtggtggagaccgtctcctaccaaatgaggttcatagcctcacaggccacatctcaaagtaccacactcaccgacaagatcaatgcaacatcaatgtcagtggtcagttttgacagctcccggaatgacttcccccaatcaaatgacaatgtgggtattgagttaacctacacttgtggcaacacaccgatcaatgggaacgtcacccagttcatggaccgccaatacacctttggcgcacaggggcccaacaacatcatgctttgggtggagtctgtgcttggcacgcacactggcaacaacagagtctacagctcacaaccagataccgtgtctgctgcactgcagggccagccctacaacataccacaggggtacatggccgtgtggaatgtcaacgcggacagtgctgactttcaaatagggctgaggcgcgatggctactttatcaccaatggagccattggcacagaaatggctatttcagatgacaccaccttcaccttcaatggaatgtacaccctaaccactcccctcattggaccaagtgggacaactggaaggtccgttcacagctcacgatga

SEQ ID NO: 7 - RNA, 6765 nt, Sapporo virus
  source 1..6765: mol_type = genomic RNA
atggctaactgccgtccgttgcctattgggcacttgcccaacaggatttttagcaccccacgactcaccccagggtgggtgtgggcgtgcaccaaggaagccatgtttgagctagagtggcttcaagatccggtggtcattcgacagccggatgtttacatcgcgcaaggggtggtggatgacttcttccgccccaagcgtgtcttacagggtgatccacagctcatagcacaggtgctgcttggtgatgccagcggcccgctggtcgggcctgtttcaatgcagcaactaacatcgttgctacatgaggtttcacaggcactaaatgaccacaagcatcccctggccaacagatacaccaaagcctctttgcaaagatatgcagacaccttgtccaactacatacctctagtggagatattaactggaccaaaggacctcaccccgcgtgacgtgctcgaacaactcgcggctgggcgtgaatgggagtgtgtcccagacagcgccctcaagaaagtttttcgggacatgtggcaatacatttgtgagggctgtgacagcgtgtacatcaaattgcaggatgtgaagcgcaagataccccacattgacaccaccgtgttgaaacagttctttattacactcacagacaccatatcaatggcaacagcgctagacaccaaaacctggttggcacacatactgggttggctcaagcccacctgtttggtgatgatcatgcaacaacacatcaactctccccaggggtgggcggccacactaactgcccttgcagagctctactatggcatcatgccgctcaccgaggtgctcgggtccattgccggttgggtcactgacaagtttgctgatgtgaccaccaacacctggggcaagttcaaagcttggtgggacaatctttacacaccgcaagccggcaatgacttgatcatactaggtggtgttgtcgggctggtgtacttcatggtttttggagatgcacccacccaaatgttcaccaagaagctgatgaaggtgtgtgggttcatcacatccactgtggcagcgatcaaggctgccatgtggatagttgattattttaagcaacgcgagcatgaacatcaggttcgcgtcaccttggccaggtgggccgcactccaggaggttatcaagcagaataggtgtgctggccttagtgaaatcaccaaactgaaggagtgctgtgaagtcttgctgaatgaggtcaccgaattgatgtacaagctgggtgcctcgcctctagcggggttgattcgtagcacctctgacgtcatacaaaccaccatcaatgatttggctcagctcatggcgtacgacacccaacgcaagccccccgccatgatcgtgtttggtgggccacctggcataggtaagacccgattggttgaagctctggccagacagcttggggaggtcagccacttcaccatgaccgtcgaccactatgacacctacacaggcaatgcagtggcaatatgggacgaatttgatgtggactcaaagcaggcattcatcgaggccaccatcggtattgtcaattgtgccccctaccctttaaactgcgaccggccagaggcaaagggcagggtgttcacgtcaaagtatgtgttggccaccaccaactgtcccacaccagtcatgcctgaccacccgagggccatggcgttctggcgtcgcataaccttcattgatgtcactgcccccacaattgaacaatggttggttgacaaccccgggcgcaaggcaccggctagcctttttaaagaggacttctcccacctgcagtgctccgtacgtgggtacacagcctatgacgaaaaaggcaacacgctcagtggcaaagtggctcgggctaggtacgtatcagtcaacaacctgctcgacctgatcaaagagaagtacaacagtgaagccactgacattaaacatctgtggttcaccgttccacagagcatccacaagcaggcacgagacatcatactggggtggctgcggttccactcctaccccaataccgtggctgacaacatacccctttcggaagtcagggaccccacctgcttcgggtatgtggtcatctctgacgtggacccgccccggcatgtgagtgaccatgtggcacacattgaagtggagtcaatactgcgcaccgacatcatggaccttttgagagagggaggcggaggcctgttcagggcgttgaaggtgaaaagcgcccctcggaattgtgtgatcaacaaagtcatgatgcaagcgcaccacaccaccctgcaggtgcttaccaaccaggaaccaaacccaccaaacctgccccgacccagacgtttggtttttgttgagtcaccaattgacatcatcagcgccctgcgacaccacgttgggttctgcaccattccaggcatagtcaagttgatcacctcgggagtgggcctgggtgtggaaaacctgggcaacttcttgcaatccatcgccggcaacgtcagattccctttgcagtccgagtgctcactactcaggacacccggcggtgatgtgctattctacacctcagggcaagcagcagtgtgggcgacacccgcacgttttccaatcatcactccgggagaggcgtctgttggcaaagatgtgtgttctgagtcctcctggtgggacatcctcaaggccttgtttagcacgctggtggtggccttcggccccatagccacattggtgctgactgcacacaacttggcctacttgaacacccgtgagaacaccttgagtgaggcaaaaggtaaaaacaagcgaggtcgcggtgcccgcaaagccatcgccctcagggacgatgagtatgatgagtggcaggacatcattcgtgactggcgcaaggaaatgactgtccaacagtttttagacctgaaagaacgcgccctttccggtgcctctgaccccgattcgcagaggtataatgcctggttggaactgcgcgcaaaaaggttgagtgccggggcctaccagcatgcagttgtagacatcattgggaaatcaggacacaggcgcgaggtgattcgcacacaggtgatgcgggcgccccgtgaaccaaaggcagacacctacgatagtgaggggcgtggttacgtggtgcccatgactgcccaggaaaaacacacaggctgggcagtgcacattggaaatggccggctggtcacttgcacacatgtggccaacatgtgcgatcgcgtggctgaggtggagttcaagatcacagaaacagatcgcgacacttgcatcatcactgccccactcggccacctacccagtgttgcccttggggatggaccaccggccttttacaccaccaactttcaccccatccgcgtgttggatgagggcagttgggataccaccaccacccgtgtcaccggttggcgtgttgtcattaacaatggcaccacaacagcccctggtgattgcggccaaccgtacttgaacgcacgccggcagctcgttggtgtgcatgcagcaacaagcacatgtggggttaagaaactggtgtctagggtgcagaccaaaagggcatcaaaggccacattcccatggaagggcctgcctgtcaccaacatgcccgatgccgggggcttgcccactggcacgcggtaccaccgctccatcgcgtggcccaagcagttgcctgaggagacacatgcccctgccccctatggcgtcaatgatccccggtaccccttctcccaacaccaaatgatcgccaacaacctacagccctacaccaaaaccccagtcgcactagaccagaccttgctgcaacgtgctgttaaacatacaaaggggtatctagaccaagtcattggaacacacagatccccaaacctcacctacgcggcagcagtggactcgatggcccatgacacagcatgtgggccaaatttgcctggccgcaagaaggattacatgacagatcagggggaacccattggaccacttaaacaaatgttggaggaagcttgggacatggctcaccgaggtgtcccgcgcaagcacgagtacaagcttgcgctcaaggatgaactccggcccatagaaaagaatgatcaaggcaagaggcgcctcctttggggctgtgacgctggggtgtcgctggtggccaacgccgtgtttaaacctgtggccgagcgcctggtggataccgtacccatgcacccggtggctgttggcatatgcatggacagcccccagattgaacagatgaaccaggccttaactggtagggtgctgtactgcctggactacagcaagtgggactccacgcaaaatccagctgtgacaagcgccagtgttgacatcttggcctcatacgctgaggacacaccactcacatcagctgccatagctacactgtgttcgccagccatcggtcgacttgacgacattggcttgactgttacaacgggtctcccctctggcatgccattcaccagtgtcataaattcagtcaaccacatggtgtattttgccatggctgtgcttgaggcttacgaggagttcaaggtgccctacatggggaacatttttgacaatgagactgtttacacttacggggatgactgtgtgtatggcctcacaccggcaacagcttccatcatgccggtggtggtcaaaaacctgaccagttacgggctggttccaactgctgcagacaaatcccaaaccattgaacccactgacaccccagttttcctcaaacgcaccttttcccaaacaccctttggactgcgggcactgcttgatgaaactagtctggcgcgacagtgctactgggttaaagctaaccgcacaacagacctttttgagccagcagctgttgacgtggaaatcaggaaaaaccagttggaggtcatgctcgcgtacgccagtcagcatccaaggatagtgtttgacaagcttgcaagtatggttgaggcaactgcaaatgccgagggctaccaagttgtgaatgttgattgggccaacgcagtggcaacgtacaacgcatggtacggtggcactgacggtggccacgctcccaccaatgaagatgaagagccagaagtgttcgtgatggaggcgcctgccccaacccgttcggtggcgagcaacccagagggtacccaaaccagcaatgaatccaggccagttcagccagccgggcccatgcccgtggccactgcccaggcccttgagatggctgttgccactggacaagtcaatgacacaatccccagtgtggtcagagaaactttcagcacctacaccaatgtcacctggactacacgtcagccagcgggaaccttgctcgcccggatgaccctagggccaggcctgaacccctacacactccacctgtctgccatgtgggctggctggggagggtcatttgaaattaaggtggtgatatcggggtctggcttgtatgcaggcaaattgctgtgcgcgctcataccacctggggtggaccccagtgctgtggaccagcccggggcttttccccatgcacttgtggatgcgcgcatcactgagggcgtcaccttcacccttggggatgtcagagcagtggattaccacgaaacaggagctggtgggaccatcgcttgcttggcactttatgtgtaccaaccactcatcaacccctttgaaactgctttgtcagccgccatggtgaccatcgagacccgccctggcccagactttgggttcaccctgcttaagcctcctaaccaaaccatggaggcaggacttgaccccagatcgcttctgccccgcacagcaaggacactgcggggaaacaggtttggcaggcccatcacaggcgtggtcatagtcggcatggcacaccagatcaacaggcacttctcagccgcgggcaccacgcttggctggtccacagccccaattggtccttgtgtaggacgtattaattctaggtacaccaacaacggcggccttgccgtgctctcgttgcaacccctaagcaatgggcccctctaccctaacatcatcaaccactacccagatgtggctgcttcaaaagctttcaacacaagcactaccctgagtgatagcaccacgtgtggtgggggacctatggtgattttcaatgatgtgggtgatgtggttgagaccgtgtcctaccaaatgagattcatagcctcacaagccacctcccaaacacccacactcgttgattacatcaatgcaacatcaatgggattggtcagttttggcaattctcggggggactttggctcaggccagctcaatgtgggcgtcgagttgacctacacctgtggcaacacagcgatcaatgagaaagtcactacgttcatggatcgccaatacacatttggtgcacagggtcccaacaacatcatgctctgggtggagactgtactcggcacgcacacgggcaacaacactgtgtacagctcgcaacccgacactgtgtctgctgcactgcagggtcagccatacaacataccagatggatacatggctgtgtggaatgttaatgcaaacagtgccgatttccagataggcttgaggcgtgatggcttctttgtcaccagtggggccattggcacgcgcatgaccatctcagaggacaccaccttcacctacgccggcatgttcaccctcaccacccccctcattggaccaagtgggatgacaggacggtcccttcacagctcaccatga

SEQ ID NO: 8 - RNA, 6765 nt, Sapporo virus
  source 1..6765: mol_type = genomic RNA
atggctaactgccgtccgttgcctattgggcacttgcccaacaggatttttggcaccccacgactcaccccagggtgggtgtgggcgtgcactaaggaagccatgtttaagctagattggcttcaagatccggtggtcatccgacagccggatgtttacatcgcgcaaggggtggtggatgatttcttccgccccaagcgtgttttacagggtgatccacagctcatagcacaggtgttacttggtgatgccagcggcccactggtcgggcctgtctcaatgcagcaactaacatcgttgctgcatgaggtctcacaggcactgaatgaccacaagcaccccctggccaacagatacaccaaagcctctttgcaaagatatgcagacaccttgtccaactacatacctctagtggagatattaactggaccaaaggacctcaccccgcgtgacgtgctcgagcaactcgcggctgggcgtgaatgggagtgtgtcccagacagtgccctcaagaaggtctttcgggacatgtggcaatacatttgtgagggctgtgacagcgtgtacatcaaactacaggaagtgaagcgcaagataccccacattgacaccaccgtattgaaacagttctttatcacacttacagacaccatatcaatggcaacagcgctggacaccaagacctggttggcacacatactgggttggctcaagcccacctgtttggtgatgattatgcaacaacacatcaactccccccaggggtgggcagccacactaactgcccttgcagagctttactacggcatcatgccgctcaccgaggtgctcgggtccatcgctggttgggtcactgacaagtttgctgacgtgaccaccaacacctggggcaagttcaaggcttggtgggacaatctttacacaccgcaggctggcaatgacttgatcatactaggtggtgttgtcgggctggtgtacttcatggtttttggagatgcacccacccagatgtttaccaagaagctaatgaaggtgtgtgggttcatcacatctactgtggcagcgatcaaggctgccatgtggatagttgattactttaagcaacgcgagcacgaacaccaggttcgcgttaccttggccaggtgggccgcactccaggaggtcatcaagcagaacaggtgtgctggccttagtgaaatcgccaaactgaaggagtgctgtgaagtcttgctgaatgaggtcaccgaattgatgtataagctgggcgcctcgcctctagcggggctgattcgtagcacctctgatgtcatacagaccaccatcaatgatttagcccagctgatggcgtacgacacccaacgcaagccccccgccatgatcgtgtttggtgggccacctggcataggcaaaacccggttggttgaagctctggccagacagcttggggaggtcagccatttcaccatgaccgtcgaccactacgacacctacacaggcaacacagtggcaatatgggacgaatttgatgtggactcaaagcaggcatttatcgaggccactatcggtattgtcaattgtgccccctaccccttaaactgcgaccggccagaggcaaagggcagggtgttcacgtcaaagtatgtgttggccaccaccaactgtcccacaccagtcatgcctgaccacccgagggccatggcgttctggcgtcgcataaccttcatcgatgtcactgcccccacaattgaacaatggctgattgacaaccccgggcgcaaggcaccagccagccttttcaaggaggacttctcccacctgcagtgctccgtgcgtggttacacagcctatgacgaaaaaggcaacacgctcagtggcaaggtggctcgagctaggtatgtgtcagtcaataacctgctcgacctcatcaaagagaagtacaacagtgaagccactgacatcaaacacctgtggttcaccgtcccacaaagcatccacaagcaggcacgagacatcatactggggtggctgcggttccactcctaccccaacaccgtggctgacaacatacccctttcagaagtcagggaccccacctgcttcgggtatgtggtcatttctgacgtggacccgcctcggcatgtgagtgaccatgtggcacacattgaagtggagtcaatattgcgcaccgacatcatggaccttttgagagaggggggcggaggtctgttcagagcattgaaggtcaaaagcgcccctcggaactgtgtgattaataaagtcatgatgcaagcgcaccacaccaccctgcaagtgcttactagccaggaaccaaacccaccaaacctgccccgacccagacgtttagtttttgttgagtcaccaattgacatcattagcgccctgcgacatcacgtggggttctgcaccattccaggcatagtcaagttgattacatcgggagtgggcctgggcgtggaaaacctgggcaatttcttgcaatccatcgccggcaatgtcagatttcctctgcagtccgagtgctcactactcaggacacccggcggtgatgtgctattttacacttcagggcaagcggcagtgtgggcgacacccgcacgttttccaatcatcactccgggagaggcgtctgttggcaaagatgtgtgttctgagtcctcctggtgggacatcctcaaggccttgttcagcacgctggtagtggccttcggccccatagcaacattggtgctaactgcacacaacttggcctacttgaacacccgtgagaacaccttgagtgaggcaaaaggtaaaaacaagcgtggtcgcggtgcccgcaaagccatcgccctcagggacgatgagtatgatgaatggcaggatatcattcgtgactggcgcaaggaaatgactgtccaacagttcttggacctgaaagagcgtgccctttccggtgcctctgaccctgactcgcagaggtacaatgcctggctggaactccgcgcaaagaggttgagtgccggggcctaccagcatgcagttgtggacatcattgggaaatcaggacataggcgcgaggtgattcgcacacaggtgatgcgggcgccccgtgagccaaaagcagacacctatgacagtgaggggcgtggttacgtggtgcccatgactgcccaggaaaaacacacaggctgggcagtgcatattggaaatggccgactagtcacttgcacacatgtggccaacatgtgcgatcacgtagctgaggtggagttcaagatcacagaaacagatcgcgatacttgcatcatcactgccccgctcggccacctacccagcgttgcccttggggatggaccaccggccttttacaccaccaacttccaccccatccgtgtgttggacgagggtagctgggacaccaccaccacccgtgttaccggttggcgtgttgtcatcaacaacggcaccactactgcccctggtgattgcggtcaaccgtacttgaacgcacgccggcagctcgttggtgtgcatgcagcaacaagtacatgtggggtcaagaaactggtgtctagggtgcagaccaaaagggcatcaaaggccacgtttccttggaagggcctgcctgtcaccaacatgcccgatgccgggggcttgcccactggcacgcggtaccaccgctctatcgcgtggcccaagcagttgcctgaggagacacatgcccctgccccctatggtgtcaatgatccccggtattccttctcccaacatcaaatgatcgccaacaacctgcagccctacaccaaaaccccagtcgcattagatcaaaccttgctgcaacgtgctgtcaaacacacaaagggctacctagatcaggtcattggaacacacagatccccaaacctcacctacgcagcagcagtggactcgatggcccacgacacagcatgtgggccaaacttgcctggccgcaagaaggattacatgacagatcagggggagcccattggaccacttaaacaaatgttggaggaagcttgggacatggcccaccgaggtgtcccgcgcaaacatgagtacaagcttgcactcaaggatgaactccggcccatagaaaagaatgaccaaggcaagaggcgcctcctttggggctgcgacgctggggtgtcgctggtggccaacgccgtgttcaaacctgtggctgagcgcttggtggacactgtgcccatgcacccagtggctgttggcatatgtatggatagcccccaaattgagcagatgaaccaggccttgactggtagggtgctgtactgcctggactacagcaagtgggactccacgcaaaatccagccgtgacaagcgccagtgttgacatcttagcctcgtacgctgaggacacaccactcacgtcagctgccatagccacactgtgttcgccagccatcggtcgacttgacgacattggtttgactgttacaacgggtctcccctctggcatgccgttcaccagtgtcataaattcaatcaaccacatggtgtattttgccatggctgtgcttgaggcttacgaggagttcaaggtgccctacatggggaacatttttgataatgagactgtctacacctatggggatgactgtgtatatggcctgacaccggcaacagcctccatcatgccggtggtggtcaaaaatctgaccagttacggactggttccaactgctgcagacaaatcccaagctattgaacccactgacaccccaattttcctcaaacgcaccttttcccaaacaccctttgggttgcgggcactgcttgatgaaactagtctggcgcgacagtgctactgggtcaaggctaaccgcacaacagacctttttgagccagcagctgttgatgtggaaattaggaaaaaccaattggaggtcatgctcgcgtacgccagtcagcatccaagggcagtgtttgacaagcttgcaggcatggttgaggcaactgcaaatgccgagggttaccaagttgtgaatgttgattgggccaacgcagtggcaacgtacaacgcatggtacggtggcactaacggtggccacgctcccaccaatgaagatgaagagccagaagtgttcgtgatggaggcgcctgccccaacccgttcggtggcgagcaacccagagggtactcaaaccagcaatgaatccaggccagtccagccagccgggcccatgcccgtggccactgcccaggcccttgagatggcagttgccactggacaagtcaatgacacaatccccagcgtggtcagagaaactttcagcacctataccaatgttacctggaccacgcgccagccagcagggaccctgcttgctcggatgaccctagggccaggcctgaatccctacacactccacctgtccgccatgtgggctggctggggagggtcatttgaaatcaaggtggtgatatcggggtctggcttgtatgcaggcaagttgctgtgcgcactcataccacctggcgtagaccctagtgctgtggatcagcccggggcttttccccacgcacttgtggatgcgcgcatcactgagggtgtcaccttcacccttggggacgttagggcagtggattaccacgaaacaggagctggtgggaccatcgcttgcctggcactttatgtgtaccaaccactcatcaacccctttgaaactgctttgtcagccgccatggtaacaatcgaaacccgccctggtccagactttgggttcaccctgcttaagccccctaaccaaaccatggaggcaggacttgaccctagatcactcctgccccgcacagcaaggacactgcggggaaacaggtttggcaggcccattacaggcgtggttatagtcggcatggcataccaaatcaacaggcatttctcagctgcgggcaccacgcttggctggtccacggccccaattggtccttgtgtgggacgcatcaattctaggtacacaaacaacggcggccttgccgtgctctcgatgcaacccctgagcaatgggcccctttaccccaacatcatcaaccattacccggatgtggctgcttcaaaagcattcaacacaagcactaccctgagtgatagcaccacgtgtggtgggggacccatggtgcttttcaatgatgtaggtgatgtggttgagaacgtgtcctaccaaatgagattcatagcctcacaagccacctcccaaacacccacactcgttgattacatcaatgcaacatcaatgggattggtcagttttggcaattctcggggggactttggctcaggccagctcaatgtgggcgttgagttgacctacacctgtggtaacacagcgatcaatgagaaagtcactacgttcatggatcgccaatacacatttggtgcacaggggcccaataacatcatgctctgggtggagactgtgctcggcacgcacacgggtaacaacactgtgtacagctcgcaacccgacactgtgtctgccgcactgcagggccagccctacaacataccagatggttacatggctgtgtggaatgtcaatgcaaacagtgccgatttccagataggcttgaggcgtgatggcttctttgtcaccagtggggccattggcacgcgcatgaccatctcagatgacaccaccttcacctacgctggcatgttcaccctcaccacccctctcattggaccaagtgggatgacaggacggtcccttcacagctcaccatga

SEQ ID NO: 9 - protein, 2254 aa, Sapporo virus
MANCRPLPIGHLPNRIFSTPRLTPGWVWACTKEAMFQLEWLQEPVVIRQPDVYIAQGVVDDFFRPKRVLQGDPQLIAQVLLGDASGPLVGPVSMQQLTSLLHEVSQALNDHKHPLANRYTKTSLQRYADTLSNYIPLVEILTGPKDLTPRDVLEQLAAGREWECVPDSALKKVFRDMWQYICEGCDSVYIKLQDVKRKIPHIDTTVLKQFFITLTDTISMATALDTKTWLAHILGWLKPTCLVMIMQQHINSPQGWAATLTALAELYYGIMPLTEVLGSIAGWVTDKFADVTTNTWGKFKAWWDNLYTPQAGNDLIILGGVVGLVYFMVFGDAPTQMFTKKLMKVCGFITSTVAAIKAAMWIVDYFKQREHEHQVRVTLARWAALQEVIKQNRCAGLSEVAKLKECCEVLLNEVTELMYKLGASPLAGLIRSTSDVIQTTINDLAQLMAYDTQRKPPAMIVFGGPPGIGKTRLVEALARQLGEVSHFTMTVDHYDTYTGNAVAIWDEFDVDSKQAFIEATIGIVNCAPYPLNCDRPEAKGRVFTSKYVLATTNCPTPVMPDHPRAMAFWRRITFIDVTAPTIEQWLVDNPGRKAPASLFKEDFSHLQCSVRGYTAYDEKGNTLSGKVARAKYVSVNNLLDLIKEKYNSEATDIKHLWFTVPQGIHKQARDIILGWLRFHSYPNTVADNIPLSEVRDPTCFGYVVISDVDPPRHVSDHVAHIEVESILRTDIMDLLREGGGGLFRALKVKSAPRNCVINKVMMQAHHTTLQVLTSQEPNPPNLPRPRRLVFVESPIDIISALRHHVGFCTIPGIVKLITSGVGLGVENLGNFLQSIAGNVRFPLQSECSLLRTPGGDVLFYTSGQAAVWATPARFPIVTPGEASVGKDVCSESSWWDILKALFSTLVVAFGPIATLVLTAHNLAYLNTRENTLSEAKGKNKRGRGARKAIALRDDEYDEWQDIIRDWRKEMTVQQFLDLKERALSGASDPDSQRYNAWLELRAKRLSAGAYQHAVVDIIGKSGHRREVIRTQVMRAPREPKADTYDSEGRGYVVPMTAQEKHTGWAVHIGNGRLVTCTHVANMCDRVAEVEFKITETDRDTCIITAPLGHLPSVALGDGPPAFYTTNFHPIRVLDEGSWDTTTTRVTGWRVVINNGTTTAPGDCGQPYLNARRQLVGVHAATSTCGVKKLVSRVQTKRASKATFPWKGLPVTNMPDAGGLPTGTRYHRSIAWPKQLPEETHAPAPYGVNDPRYPFSQHQMIANNLQPYTKTPVALDQTLLQRAVKHTKGYLDQVIGTHRSPNLTYAAAVDSMAHDTACGPNLPGRKKDYMTDQGEPIGPLKEMLEEAWDMAHRGVPRKHEYKLALKDELRPIEKNDQGKRRLLWGCDAGVSLVANAVFKPVAERLVDTVPMHPVAVGICMDSPQIEQMNQALTGRVLYCLDYSKWDSTQNPAVTSASVDILASYAEDTPLTSAAIATLCSPAIGRLDDIGLTVTTGLPSGMPFTSVINSVNHMVYFAMAVLEAYEEFKVPYMGNIFDNETVYTYGDDCVYGLTPATASIMPVVVKNLTSYGLVPTAADKSQTIEPTDTPVFLKRTFSQTPFGLRALLDETSLARQCYWVKANRTTDLFEPAAVDVEIRKNQLEVMLAYASQHPRVVFDKLASMVETTANVEGYQVVNVDWANAVATYNAWYGGTDGGHAPTNEDEEPEVFVMEAPAPTRSAASNPEGTQTSNESRPVQPAGPMPVATAQALEMAVATGQVNDTIPSVVRETFSTYTNVTWTTRQPAGTLLARMTLGPGLNPYTLHLSAMWAGWGGSFEIKVVISGSGLYAGKLLCALIPPGVDPSAVDQPGAFPHALVDARITEGVTFTLGDVRAVDYHETGAGGTIACLALYVYQPLINPFETALSAAMVTIETRPGPDFGFTLLKPPNQTMEAGLDPRSLLPRTARTLRGNRFGRPITAVVIVGLAHQINRHFSAEGTTLGWSTAPIGPCVGRINSRYTNNGGLAVLSMQPLSNGPLYPNIINHYPDVAASRAFNTNTTLNNDTTCGGGPMVIFNDVGDVVETVSYQMRFIASQATSQTPTLVDYINATSMGLCSFGNSRGDFGSGQLNVGVELTYTCGNTAINEKVTTFMDRQYTFGAQGPNNIMLWVETVLGTHTGNNTVYSSQPDTVSAALQGQPYNIPDGYMAVWNVNANSADFQIGLRRDGFFVTSGAIGTRMVISEDTTFTYAGIFTLTTPLIGPSGMTGRSLHSSR

SEQ ID NO: 10 - protein, 2254 aa, Sapporo virus
MANCRPLPIGHLPNRIFSTPRLTPGWVWACTKKAMFKLEWLQDPVVIRQPDVYTAQGVVDDFFRPKRVLQGDPQLIAQVLLGDASGPLVGPVSMQQLTSLLHEVSQALNDHKHPLANRYTKASLQRYADTLSNYIPLVEILTGPKDLTPRDVLEQLAAGREWECVPDSALKKVFRDMWQYICEGCDSVYIKLQDVKRKIPHIDTTVLKQFFITLTDTISMATALDTKTWLAHILGWLKPTCLVMIMQQHINSPQGWAATLTALAELYYGIMPLTEVLGSIAGWVTDKFADVTTNTWGKFKAWWDNLYTPQAGNDLIILGGVVGLVYFMVFGDAPTQMFTKKLMKVCGFITSTVAAIKAAMWIVDYFKQREHEHQVRVTLARWAALQEVIKQNRCAGLSEVTKLKECCEVLLNEVTELMYKLGASPLAGLIRSTSDVIQTTINDLAQLMAYDTQRKPPAMIVFGGPPGIGKTRLVEALARQLGEVSHFTMTVDHYDTYTGNAVAIWDEFDVDSKQAFIEATIGIVNCAPYPLNCDRPEAKGRVFTSKYVLATTNCPTPVMPDHPRAMAFWRRITFIDVTAPTIEQWLVDNPGRKAPPSLFKDDFSHLQCSVRGYTAYDEKGNTLSGKVARARYVSVNNLLDLIKEKYNSEATDIKHLWFTVPQGIHKQARDIILGWLRFHSYPNTVADNIPLSEVRDPTCFGYVVISDVDPPRHVSDHVAHIEVESILRTDIMDLLREGGGGLFRALKVKSAPRNCVINKVMMQAHHTTLQVLTSQEPNPPNLPRPRRLVFVESPIDIISALRHHVGFCTIPGIVKLITSGVGLGVENLGNFLQSIAGNVRFPLQSECSLLRTPGGDVLFYTSGQAAVWATPARFPIVTPGEASVGKDVCSESSWWDILKALFSTLVVAFGPIATLVLTAHNLAYLNTRENTLSEAKGKNKRGRGARKTIALRDDEYDEWQDIIRDWRKEMTVQQFLDLKERALSGASDPDSQRYNAWLELRAKRLSAGAYQHAVVDIIGKSGHRREVIRTQMMRAPREPKADTYDSEGRGYVVPMTAQEKHTGWAVHIGNGRLVTCTHVANMCDRVAEMEFKITETDRDTCIITAPLGHLPSVALGDGPPAFYTTNFHPIRVLDEGSWDTTTTRVTGWRVVINNGTTTAPGDCGQPYLNTRRQLVGVHAATSTCGVKKLVSRVQTKRASKATFPWKGLPVANMPDAGGLPTGTRYHRSVAWPKQLPEETHAPAPYGVNDPRYPFSQHQMIANNLQPYTKTPVALDQTLLQRAVKHTKGYLDQVIGTHRSPNLTYAAAVDSMAHDTACGPNLSGRKKDYMTDQGEPIGPLKQMLEEAWDMAHRGVPRKHEYKLALKDELRPIEKNDQGKRRLLWGCDAGVSLVANAVFKPVAERLVDTVPMHPVAVGICMDSPQIEQMNQALTGRVLYCLDYSKWDSTQNPAVTSASVDILASYAEDTPLTSAAIATLCSPAIGRLDDIGLTVTTGLPSGMPFTSVINSVNHMVYFAMAVLEAYEEFKVPYMGNIFDNETVYTYGDDCVYGLTPATASIMPVVVKNLTSYGLVPTAADKSQTIEPTDTPVFLKRTFSQTPFGLRALLDETSLARQCYWVKANRTTDLFEPAAVDVEIRKNQLEVMLAYASQHPRAVFDKLANMVEATANAEGYQVVNVDWANAVATYNAWYGGTDGGHAPTNEDEEPEVFVMEAPAPTRPVASNPEGTETSDESRPVQPPGPVPVAAAQALEMAVATGQINDTIPSVVRQTFSTYTNVTWTTRQPAGTLLARMSLGPGLNPYTLHLSAMWAGWGGSFDIKVVISGSGIYAGKLLCALIPPGVDPSTVDQPGAFPHALVDARTTDGVSFNLGDVRAVDYHETGAGGNVACLALYVYQPLINPFETTISAAMVTVETRPGADFGFTLLKPPNQTMEVGLDPRSLLPRTARTLRGNRFGRPIRSVIIVGLAQQINRHFSAEGTTLGWSTAPIGPCVGRINTKYTGSAGKVVAQLLPLSNGPLYPNIINHYPDVAASTTFSGGSSITPDMTCGGGPMVLFNDVGDVVETVSYQMRFIASQATSQSTTLTDKINATSMSVVSFDSSRNDFPQSNDNVGIELTYTCGNTPINGNVTQFMDRQYTFGAQGPNNIMLWVESVLGTHTGNNRVYSSQPDTVSAALQGQPYNIPQGYMAVWNVNADSADFQIGLRRDGYFITNGAIGTEMAISDDTTFTFNGMYTLTTPLIGPSGTTGRSVHSSR

SEQ ID NO: 11 - protein, 2254 aa, Sapporo virus
MANCRPLPIGHLPNRIFSTPRLTPGWVWACTKEAMFELEWLQDPVVIRQPDVYIAQGVVDDFFRPKRVLQGDPQLIAQVLLGDASGPLVGPVSMQQLTSLLHEVSQALNDHKHPLANRYTKASLQRYADTLSNYIPLVEILTGPKDLTPRDVLEQLAAGREWECVPDSALKKVFRDMWQYICEGCDSVYIKLQDVKRKIPHIDTTVLKQFFITLTDTISMATALDTKTWLAHILGWLKPTCLVMIMQQHINSPQGWAATLTALAELYYGIMPLTEVLGSIAGWVTDKFADVTTNTWGKFKAWWDNLYTPQAGNDLIILGGVVGLVYFMVFGDAPTQMFTKKLMKVCGFITSTVAAIKAAMWIVDYFKQREHEHQVRVTLARWAALQEVIKQNRCAGLSEITKLKECCEVLLNEVTELMYKLGASPLAGLIRSTSDVIQTTINDLAQLMAYDTQRKPPAMIVFGGPPGIGKTRLVEALARQLGEVSHFTMTVDHYDTYTGNAVAIWDEFDVDSKQAFIEATIGIVNCAPYPLNCDRPEAKGRVFTSKYVLATTNCPTPVMPDHPRAMAFWRRITFIDVTAPTIEQWLVDNPGRKAPASLFKEDFSHLQCSVRGYTAYDEKGNTLSGKVARARYVSVNNLLDLIKEKYNSEATDIKHLWFTVPQSIHKQARDIILGWLRFHSYPNTVADNIPLSEVRDPTCFGYVVISDVDPPRHVSDHVAHIEVESILRTDIMDLLREGGGGLFRALKVKSAPRNCVINKVMMQAHHTTLQVLTNQEPNPPNLPRPRRLVFVESPIDIISALRHHVGFCTIPGIVKLITSGVGLGVENLGNFLQSIAGNVRFPLQSECSLLRTPGGDVLFYTSGQAAVWATPARFPIITPGEASVGKDVCSESSWWDILKALFSTLVVAFGPIATLVLTAHNLAYLNTRENTLSEAKGKNKRGRGARKAIALRDDEYDEWQDIIRDWRKEMTVQQFLDLKERALSGASDPDSQRYNAWLELRAKRLSAGAYQHAVVDIIGKSGHRREVIRTQVMRAPREPKADTYDSEGRGYVVPMTAQEKHTGWAVHIGNGRLVTCTHVANMCDRVAEVEFKITETDRDTCIITAPLGHLPSVALGDGPPAFYTTNFHPIRVLDEGSWDTTTTRVTGWRVVINNGTTTAPGDCGQPYLNARRQLVGVHAATSTCGVKKLVSRVQTKRASKATFPWKGLPVTNMPDAGGLPTGTRYHRSIAWPKQLPEETHAPAPYGVNDPRYPFSQHQMIANNLQPYTKTPVALDQTLLQRAVKHTKGYLDQVIGTHRSPNLTYAAAVDSMAHDTACGPNLPGRKKDYMTDQGEPIGPLKQMLEEAWDMAHRGVPRKHEYKLALKDELRPIEKNDQGKRRLLWGCDAGVSLVANAVFKPVAERLVDTVPMHPVAVGICMDSPQIEQMNQALTGRVLYCLDYSKWDSTQNPAVTSASVDILASYAEDTPLTSAAIATLCSPAIGRLDDIGLTVTTGLPSGMPFTSVINSVNHMVYFAMAVLEAYEEFKVPYMGNIFDNETVYTYGDDCVYGLTPATASIMPVVVKNLTSYGLVPTAADKSQTIEPTDTPVFLKRTFSQTPFGLRALLDETSLARQCYWVKANRTTDLFEPAAVDVEIRKNQLEVMLAYASQHPRIVFDKLASMVEATANAEGYQVVNVDWANAVATYNAWYGGTDGGHAPTNEDEEPEVFVMEAPAPTRSVASNPEGTQTSNESRPVQPAGPMPVATAQALEMAVATGQVNDTIPSVVRETFSTYTNVTWTTRQPAGTLLARMTLGPGLNPYTLHLSAMWAGWGGSFEIKVVISGSGLYAGKLLCALIPPGVDPSAVDQPGAFPHALVDARITEGVTFTLGDVRAVDYHETGAGGTIACLALYVYQPLINPFETALSAAMVTIETRPGPDFGFTLLKPPNQTMEAGLDPRSLLPRTARTLRGNRFGRPITGVVIVGMAHQINRHFSAAGTTLGWSTAPIGPCVGRINSRYTNNGGLAVLSLQPLSNGPLYPNIINHYPDVAASKAFNTSTTLSDSTTCGGGPMVIFNDVGDVVETVSYQMRFIASQATSQTPTLVDYINATSMGLVSFGNSRGDFGSGQLNVGVELTYTCGNTAINEKVTTFMDRQYTFGAQGPNNIMLWVETVLGTHTGNNTVYSSQPDTVSAALQGQPYNIPDGYMAVWNVNANSADFQIGLRRDGFFVTSGAIGTRMTISEDTTFTYAGMFTLTTPLIGPSGMTGRSLHSSP

SEQ ID NO: 12 - protein, 2254 aa, Sapporo virus
MANCRPLPIGHLPNRIFGTPRLTPGWVWACTKEAMFKLDWLQDPVVIRQPDVYIAQGVVDDFFRPKRVLQGDPQLIAQVLLGDASGPLVGPVSMQQLTSLLHEVSQALNDHKHPLANRYTKASLQRYADTLSNYIPLVEILTGPKDLTPRDVLEQLAAGREWECVPDSALKKVFRDMWQYICEGCDSVYIKLQEVKRKIPHIDTTVLKQFFITLTDTISMATALDTKTWLAHILGWLKPTCLVMIMQQHINSPQGWAATLTALAELYYGIMPLTEVLGSIAGWVTDKFADVTTNTWGKFKAWWDNLYTPQAGNDLIILGGVVGLVYFMVFGDAPTQMFTKKLMKVCGFITSTVAAIKAAMWIVDYFKQREHEHQVRVTLARWAALQEVIKQNRCAGLSEIAKLKECCEVLLNEVTELMYKLGASPLAGLIRSTSDVIQTTINDLAQLMAYDTQRKPPAMIVFGGPPGIGKTRLVEALARQLGEVSHFTMTVDHYDTYTGNTVAIWDEFDVDSKQAFIEATIGIVNCAPYPLNCDRPEAKGRVFTSKYVLATTNCPTPVMPDHPRAMAFWRRITFIDVTAPTIEQWLIDNPGRKAPASLFKEDFSHLQCSVRGYTAYDEKGNTLSGKVARARYVSVNNLLDLIKEKYNSEATDIKHLWFTVPQSIHKQARDIILGWLRFHSYPNTVADNIPLSEVRDPTCFGYVVISDVDPPRHVSDHVAHIEVESILRTDIMDLLREGGGGLFRALKVKSAPRNCVINKVMMQAHHTTLQVLTSQEPNPPNLPRPRRLVFVESPIDIISALRHHVGFCTIPGIVKLITSGVGLGVENLGNFLQSIAGNVRFPLQSECSLLRTPGGDVLFYTSGQAAVWATPARFPIITPGEASVGKDVCSESSWWDILKALFSTLVVAFGPIATLVLTAHNLAYLNTRENTLSEAKGKNKRGRGARKAIALRDDEYDEWQDIIRDWRKEMTVQQFLDLKERALSGASDPDSQRYNAWLELRAKRLSAGAYQHAVVDIIGKSGHRREVIRTQVMRAPREPKADTYDSEGRGYVVPMTAQEKHTGWAVHIGNGRLVTCTHVANMCDHVAEVEFKITETDRDTCIITAPLGHLPSVALGDGPPAFYTTNFHPIRVLDEGSWDTTTTRVTGWRVVINNGTTTAPGDCGQPYLNARRQLVGVHAATSTCGVKKLVSRVQTKRASKATFPWKGLPVTNMPDAGGLPTGTRYHRSIAWPKQLPEETHAPAPYGVNDPRYSFSQHQMIANNLQPYTKTPVALDQTLLQRAVKHTKGYLDQVIGTHRSPNLTYAAAVDSMAHDTACGPNLPGRKKDYMTDQGEPIGPLKQMLEEAWDMAHRGVPRKHEYKLALKDELRPIEKNDQGKRRLLWGCDAGVSLVANAVFKPVAERLVDTVPMHPVAVGICMDSPQIEQMNQALTGRVLYCLDYSKWDSTQNPAVTSASVDILASYAEDTPLTSAAIATLCSPAIGRLDDIGLTVTTGLPSGMPFTSVINSINHMVYFAMAVLEAYEEFKVPYMGNIFDNETVYTYGDDCVYGLTPATASIMPVVVKNLTSYGLVPTAADKSQAIEPTDTPIFLKRTFSQTPFGLRALLDETSLARQCYWVKANRTTDLFEPAAVDVEIRKNQLEVMLAYASQHPRAVFDKLAGMVEATANAEGYQVVNVDWANAVATYNAWYGGTNGGHAPTNEDEEPEVFVMEAPAPTRSVASNPEGTQTSNESRPVQPAGPMPVATAQALEMAVATGQVNDTIPSVVRETFSTYTNVTWTTRQPAGTLLARMTLGPGLNPYTLHLSAMWAGWGGSFEIKVVISGSGLYAGKLLCALIPPGVDPSAVDQPGAFPHALVDARITEGVTFTLGDVRAVDYHETGAGGTIACLALYVYQPLINPFETALSAAMVTIETRPGPDFGFTLLKPPNQTMEAGLDPRSLLPRTARTLRGNRFGRPITGVVIVGMAYQINRHFSAAGTTLGWSTAPIGPCVGRINSRYTNNGGLAVLSMQPLSNGPLYPNIINHYPDVAASKAFNTSTTLSDSTTCGGGPMVLFNDVGDVVENVSYQMRFIASQATSQTPTLVDYINATSMGLVSFGNSRGDFGSGQLNVGVELTYTCGNTAINEKVTTFMDRQYTFGAQGPNNIMLWVETVLGTHTGNNTVYSSQPDTVSAALQGQPYNIPDGYMAVWNVNANSADFQIGLRRDGFFVTSGAIGTRMTISDDTTFTYAGMFTLTTPLIGPSGMTGRSLHSSP

SEQ ID NO: 13 - RNA, 516 nt, Sapporo virus
  source 1..516: mol_type = genomic RNA
atgagttgggttgcaggtgcaatgcagggtgctggcctgctaggtgacttggcaggcacgattggacagatagtgctttacaacaagcagttgaacattcagaaatcttttaatcaggcccagatggagttggccaaagaacaactaaaacaaaatcaacaaatggccaaccagttttatgattttaattctaatttacctgctaatcagtacaacagtgctgtgggtgctggctttgactcggtctctgcacgtcaattggctgggtcacacgaagtgcgctactttggtggacaacaaacacctctgctccaccaaggacagatgcatcaaatgatgttctcatcaaaacacttgatgcaagcacaaaacgtgcttggcactttttcccgtgggacacctggtgttacccccagcaaagggttgcctaaaccgcaaggcatcacccccaggccacagggcgtcacagcctccgtgcccgtggctggagtcaccaccactcacagcaaagtgtga

SEQ ID NO: 14 - RNA, 525 nt, Sapporo virus
  source 1..525: mol_type = genomic RNA
atgagttgggttgcaggcgcaatgcagggcgctggcctgctgggtgacctcgcgggcacaattggacaaatagtgttgtacaataagcagttgaatattaccaaatcttttaatcaggctcagttagagttagctaaagatcaaatgaaacaaaatcaacagttggctaaccagtattatgaatttaattcaaatttgcctgccaatcagtacaatagtgctgtgagtgctggttttgacgcggtttctgcacgtcaattagctgggtcgcacgaagtgcgctactttggtggacaacaaacgccattgctccaccaagggcaaatgcaacaaatgatgttttcatcaaagcatctcatgcaagcgcaaaacgtgcttggcaccttttctcggggaacacctggtgtcacccccagtaaggggctgcccaaaccacagggcgtcacgtccagaccgctcagaccacagggtgtctcagccaccatacctgtggccggagccaccaccaccaacagcagagtgtga

SEQ ID NO: 15 - RNA, 516 nt, Sapporo virus
  source 1..516: mol_type = genomic RNA
atgagttgggttgcaggtgcaatgcaaggtgctggcctgctaggtgatctagcaggcacaattggacaagtagtgctctacaacaagcagttgaatatccagaagtcttttaatcaagcccagatggagttggccaaggaacaattgaaacaaaaccaacagatggccaaccagttttatgattttaattcaaatttacctgctaatcagtacaatagtgctgtgggtgctggctttgactcggtctctgcacgtcaactggctgggtcacatgaagtgcgttattttggtggacagcaaacacctttactccaccaagggcaaatgcaccaaatgatgttttcatcaaaacacctgatgcaagcacaaaatgtgcttggcactttttcccgtggaacgcctggtgtcacccctagcaaaggactgcctaaaccacagggcatcaaccccaggccacagggcgtcacagcctccgtgccggtggctggagccaccaccactcacagcaaagtgtga

SEQ ID NO: 16 - RNA, 516 nt, Sapporo virus
  source 1..516: mol_type = genomic RNA
atgagttgggttgcaggtgcaatgcaaggtgctggcctgctgggtgacctggcaggcacaattggacaaatagtgctttataacaagcagttgaatattcagaaatcttttaatcaagcccagatggagttggccaaagagcaattgaaacaaaatcaacagatggccaaccagttttatgattttaattcaaatttacctgccaatcagtacaacagtgctgtgggtgctggctttgactcggtctctgcacgtcaattggctggatcacatgaagtgcgttattttggtggacaacaaacacccttacttcaccaagggcaaatgcaccagatgatgttttcatcaaaacacctgatgcaagcacaaaatgtgctcggcactttttcccgtgggacgcctggtgtcaccccaagcaagggactgcctaaaccgcagggcatcacccccaggccacagggcgtcacagcctccgtgccggtggctggagctaccaccactcacagcaaagtgtga

SEQ ID NO: 17 - protein, 171 aa, Sapporo virus
MSWVAGAMQGAGLLGDLAGTIGQIVLYNKQLNIQKSFNQAQMELAKEQLKQNQQMANQFYDFNSNLPANQYNSAVGAGFDSVSARQLAGSHEVRYFGGQQTPLLHQGQMHQMMFSSKHLMQAQNVLGTFSRGTPGVTPSKGLPKPQGITPRPQGVTASVPVAGVTTTHSKV

SEQ ID NO: 18 - protein, 174 aa, Sapporo virus
MSWVAGAMQGAGLLGDLAGTIGQIVLYNKQLNITKSFNQAQLELAKDQMKQNQQLANQYYEFNSNLPANQYNSAVSAGFDAVSARQLAGSHEVRYFGGQQTPLLHQGQMQQMMFSSKHLMQAQNVLGTFSRGTPGVTPSKGLPKPQGVTSRPLRPQGVSATIPVAGATTTNSRV

SEQ ID NO: 19 - protein, 171 aa, Sapporo virus
MSWVAGAMQGAGLLGDLAGTIGQVVLYNKQLNIQKSFNQAQMELAKEQLKQNQQMANQFYDFNSNLPANQYNSAVGAGFDSVSARQLAGSHEVRYFGGQQTPLLHQGQMHQMMFSSKHLMQAQNVLGTFSRGTPGVTPSKGLPKPQGINPRPQGVTASVPVAGATTTHSKV

SEQ ID NO: 20 - protein, 171 aa, Sapporo virus
MSWVAGAMQGAGLLGDLAGTIGQIVLYNKQLNIQKSFNQAQMELAKEQLKQNQQMANQFYDFNSNLPANQYNSAVGAGFDSVSARQLAGSHEVRYFGGQQTPLLHQGQMHQMMFSSKHLMQAQNVLGTFSRGTPGVTPSKGLPKPQGITPRPQGVTASVPVAGATTTHSKV

SEQ ID NO: 21 - RNA, 7340 nt, Sapporo virus
  source 1..7340: mol_type = genomic RNA
gtgatcgtgatggctaattgccgtccgttgcctattgggcatttgcccaacaggatttttagcaccccacgactcaccccagggtgggtgtgggcgtgcactaaggaagccatgttccaactagagtggcttcaagaaccggtggtcattcgacagccggatgtttacattgcgcaaggggtggtagatgatttcttccgccccaagcgtgtcctacagggtgatccgcagctcatagcacaggtgttacttggtgatgccagtggtccgctggtcggacctgtctcaatgcagcaactaacatcgttgctgcatgaggtttcacaagcattaaacgaccacaagcatcccctcgccaacagatacaccaaaacctctttacagagatatgcagacaccttgtcaaactacatacccctagtggagatattaactggaccaaaagacctcaccccgcgcgatgtgctcgagcaactcgcggctgggcgtgaatgggagtgtgtcccagacagtgccctcaaaaaggtctttcgggacatgtggcaatacatttgtgagggctgtgacagcgtgtacatcaaactgcaggatgtgaaacgaaagataccccacattgacaccactgtgttgaaacaattcttcatcacactcacagacaccatatcaatggcaacagcactagataccaagacctggttggcacacatactgggctggcttaagcccacttgtctggtgatgatcatgcagcaacacataaactctccccaagggtgggcggccacactaactgcccttgcagagctgtactacggcatcatgccgcttaccgaggtgctcggatccatcgctggttgggtcaccgacaagtttgctgatgtaaccaccaatacctggggcaagttcaaggcttggtgggacaatctttacacaccgcaagccggcaatgacttgatcatactcgggggtgtcgttgggctggtgtacttcatggtctttggagacgcacccacccagatgttcaccaagaagctaatgaaggtgtgtgggttcatcacgtccacagtggcagcgatcaaggctgctatgtggatagtggattactttaagcagcgcgaacatgaacaccaagttcgcgtcactttggcgaggtgggccgcactccaggaggtcatcaagcagaacaggtgtgctggccttagtgaggtcgccaagttgaaagagtgctgtgaagtcttgctgaatgaggtcaccgaactaatgtacaagctgggtgcctcacctttggcggggttgatacgtagcacctctgatgtcatacagaccactatcaatgatctggctcagctaatggcgtacgacacccaacgtaagccccccgccatgattgtgtttggtgggccaccaggtataggcaagacccggttggttgaagccctggccagacagctcggggaggtcagccatttcaccatgactgtcgaccactatgacacctacacaggtaatgcagtggctatatgggatgaatttgacgtggattcaaagcaggcattcatcgaggccactatcggcatcgtcaactgtgccccctaccccttaaactgtgaccggccagaggcaaagggcagggtgttcacgtcaaagtatgtgctggccaccaccaactgtcccacaccagttatgcctgaccatccgagggctatggccttctggcgccgcataaccttcattgatgtcactgcccccacaattgaacagtggctggttgacaatcccgggcgcaaggcaccggccagcctttttaaagaggatttttcccatttgcagtgttccgtgcgtgggtacactgcctatgacgaaaagggcaacacgctcagtggcaaggtggctcgggccaagtacgtgtcagtcaacaacctgcttgacctgatcaaagagaagtacaacagtgaagccactgacatcaaacacctatggttcactgtcccacagggaattcacaagcaggcacgagacattatactgggttggctgcggttccactcctaccccaacactgtggccgacaacatacccctctcagaagtcagggaccccacctgcttcgggtatgtggtcatatctgatgtggacccgcctcggcacgtgagtgaccatgtggcacatattgaagtggaatcaatattgcgcaccgacatcatggacctcctgagagagggaggtggtggcctgttcagggcgttgaaggtcaaaagcgccccgcggaattgtgtgattaacaaagtcatgatgcaagcacaccacaccaccctgcaggtgcttacaagccaggaaccaaacccaccaaacttgccccgacccaggcgcttggtttttgttgagtcacccattgacatcattagcgccctgcgacaccacgtggggttctgcaccatcccaggcatagtcaagttgattacatcgggagtgggcctgggtgtagaaaacttgggcaattttctgcagtccatcgccggcaacgtcaggtttcctttgcagtccgagtgctcactactcaggacacccggcggtgatgtgttattctacacttcagggcaagcagcagtgtgggcgacacccgcacgttttccaatcgtcactccgggagaggcatctgttggcaaagatgtgtgttctgagtcctcctggtgggacatcctcaaggccttgttcagcacgttggtggtggccttcggccccatagccacattggtgctaaccgcgcacaacttggcctacttgaacacccgtgagaacaccctgagtgaggcaaaaggtaaaaacaaacgtggtcgcggtgcccgcaaggccatcgcccttagggacgatgagtatgatgaatggcaggacatcatccgtgactggcgcaaggaaatgactgtccaacagttcttggacttgaaagagcgcgccctttccggtgcctctgaccctgactcccaaaggtataatgcttggctggaactgcgtgctaagagattaagtgccggggcttaccaacatgcagttgtggacatcatcgggaaatcaggacacaggcgcgaggtgattcgcacacaggtgatgcgggcccctcgtgaaccaaaagcagacacttacgacagtgaggggcgtggttacgtggtgcccatgactgcccaggaaaagcacacaggctgggcagtgcacattggaaatggccgactggtcacttgcacacatgtggccaacatgtgcgatcgcgtagctgaggtggagttcaagatcacggaaacagatcgcgacacttgcatcatcactgccccactcggccacctacccagtgttgcccttggggatggaccaccggccttttacaccaccaatttccaccccatccgtgtgttggatgagggcagttgggacaccacaaccacccgtgttactggttggcgtgttgttatcaataacggcaccacaactgcccctggtgactgcggccaaccatacctgaacgcacgccgacagctcgttggtgtacatgcagcaacaagtacatgtggggtcaagaaactggtgtctagggtgcagaccaagagagcatcaaaggccacattcccttggaagggcctgcctgtcaccaatatgcccgatgctggcggcttgcccactggcacgcggtaccaccgctccatcgcgtggcccaagcagttacctgaggaaacgcacgcccccgccccctatggtgtcaatgatccccggtaccccttctcccaacaccaaatgattgccaacaacctacaaccctacaccaaaaccccagtcgcgctagaccaaaccttgctgcaacgtgctgttaaacacacaaagggttacctagaccaagttattggaacacacagatccccaaacctcacctatgcggcagcagtggattcgatggcccacgacacagcatgtgggccaaatctacctggccgcaagaaggattacatgacagatcaaggggagcccattggaccactcaaagagatgttggaggaagcttgggacatggctcaccgaggtgtcccgcgcaagcacgagtacaagcttgcactcaaggatgaactccggcccatagagaagaatgatcaaggcaagaggcgcctcctttggggttgcgatgctggggtgtcactagtggccaacgccgtgtttaagcctgtggccgagcgcctggtggacaccgtgcccatgcaccctgtggctgttggcatatgcatggacagcccccaaattgaacagatgaaccaggccttaactggtagggtgctatactgtttggattacagcaagtgggattccacgcaaaacccagccgtgacaagcgccagtgtcgacattttggcctcgtacgccgaggacacaccactcacatcggccgccatagctacactatgttcaccagccatcggtcgacttgacgacattggcttgactgtcacaacgggtctcccctctggcatgccgttcaccagcgttataaattcagtcaaccacatggtgtattttgccatggctgtgcttgaggcctacgaggagttcaaggtaccctatatgggaaacatttttgacaatgagaccgtttacacctatggggatgactgtgtttatggcctaacaccggcaacagcttccatcatgccggtggtggtcaagaacctgaccagttacggattggttccaaccgctgcagataagtcccaaaccattgaacccactgacaccccagtgtttctcaaacgcaccttttcccaaacaccttttggactgcgggcattgcttgatgaaactagtctggcgcgacagtgctactgggtcaaagccaaccgcacaactgacctctttgagccagcagctgttgatgtggaaattaggaaaaaccagctggaggtcatgctcgcatacgccagccaacatccaagggtggtgtttgacaagcttgcaagcatggttgagacaaccgcaaatgttgagggttaccaagttgtgaatgttgattgggccaacgcagtggcaacgtacaacgcatggtacggtggcactgacggtggccacgctcccaccaatgaagatgaagagccagaagtgttcgtgatggaggcacctgccccaacccgttcggcggcgagcaacccagagggtactcaaaccagcaatgaatccaggccagttcagccagccgggcccatgcccgtggccactgcccaggcccttgagatggctgttgccactggacaagtcaatgacaccatccccagtgtggttagagaaactttcagcacctacaccaatgtcacctggactacacgtcagccagcaggaaccctgctcgcccggatgaccctagggccaggcctgaatccctacacactccacctgtccgccatgtgggccggctggggagggtcatttgaaatcaaggtggtaatatcggggtctggtttgtatgcaggaaaattgctgtgcgcactcataccacccggggtggaccccagtgctgtagaccagcctggagcttttccccatgcgcttgtggatgcgcgcatcactgagggtgtcaccttcaccctcggggacgtcagggcagtggactaccacgagacaggagctggcgggaccatcgcttgcttggcactctacgtgtaccaaccactcatcaacccttttgaaactgctttgtcagccgccatggtgacaatcgagacccgccccggcccagactttgggttcaccctgctcaagcctccaaaccaaaccatggaggctggacttgaccccagatcgctcctgccccgcacggcaaggacactgcggggaaacaggtttggcaggcccatcacggccgtggtcatagttggcctagcacaccaaatcaacaggcacttctcagccgagggcaccacgctcggttggtccacagccccaattggtccttgtgtgggtcgcatcaattccaggtacaccaacaatggcggccttgctgtgctctcgatgcagcccctgagcaatgggcccctctaccccaacatcatcaaccactacccagacgtggctgcatcaagggcattcaacacaaacactaccctgaacaacgacaccacgtgtgggggggggcccatggtgatcttcaatgatgtgggtgatgtggttgaaactgtgtcctaccaaatgagattcatagcctcacaagccacctcccaaacacccacacttgttgattacatcaatgcaacatcaatgggactgtgcagctttggcaactctcggggggactttggctcaggccagctcaatgtgggcgttgaactgacctacacctgtggcaacacagcgatcaatgagaaagtcaccacattcatggatcgccaatacacatttggtgcacaggggccaaataacatcatgctctgggtggagactgtgctcggcacgcacacgggcaataacactgtgtatagctcgcaacccgacactgtgtctgccgcactgcagggtcaaccttacaatataccagatgggtacatggctgtgtggaatgttaatgcaaacagtgccgatttccagataggcctaaggcgcgatggcttctttgtcaccagtggggccattggtacgcgcatggtcatctcagaggacaccaccttcacctacgctggcattttcaccctcactacccccctcattggaccaagtgggatgacaggacggtcccttcacagctcacgatgagttgggttgcaggtgcaatgcagggtgctggcctgctaggtgacttggcaggcacgattggacagatagtgctttacaacaagcagttgaacattcagaaatcttttaatcaggcccagatggagttggccaaagaacaactaaaacaaaatcaacaaatggccaaccagttttatgattttaattctaatttacctgctaatcagtacaacagtgctgtgggtgctggctttgactcggtctctgcacgtcaattggctgggtcacacgaagtgcgctactttggtggacaacaaacacctctgctccaccaaggacagatgcatcaaatgatgttctcatcaaaacacttgatgcaagcacaaaacgtgcttggcactttttcccgtgggacacctggtgttacccccagcaaagggttgcctaaaccgcaaggcatcacccccaggccacagggcgtcacagcctccgtgcccgtggctggagtcaccaccactcacagcaaagtgtgattttctttccctggttacgcgtaccgaaccttacacagtgtggcggctgtgggg

SEQ ID NO: 22 - RNA, 7349 nt, Sapporo virus
  source 1..7349: mol_type = genomic RNA
gtgatcgtgatggctaactgccgtccgttgcctattgggcgcttgcccaacaggatttttagcaccccacgactcaccccagggtgggtgtgggcgtgcactaagaaagccatgttcaagccagagtggcttcaggatccggtggtcattcgacagccggatgtttacaccgcgcaaggggtggtggatgacttcttccgccccaagcgtgttttacagggtgatccacaactcatagcacaggtgttgcttggtgatgccagcggcccgctggtcggacctgtatcaatgcagcaactaacatcgttgctgcatgaggtttcacaggcactaaatgaccacaagcatcccctggccaatagatacaccaaggcctctctacaaagatatgcggacaccttgtccaactacatacccctggtggagatattaactggaccaaaggacctcaccccgcgcgacgtgctcgagcaactcgcagctgggcgtgaatgggagtgtgtcccagacagtgccctcaagaaggtctttcgggacatgtggcaatacatttgtgagggctgtgatagcgtgtacatcaaactgcaggatgtgaagcgcaaaataccccacattgacaccactgtgttgaaacagttcttcatcacactcacagacaccatatcaatggcaacagcgctagacaccaaaacctggttggcacacatactgggttggcttaagcccacctgtttggtgatgattatgcaacaacacatcaactcaccccaggggtgggcggccacactaactgccctcgcagagctctactatggtatcatgccgctcaccgaggtgctcgggtccatcgccggttgggttaccgacaagtttgctgatgtaaccaccaacacctggggcaagttcaaggcttggtgggacaatctttacacaccgcaagctggcaatgacttgatcatactaggtggtgttgtcgggctggtgtacttcatggtttttggagatgcacccacccagatgttcaccaagaagctaatgaaggtgtgtgggttcatcacatccactgtggccgcaatcaaggctgctatgtggatagttgattacttcaagcaacgcgaacatgaacatcaggttcgcgtgaccttggccaggtgggccgcactccaggaagtcatcaagcagaacaggtgtgctggccttagtgaagtcaccaaactgaaagaatgctgtgaagtcttactgaacgaggtcaccgaattgatgtacaagctgggcgcctcgcctctagcggggttgatacgcagcacctctgatgtcatacagaccaacatcaacgacttggctcagctgatggcgtacgacacccaacgcaaaccccccgccatgatcgtgtttggtgggccaccaggcataggtaagacccggctggttgaagctctggccagacagcttggggaggtcagccacttcaccatgaccgttgaccactacgatacctacacaggtaatgcagtggcaatatgggacgaatttgatgtggattcaaagcaggcattcatcgaggccaccatcggtattgttaactgtgccccctaccccttaaactgcgaccggccagaggcaaagggcagggtattcacatcaaagtatgtgttggccaccaccaactgtcccacaccagtcatgcccgatcacccgagggctatggcgttctggcgccgcataaccttcattgacgtcactgcccccacaattgaacaatggcttgttgacaaccccgggcgcaaggcaccgcctagccttttcaaagatgacttttcccacttgcagtgttccgtccgcgggtacactgcctatgatgaaaaaggcaacacgctcagtggcaaggtggctcgggccaggtacgtgtcagtcaataacctgctcgacctgatcaaagagaaatacaacagtgaagccactgacatcaaacacctgtggttcaccgttccgcagggcattcacaagcaagcacgagatatcatactggggtggctgcggttccactcctaccccaacaccgtggctgacaacatacccctttcagaagtcagggatcccacctgcttcgggtatgtagtcatttctgatgtggacccacctcggcacgtgagtgaccatgtggcacacattgaagtggagtcaatattgcgcaccgacatcatggaccttttgagagagggaggcggaggcctgttcagggcgttgaaggttaaaagtgccccccgaaattgtgtgatcaacaaagtcatgatgcaagcgcaccacaccaccctgcaggtgctcaccagccaggaaccaaacccaccaaacctgccccgacctagacgcttggtttttgttgagtcaccaattgacatcattagcgccctgcgacaccacgtggggttctgcaccattccaggcatagtcaagttgatcacatcaggagtgggcctgggagtggaaaacctgggcaatttcttgcagtccattgccggcaacgtcagattccctttgcaatccgagtgctcactgctcaggacacctggcggtgacgtgctattttacacctcagggcaagcagcagtgtgggcgacacccgcacgttttccaatcgtcactccgggagaggcatctgttggcaaagatgtgtgttctgagtcctcctggtgggacatcctcaaggccctgtttagcacgctggtggtggccttcggccccatagccacactggtgctgaccgcacacaacctggcctacctgaacacccgtgaaaacaccttgagtgaggcaaaaggtaaaaacaagcgtggccgcggtgcccgcaaaaccatcgccctcagggacgatgagtatgatgaatggcaggacatcattcgtgactggcgcaaggaaatgactgtccaacagtttttagatctgaaagaacgcgccctttccggcgcctctgatcctgattcgcagaggtacaatgcctggctggaactgcgcgcaaagagattgagtgccggggcctaccagcatgcagttgtggacatcattgggaagtcaggacataggcgtgaggtgattcgcacgcagatgatgcgggcgccccgtgaaccaaaagcggacacctatgatagtgaggggcgtggttacgtggtgcccatgactgcccaggaaaaacacacaggctgggcagtgcacattggaaatggccgactggtcacttgcacacatgtggccaacatgtgcgatcgcgtggctgagatggagttcaagatcacagaaacggatcgcgacacttgtattatcaccgccccactcggccacctacccagtgttgcccttggggacggaccgccagccttttacaccactaatttccaccccatccgtgtgttggatgagggcagttgggacaccaccaccacccgtgtcaccggttggcgtgttgtcattaacaacggcaccacaactgcccctggtgattgcggccaaccgtacttaaacacacgccggcagctcgttggcgtgcatgctgcaacaagcacatgtggggtcaagaaactggtatccagggtgcagaccaaaagggcatcaaaggccacgttcccttggaagggcctgcctgtcgccaacatgcccgatgctgggggcctgcccactggcacgcggtaccaccgctctgtcgcgtggcccaagcagttgcctgaggagacgcatgcccctgccccctatggtgtcaatgatccccggtaccccttctctcaacaccagatgattgccaacaacctacaaccctacaccaagaccccagtcgcattagaccagaccttgctacaacgtgctgtcaaacacacgaagggttacctggaccaagtcatcggaacacacagatccccaaacctcacctacgcggcagcagtggactcgatggcccacgacacagcgtgtgggccaaatttatctggccgcaagaaggactacatgacagatcagggggaacccattggaccactcaaacaaatgttagaggaggcttgggacatggctcaccggggtgttccgcgcaaacacgagtacaagctcgcgctcaaggatgaactccggcccatagaaaagaatgatcaaggcaagaggcgcctgctttggggctgtgacgctggggtgtcgctggtggccaacgccgtgtttaaacctgtagccgagcgcttggtggacaccgtgcccatgcaccctgtagctgttggcatatgtatggacagcccccaaattgagcagatgaaccaggccctaactggtagggtgctgtactgcttggactacagcaagtgggactcaacgcagaacccagctgtgacaagcgccagtgttgacatcctggcctcgtacgctgaggacacaccactcacatcagctgccatagccacactgtgttcgccagccatcggtcgacttgacgacattggtttgaccgtcacaacgggtctcccctctggcatgccgttcaccagcgtcataaattcagtcaaccacatggtgtattttgccatggctgtgcttgaggcttatgaggagttcaaggtgccctacatggggaacatttttgacaatgagactgtttacacctacggggatgactgtgtgtatggcctgacaccggctacagcttccatcatgccggtggtggtcaaaaacctgaccagttacggactggttccaactgctgcagacaaatcccaaaccattgaacccactgacaccccagttttcctcaaacgcaccttttcccagacaccctttggactgcgggcactgcttgatgaaactagtctggcgcgacagtgctactgggttaaagctaaccgcacaacagacctttttgagccagcagctgttgatgtggagattaggaaaaaccagttggaggtcatgctcgcgtacgccagccagcatccaagggcagtgtttgacaagcttgcaaacatggttgaggcaactgcaaatgctgagggttatcaagttgtgaatgttgattgggccaacgcagtggcaacgtacaacgcatggtacggtggcactgacggtggccacgctcccaccaatgaagatgaggagccagaagtgttcgtgatggaggcgcctgccccaacccgtccggttgcgagcaacccagaaggcactgagaccagtgatgagtccaggccagttcagccgcctgggcctgtgcctgtggccgcagcacaggctctggagatggccgttgccacagggcaaatcaatgacaccatccccagtgtggtcaggcaaacattcagtacttacaccaatgttacctggaccacgcgtcaacccgcaggaaccctgcttgcccgaatgtccctagggccaggattgaacccttacacgcttcatttgtccgccatgtgggccggttggggtgggtcgtttgatattaaggtggtgatatcagggtctggcatatatgcaggcaaactgctgtgtgcactcataccacctggggttgaccctagcactgtggatcaacccggggcctttccccacgcgcttgtggatgcgcgcaccactgatggtgtttcatttaacttgggggatgtcagggcagtggattaccatgaaacaggtgctggtggaaacgtggcatgcttggcactttacgtgtaccagccgctcatcaacccctttgaaaccaccacatctgctgccatggtgacagtcgagacccgtcctggcgcagactttgggtttaccctactcaagcctccaaaccagaccatggaggtgggacttgaccccaggtcgctcctgccccgcactgcaagaacactgcgggggaacaggtttggcaggcccatcagatctgtgattatagtaggtttggcacaacaaatcaacaggcatttctccgcagaaggcaccacacttggttggtccacggccccaattggcccctgtgcaggccgcatcaacaccaagtacactggtagtgcgggcaaggtggtggctcaactgttacctttgagcaatgggcccctttacccaaacatcatcaaccactacccagacgtggctgcatcaacaactttcagtggagggtctagcataacccctgacatgacgtgtgggggaggacccatggtgctcttcaacgatgtgggtgatgtggtggagaccgtctcctaccaaatgaggttcatagcctcacaggccacatctcaaagtaccacactcaccgacaagatcaatgcaacatcaatgtcagtggtcagttttgacagctcccggaatgacttcccccaatcaaatgacaatgtgggtattgagttaacctacacttgtggcaacacaccgatcaatgggaacgtcacccagttcatggaccgccaatacacctttggcgcacaggggcccaacaacatcatgctttgggtggagtctgtgcttggcacgcacactggcaacaacagagtctacagctcacaaccagataccgtgtctgctgcactgcagggccagccctacaacataccacaggggtacatggccgtgtggaatgtcaacgcggacagtgctgactttcaaatagggctgaggcgcgatggctactttatcaccaatggagccattggcacagaaatggctatttcagatgacaccaccttcaccttcaatggaatgtacaccctaaccactcccctcattggaccaagtgggacaactggaaggtccgttcacagctcacgatgagttgggttgcaggcgcaatgcagggcgctggcctgctgggtgacctcgcgggcacaattggacaaatagtgttgtacaataagcagttgaatattaccaaatcttttaatcaggctcagttagagttagctaaagatcaaatgaaacaaaatcaacagttggctaaccagtattatgaatttaattcaaatttgcctgccaatcagtacaatagtgctgtgagtgctggttttgacgcggtttctgcacgtcaattagctgggtcgcacgaagtgcgctactttggtggacaacaaacgccattgctccaccaagggcaaatgcaacaaatgatgttttcatcaaagcatctcatgcaagcgcaaaacgtgcttggcaccttttctcggggaacacctggtgtcacccccagtaaggggctgcccaaaccacagggcgtcacgtccagaccgctcagaccacagggtgtctcagccaccatacctgtggccggagccaccaccaccaacagcagagtgtgattttctttccctggttacgcgtaccggaccttacacagtgtggcggctgtgggg

SEQ ID NO: 23 - RNA, 7340 nt, Sapporo virus
  source 1..7340: mol_type = genomic RNA
gtgatcgtgatggctaactgccgtccgttgcctattgggcacttgcccaacaggatttttagcaccccacgactcaccccagggtgggtgtgggcgtgcaccaaggaagccatgtttaagctagagtggcttcaagatccggtggtcattcgacagccggatgtttacatcgcgcaaggggtggtggatgacttcttccgccccaagcgtgtcttacagggtgatccacagctcatagcacaggtgctgcttggtgatgccagcggcccgctggtcgggcctgtttcaatgcagcaactaacatcgttgctacatgaggtttcacaggcactaaatgaccacaagcatcccctggccaacagatacaccaaagcctctttgcaaagatatgcagacaccttgtccaactacatacctctagtggagatattaactggaccaaaggacctcaccccgcgtgacgtgctcgaacaactcgcggctgggcgtgaatgggagtgtgtcccagacagcgccctcaagaaagtttttcgggacatgtggcaatacatttgtgagggctgtgacagcgtgtacatcaaattgcaggatgtgaagcgcaagataccccacattgacaccaccgtgttgaaacagttctttattacactcacagacaccatatcaatggcaacagcgctagacaccaaaacctggttggcacacatactgggttggctcaagcccacctgtttggtgatgatcatgcaacaacacatcaactctccccaggggtgggcggccacactaactgcccttgcagagctctactatggcatcatgccgctcaccgaggtgctcgggtccattgccggttgggtcactgacaagtttgctgatgtgaccaccaacacctggggcaagttcaaagcttggtgggacaatctttacacaccgcaagccggcaatgacttgatcatactaggtggtgttgtcgggctggtgtacttcatggtttttggagatgcacccacccaaatgttcaccaagaagctgatgaaggtgtgtgggttcatcacatccactgtggcagcgatcaaggctgccatgtggatagttgattattttaagcaacgcgagcatgaacatcaggttcgcgtcaccttggccaggtgggccgcactccaggaggttatcaagcagaataggtgtgctggccttagtgaaatcaccaaactgaaggagtgctgtgaagtcttgctgaatgaggtcaccgaattgatgtacaagctgggtgcctcgcctctagcggggttgattcgtagcacctctgacgtcatacaaaccaccatcaatgatttggctcagctcatggcgtacgacacccaacgcaagccccccgccatgatcgtgtttggtgggccacctggcataggtaagacccgattggttgaagctctggccagacagcttggggaggtcagccacttcaccatgaccgtcgaccactatgacacctacacaggcaatgcagtggcaatatgggacgaatttgatgtggactcaaagcaggcattcatcgaggccaccatcggtattgtcaattgtgccccctaccctttaaactgcgaccggccagaggcaaagggcagggtgttcacgtcaaagtatgtgttggccaccaccaactgtcccacaccagtcatgcctgaccacccgagggccatggcgttctggcgtcgcataaccttcattgatgtcactgcccccacaattgaacaatggttggttgacaaccccgggcgcaaggcaccggctagcctttttaaagaggacttctcccacctgcagtgctccgtacgtgggtacacagcctatgacgaaaaaggcaacacgctcagtggcaaagtggctcgggctaggtacgtatcagtcaacaacctgctcgacctgatcaaagagaagtacaacagtgaagccactgacattaaacatctgtggttcaccgttccacagagcatccacaagcaggcacgagacatcatactggggtggctgcggttccactcctaccccaataccgtggctgacaacatacccctttcggaagtcagggaccccacctgcttcgggtatgtggtcatctctgacgtggacccgccccggcatgtgagtgaccatgtggcacacattgaagtggagtcaatactgcgcaccgacatcatggaccttttgagagagggaggcggaggcctgttcagggcgttgaaggtgaaaagcgcccctcggaattgtgtgatcaacaaagtcatgatgcaagcgcaccacaccaccctgcaggtgcttaccaaccaggaaccaaacccaccaaacctgccccgacctagacgtttggtttttgttgagtcaccaattgacatcatcagcgccctgcgacaccacgttgggttctgcaccattccaggcatagtcaagttgatcacctcgggagtgggcctgggtgtggaaaacctgggcaacttcttgcaatccatcgccggcaacgtcagattccctttgcagtccgagtgctcactactcaggacacccggcggtgatgtgctattctacacctcagggcaagcagcagtgtgggcgacacccgcacgttttccaatcatcactccgggagaggcgtctgttggcaaagatgtgtgttctgagtcctcctggtgggacatcctcaaggccttgtttagcacgctggtggtggccttcggccccatagccacattggtgctgactgcacacaacttggcctacttgaacacccgtgagaacaccttgagtgaggcaaaaggtaaaaacaagcgaggtcgcggtgcccgcaaagccatcgccctcagggacgatgagtatgatgagtggcaggacatcattcgtgactggcgcaaggaaatgactgtccaacagtttttagacctgaaagaacgcgccctttccggtgcctctgaccccgattcgcagaggtataatgcctggttggaactgcgcgcaaaaaggttgagtgccggggcctaccagcatgcagttgtagacatcattgggaaatcaggacacaggcgcgaggtgattcgcacacaggtgatgcgggcgccccgtgaaccaaaggcagacacctacgatagtgaggggcgtggttacgtggtgcccatgactgcccaggaaaaacacacaggctgggcagtgcacattggaaatggccggctggtcacttgcacacatgtggccaacatgtgcgatcgcgtggctgaggtggagttcaagatcacagaaacagatcgcgacacttgcatcatcactgccccactcggccacctacccagtgttgcccttggggatggaccaccggccttttacaccaccaactttcaccccatccgcgtgttggatgagggcagttgggataccaccaccacccgtgtcaccggttggcgtgttgtcattaacaatggcaccacaacagcccctggtgattgcggccaaccgtacttgaacgcacgccggcagctcgttggtgtgcatgcagcaacaagcacatgtggggttaagaaactggtgtctagggtgcagaccaaaagggcatcaaaggccacattcccatggaagggcctgcctgtcaccaacatgcccgatgccgggggcttgcccactggcacgcggtaccaccgctccatcgcgtggcccaagcagttgcctgaggagacacatgcccctgccccctatggcgtcaatgatccccggtaccccttctcccaacaccaaatgatcgccaacaacctacagccctacaccaaaaccccagtcgcactagaccagaccttgctgcaacgtgctgttaaacatacaaaggggtatctagaccaagtcattggaacacacagatccccaaacctcacctacgcggcagcagtggactcgatggcccatgacacagcatgtgggccaaatttgcctggccgcaagaaggattacatgacagatcagggggaacccattggaccacttaaacaaatgttggaggaagcttgggacatggctcaccgaggtgtcccgcgcaagcacgagtacaagcttgcgctcaaggatgaactccggcccatagaaaagaatgatcaaggcaagaggcgcctcctttggggctgtgacgctggggtgtcgctggtggccaacgccgtgtttaaacctgtggccgagcgcctggtggataccgtacccatgcacccggtggctgttggcatatgcatggacagcccccagattgaacagatgaaccaggccttaactggtagggtgctgtactgcctggactacagcaagtgggactccacgcaaaatccagctgtgacaagcgccagtgttgacatcttggcctcatacgctgaggacacaccactcacatcagctgccatagctacactgtgttcgccagccatcggtcgacttgacgacattggcttgactgttacaacgggtctcccctctggcatgccattcaccagtgtcataaattcagtcaaccacatggtgtattttgccatggctgtgcttgaggcttacgaggagttcaaggtgccctacatggggaacatttttgacaatgagactgtttacacttacggggatgactgtgtgtatggcctcacaccggcaacagcttccatcatgccggtggtggtcaaaaacctgaccagttacgggctggttccaactgctgcagacaaatcccaaaccattgaacccactgacaccccagttttcctcaaacgcaccttttcccaaacaccctttggactgcgggcactgcttgatgaaactagtctggcgcgacagtgctactgggttaaagctaaccgcacaacagacctttttgagccagcagctgttgacgtggaaatcaggaaaaaccagttggaggtcatgctcgcgtacgccagtcagcatccaaggatagtgtttgacaagcttgcaagtatggttgaggcaactgcaaatgccgagggctaccaagttgtgaatgttgattgggccaacgcagtggcaacgtacaacgcatggtacggtggcactgacggtggccacgctcccaccaatgaagatgaagagccagaagtgttcgtgatggaggcgcctgccccaacccgttcggtggcgagcaacccagagggtacccaaaccagcaatgaatccaggccagttcagccagccgggcccatgcccgtggccactgcccaggcccttgagatggctgttgccactggacaagtcaatgacacaatccccagtgtggtcagagaaactttcagcacctacaccaatgtcacctggactacacgtcagccagcgggaaccttgctcgcccggatgaccctagggccaggcctgaacccctacacactccacctgtctgccatgtgggctggctggggagggtcatttgaaattaaggtggtgatatcggggtctggcttgtatgcaggcaaattgctgtgcgcgctcataccacctggggtggaccccagtgctgtggaccagcccggggcttttccccatgcacttgtggatgcgcgcatcactgagggcgtcaccttcacccttggggatgtcagagcagtggattaccacgaaacaggagctggtgggaccatcgcttgcttggcactttatgtgtaccaaccactcatcaacccctttgaaactgctttgtcagccgccatggtgaccatcgagacccgccctggcccagactttgggttcaccctgcttaagcctcctaaccaaaccatggaggcaggacttgaccccagatcgcttctgccccgcacagcaaggacactgcggggaaacaggtttggcaggcccatcacaggcgtggtcatagtcggcatggcacaccagatcaacaggcacttctcagccgcgggcaccacgcttggctggtccacagccccaattggtccttgtgtaggacgtattaattctaggtacaccaacaacggcggccttgccgtgctctcgttgcaacccctaagcaatgggcccctctaccctaacatcatcaaccactacccagatgtggctgcttcaaaagctttcaacacaagcactaccctgagtgatagcaccacgtgtggtgggggacctatggtgattttcaatgatgtgggtgatgtggttgagaccgtgtcctaccaaatgagattcatagcctcacaagccacctcccaaacacccacactcgttgattacatcaatgcaacatcaatgggattggtcagttttggcaattctcggggggactttggctcaggccagctcaatgtgggcgtcgagttgacctacacctgtggcaacacagcgatcaatgagaaagtcactacgttcatggatcgccaatacacatttggtgcacagggtcccaacaacatcatgctctgggtggagactgtactcggcacgcacacgggcaacaacactgtgtacagctcgcaacccgacactgtgtctgctgcactgcagggtcagccatacaacataccagatggatacatggctgtgtggaatgttaatgcaaacagtgccgatttccagataggcttgaggcgtgatggcttctttgtcaccagtggggccattggcacgcgcatgaccatctcagaggacaccaccttcacctacgccggcatgttcaccctcaccacccccctcattggaccaagtgggatgacaggacggtcccttcacagctcaccatgagttgggttgcaggtgcaatgcaaggtgctggcctgctaggtgatctagcaggcacaattggacaagtagtgctctacaacaagcagttgaatatccagaagtcttttaatcaagcccagatggagttggccaaggaacaattgaaacaaaaccaacagatggccaaccagttttatgattttaattcaaatttacctgctaatcagtacaatagtgctgtgggtgctggctttgactcggtctctgcacgtcaactggctgggtcacatgaagtgcgttattttggtggacagcaaacacctttactccaccaagggcaaatgcaccaaatgatgttttcatcaaaacacctgatgcaagcacaaaatgtgcttggcactttttcccgtggaacgcctggtgtcacccctagcaaaggactgcctaaaccacagggcatcaaccccaggccacagggcgtcacagcctccgtgccggtggctggagccaccaccactcacagcaaagtgtgattttctttccctggttacgcgtaccgagccttacacagtgtggcggctgtgggg

SEQ ID NO: 24 - RNA, 7340 nt, Sapporo virus
  source 1..7340: mol_type = genomic RNA
gtgatcgtgatggctaactgccgtccgttgcctattgggcacttgcccaacaggatttttggcaccccacgactcaccccagggtgggtgtgggcgtgcactaaggaagccatgtttaagctagattggcttcaagatccggtggtcatccgacagccggatgtttacatcgcgcaaggggtggtggatgatttcttccgccccaagcgtgttttacagggtgatccacagctcatagcacaggtgttacttggtgatgccagcggcccactggtcgggcctgtctcaatgcagcaactaacatcgttgctgcatgaggtctcacaggcactgaatgaccacaagcaccccctggccaacagatacaccaaagcctctttgcaaagatatgcagacaccttgtccaactacatacctctagtggagatattaactggaccaaaggacctcaccccgcgtgacgtgctcgagcaactcgcggctgggcgtgaatgggagtgtgtcccagacagtgccctcaagaaggtctttcgggacatgtggcaatacatttgtgagggctgtgacagcgtgtacatcaaactacaggaagtgaagcgcaagataccccacattgacaccaccgtattgaaacagttctttatcacacttacagacaccatatcaatggcaacagcgctggacaccaagacctggttggcacacatactgggttggctcaagcccacctgtttggtgatgattatgcaacaacacatcaactccccccaggggtgggcagccacactaactgcccttgcagagctttactacggcatcatgccgctcaccgaggtgctcgggtccatcgctggttgggtcactgacaagtttgctgacgtgaccaccaacacctggggcaagttcaaggcttggtgggacaatctttacacaccgcaggctggcaatgacttgatcatactaggtggtgttgtcgggctggtgtacttcatggtttttggagatgcacccacccagatgtttaccaagaagctaatgaaggtgtgtgggttcatcacatctactgtggcagcgatcaaggctgccatgtggatagttgattactttaagcaacgcgagcacgaacaccaggttcgcgttaccttggccaggtgggccgcactccaggaggtcatcaagcagaacaggtgtgctggccttagtgaaatcgccaaactgaaggagtgctgtgaagtcttgctgaatgaggtcaccgaattgatgtataagctgggcgcctcgcctctagcggggctgattcgtagcacctctgatgtcatacagaccaccatcaatgatttagcccagctgatggcgtacgacacccaacgcaagccccccgccatgatcgtgtttggtgggccacctggcataggcaaaacccggttggttgaagctctggccagacagcttggggaggtcagccatttcaccatgaccgtcgaccactacgacacctacacaggcaacacagtggcaatatgggacgaatttgatgtggactcaaagcaggcatttatcgaggccactatcggtattgtcaattgtgccccctaccccttaaactgcgaccggccagaggcaaagggcagggtgttcacgtcaaagtatgtgttggccaccaccaactgtcccacaccagtcatgcctgaccacccgagggccatggcgttctggcgtcgcataaccttcatcgatgtcactgcccccacaattgaacaatggctgattgacaaccccgggcgcaaggcaccagccagccttttcaaggaggacttctcccacctgcagtgctccgtgcgtggttacacagcctatgacgaaaaaggcaacacgctcagtggcaaggtggctcgagctaggtatgtgtcagtcaataacctgctcgacctcatcaaagagaagtacaacagtgaagccactgacatcaaacacctgtggttcaccgtcccacaaagcatccacaagcaggcacgagacatcatactggggtggctgcggttccactcctaccccaacaccgtggctgacaacatacccctttcagaagtcagggaccccacctgcttcgggtatgtggtcatttctgacgtggacccgcctcgacatgtgagtgaccacgtggcacacattgaagtggagtcaatattgcgcaccgacatcatggaccttttgagagaggggggcggaggtctgttcagagcattgaaggtcaaaagcgcccctcggaactgtgtgattaataaagtcatgatgcaagcgcaccacaccaccctgcaagtgcttactagccaggaaccaaacccaccaaacctgccccgacccagacgtttagtttttgttgagtcaccaattgacatcattagcgccctgcgacatcacgtggggttctgcaccattccaggcatagtcaagttgattacatcgggagtgggcctgggcgtggaaaacctgggcaatttcttgcaatccatcgccggcaatgtcagatttcctctgcagtccgagtgctcactactcaggacacccggcggtgatgtgctattttacacttcagggcaagcggcagtgtgggcgacacccgcacgttttccaatcatcactccgggagaggcgtctgttggcaaagatgtgtgttctgagtcctcctggtgggacatcctcaaggccttgttcagcacgctggtagtggccttcggccccatagcaacattggtgctaactgcacacaacttggcctacttgaacacccgtgagaacaccttgagtgaggcaaaaggtaaaaacaagcgtggtcgcggtgcccgcaaagccatcgccctcagggacgatgagtatgatgaatggcaggatatcattcgtgactggcgcaaggaaatgactgtccaacagttcttggacctgaaagagcgtgccctttccggtgcctctgaccctgactcgcagaggtacaatgcctggctggaactccgcgcaaagaggttgagtgccggggcctaccagcatgcagttgtggacatcattgggaaatcaggacataggcgcgaggtgattcgcacacaggtgatgcgggcgccccgtgagccaaaagcagacacctatgacagtgaggggcgtggttacgtggtgcccatgactgcccaggaaaaacacacaggctgggcagtgcatattggaaatggccgactagtcacttgcacacatgtggccaacatgtgcgatcacgtagctgaggtggagttcaagatcacagaaacagatcgcgatacttgcatcatcactgccccgctcggccacctacccagcgttgcccttggggatggaccaccggccttttacaccaccaacttccaccccatccgtgtgttggacgagggtagctgggacaccaccaccacccgtgttaccggttggcgtgttgtcatcaacaacggcaccactactgcccctggtgattgcggtcaaccgtacttgaacgcacgccggcagctcgttggtgtgcatgcagcaacaagtacatgtggggtcaagaaactggtgtctagggtgcagaccaaaagggcatcaaaggccacgtttccttggaagggcctgcctgtcaccaacatgcccgatgccgggggcttgcccactggcacgcggtaccaccgctctatcgcgtggcccaagcagttgcctgaggagacacatgcccctgccccctatggtgtcaatgatccccggtattccttctcccaacatcaaatgatcgccaacaacctgcagccctacaccaaaaccccagtcgcattagatcaaaccttgctgcaacgtgctgtcaaacacacaaagggctacctagatcaggtcattggaacacacagatccccaaacctcacctacgcagcagcagtggactcgatggcccacgacacagcatgtgggccaaacttgcctggccgcaagaaggattacatgacagatcagggggagcccattggaccacttaaacaaatgttggaggaagcttgggacatggcccaccgaggtgtcccgcgcaaacatgagtacaagcttgcactcaaggatgaactccggcccatagaaaagaatgaccaaggcaagaggcgcctcctttggggctgcgacgctggggtgtcgctggtggccaacgccgtgttcaaacctgtggctgagcgcttggtggacactgtgcccatgcacccagtggctgttggcatatgtatggatagcccccaaattgagcagatgaaccaggccttgactggtagggtgctgtactgcctggactacagcaagtgggactccacgcaaaatccagccgtgacaagcgccagtgttgacatcttagcctcgtacgctgaggacacaccactcacgtcagctgccatagccacactgtgttcgccagccatcggtcgacttgacgacattggtttgactgttacaacgggtctcccctctggcatgccgttcaccagtgtcataaattcaatcaaccacatggtgtattttgccatggctgtgcttgaggcttacgaggagttcaaggtgccctacatggggaacatttttgataatgagactgtctacacctatggggatgactgtgtatatggcctgacaccggcaacagcctccatcatgccggtggtggtcaaaaatctgaccagttacggactggttccaactgctgcagacaaatcccaagctattgaacccactgacaccccaattttcctcaaacgcaccttttcccaaacaccctttgggttgcgggcactgcttgatgaaactagtctggcgcgacagtgctactgggtcaaggctaaccgcacaacagacctttttgagccagcagctgttgatgtggaaattaggaaaaaccaattggaggtcatgctcgcgtacgccagtcagcatccaagggcagtgtttgacaagcttgcaggcatggttgaggcaactgcaaatgccgagggttaccaagttgtgaatgttgattgggccaacgcagtggcaacgtacaacgcatggtacggtggcactaacggtggccacgctcccaccaatgaagatgaagagccagaagtgttcgtgatggaggcgcctgccccaacccgttcggtggcgagcaacccagagggtactcaaaccagcaatgaatccaggccagtccagccagccgggcccatgcccgtggccactgcccaggcccttgagatggcagttgccactggacaagtcaatgacacaatccccagcgtggtcagagaaactttcagcacctataccaatgttacctggaccacgcgccagccagcagggaccctgcttgctcggatgaccctagggccaggcctgaatccctacacactccacctgtccgccatgtgggctggctggggagggtcatttgaaatcaaggtggtgatatcggggtctggcttgtatgcaggcaagttgctgtgcgcactcataccacctggcgtagaccctagtgctgtggatcagcccggggcttttccccacgcacttgtggatgcgcgcatcactgagggtgtcaccttcacccttggggacgttagggcagtggattaccacgaaacaggagctggtgggaccatcgcttgcctggcactttatgtgtaccaaccactcatcaacccctttgaaactgctttgtcagccgccatggtaacaatcgaaacccgccctggtccagactttgggttcaccctgcttaagccccctaaccaaaccatggaggcaggacttgaccctagatcactcctgccccgcacagcaaggacactgcggggaaacaggtttggcaggcccattacaggcgtggttatagtcggcatggcataccaaatcaacaggcatttctcagctgcgggcaccacgcttggctggtccacggccccaattggtccttgtgtgggacgcatcaattctaggtacacaaacaacggcggccttgccgtgctctcgatgcaacccctgagcaatgggcccctttaccccaacatcatcaaccattacccggatgtggctgcttcaaaagcattcaacacaagcactaccctgagtgatagcaccacgtgtggtgggggacccatggtgcttttcaatgatgtaggtgatgtggttgagaacgtgtcctaccaaatgagattcatagcctcacaagccacctcccaaacacccacactcgttgattacatcaatgcaacatcaatgggattggtcagttttggcaattctcggggggactttggctcaggccagctcaatgtgggcgttgagttgacctacacctgtggtaacacagcgatcaatgagaaagtcactacgttcatggatcgccaatacacatttggtgcacaggggcccaataacatcatgctctgggtggagactgtgctcggcacgcacacgggtaacaacactgtgtacagctcgcaacccgacactgtgtctgccgcactgcagggccagccctacaacataccagatggttacatggctgtgtggaatgtcaatgcaaacagtgccgatttccagataggcttgaggcgtgatggcttctttgtcaccagtggggccattggcacgcgcatgaccatctcagatgacaccaccttcacctacgctggcatgttcgccctcaccacccctctcattggaccaagtgggatgacaggacggtcccttcacagctcaccatgagttgggttgcaggtgcaatgcaaggtgctggcctgctgggtgacctggcaggcacaattggacaaatagtgctttataacaagcagttgaatattcagaaatcttttaatcaagcccagatggagttggccaaagagcaattgaaacaaaatcaacagatggccaaccagttttatgattttaattcaaatttacctgccaatcagtacaacagtgctgtgggtgctggctttgactcggtctctgcacgtcaattggctggatcacatgaagtgcgttattttggtggacaacaaacacccttacttcaccaagggcaaatgcaccagatgatgttttcatcaaaacacctgatgcaagcacaaaatgtgctcggcactttttcccgtgggacgcctggtgtcaccccaagcaagggactgcctaaaccgcagggcatcacccccaggccacagggcgtcacagcctccgtgccggtggctggagctaccaccactcacagcaaagtgtgattttctttccctggttacgcgtaccgagccttacacaatgtggcggctgtgggg

SEQ ID NO: 25 - RNA, 6765 nt, Sapporo virus
  source 1..6765: mol_type = genomic RNA
atggctaattgccgtccgttgcctattgggcatttgcccaacaggatttttagcaccccacgactcaccccagggtgggtgtgggcgtgcactaaggaagccatgttccaactagagtggcttcaagaaccggtggtcattcgacagccggatgtttacattgcgcaaggggtggtagatgatttcttccgccccaagcgtgtcctacagggtgatccgcagctcatagcacaggtgttacttggtgatgccagtggtccgctggtcggacctgtctcaatgcagcaactaacatcgttgctgcatgaggtttcacaagcattaaacgaccacaagcatcccctcgccaacagatacaccaaaacctctttacagagatatgcagacaccttgtcaaactacatacccctagtggagatattaactggaccaaaagacctcaccccgcgcgatgtgctcgagcaactcgcggctgggcgtgaatgggagtgtgtcccagacagtgccctcaaaaaggtctttcgggacatgtggcaatacatttgtgagggctgtgacagcgtgtacatcaaactgcaggatgtgaaacgaaagataccccacattgacaccactgtgttgaaacaattcttcatcacactcacagacaccatatcaatggcaacagcactagataccaagacctggttggcacacatactgggctggcttaagcccacttgtctggtgatgatcatgcagcaacacataaactctccccaagggtgggcggccacactaactgcccttgcagagctgtactacggcatcatgccgcttaccgaggtgctcggatccatcgctggttgggtcaccgacaagtttgctgatgtaaccaccaatacctggggcaagttcaaggcttggtgggacaatctttacacaccgcaagccggcaatgacttgatcatactcgggggtgtcgttgggctggtgtacttcatggtctttggagacgcacccacccagatgttcaccaagaagctaatgaaggtgtgtgggttcatcacgtccacagtggcagcgatcaaggctgctatgtggatagtggattactttaagcagcgcgaacatgaacaccaagttcgcgtcactttggcgaggtgggccgcactccaggaggtcatcaagcagaacaggtgtgctggccttagtgaggtcgccaagttgaaagagtgctgtgaagtcttgctgaatgaggtcaccgaactaatgtacaagctgggtgcctcacctttggcggggttgatacgtagcacctctgatgtcatacagaccactatcaatgatctggctcagctaatggcgtacgacacccaacgtaagccccccgccatgattgtgtttggtgggccaccaggtataggcaagacccggttggttgaagccctggccagacagctcggggaggtcagccatttcaccatgactgtcgaccactatgacacctacacaggtaatgcagtggctatatgggatgaatttgacgtggattcaaagcaggcattcatcgaggccactatcggcatcgtcaactgtgccccctaccccttaaactgtgaccggccagaggcaaagggcagggtgttcacgtcaaagtatgtgctggccaccaccaactgtcccacaccagttatgcctgaccatccgagggctatggccttctggcgccgcataaccttcattgatgtcactgcccccacaattgaacagtggctggttgacaatcccgggcgcaaggcaccggccagcctttttaaagaggatttttcccatttgcagtgttccgtgcgtgggtacactgcctatgacgaaaagggcaacacgctcagtggcaaggtggctcgggccaagtacgtgtcagtcaacaacctgcttgacctgatcaaagagaagtacaacagtgaagccactgacatcaaacacctatggttcactgtcccacagggaattcacaagcaggcacgagacattatactgggttggctgcggttccactcctaccccaacactgtggccgacaacatacccctctcagaagtcagggaccccacctgcttcgggtatgtggtcatatctgatgtggacccgcctcggcacgtgagtgaccatgtggcacatattgaagtggaatcaatattgcgcaccgacatcatggacctcctgagagagggaggtggtggcctgttcagggcgttgaaggtcaaaagcgccccgcggaattgtgtgattaacaaagtcatgatgcaagcacaccacaccaccctgcaggtgcttacaagccaggaaccaaacccaccaaacttgccccgacccaggcgcttggtttttgttgagtcacccattgacatcattagcgccctgcgacaccacgtggggttctgcaccatcccaggcatagtcaagttgattacatcgggagtgggcctgggtgtagaaaacttgggcaattttctgcagtccatcgccggcaacgtcaggtttcctttgcagtccgagtgctcactactcaggacacccggcggtgatgtgttattctacacttcagggcaagcagcagtgtgggcgacacccgcacgttttccaatcgtcactccgggagaggcatctgttggcaaagatgtgtgttctgagtcctcctggtgggacatcctcaaggccttgttcagcacgttggtggtggccttcggccccatagccacattggtgctaaccgcgcacaacttggcctacttgaacacccgtgagaacaccctgagtgaggcaaaaggtaaaaacaaacgtggtcgcggtgcccgcaaggccatcgcccttagggacgatgagtatgatgaatggcaggacatcatccgtgactggcgcaaggaaatgactgtccaacagttcttggacttgaaagagcgcgccctttccggtgcctctgaccctgactcccaaaggtataatgcttggctggaactgcgtgctaagagattaagtgccggggcttaccaacatgcagttgtggacatcatcgggaaatcaggacacaggcgcgaggtgattcgcacacaggtgatgcgggcccctcgtgaaccaaaagcagacacttacgacagtgaggggcgtggttacgtggtgcccatgactgcccaggaaaagcacacaggctgggcagtgcacattggaaatggccgactggtcacttgcacacatgtggccaacatgtgcgatcgcgtagctgaggtggagttcaagatcacggaaacagatcgcgacacttgcatcatcactgccccactcggccacctacccagtgttgcccttggggatggaccaccggccttttacaccaccaatttccaccccatccgtgtgttggatgagggcagttgggacaccacaaccacccgtgttactggttggcgtgttgttatcaataacggcaccacaactgcccctggtgactgcggccaaccatacctgaacgcacgccgacagctcgttggtgtacatgcagcaacaagtacatgtggggtcaagaaactggtgtctagggtgcagaccaagagagcatcaaaggccacattcccttggaagggcctgcctgtcaccaatatgcccgatgctggcggcttgcccactggcacgcggtaccaccgctccatcgcgtggcccaagcagttacctgaggaaacgcacgcccccgccccctatggtgtcaatgatccccggtaccccttctcccaacaccaaatgattgccaacaacctacaaccctacaccaaaaccccagtcgcgctagaccaaaccttgctgcaacgtgctgttaaacacacaaagggttacctagaccaagttattggaacacacagatccccaaacctcacctatgcggcagcagtggattcgatggcccacgacacagcatgtgggccaaatctacctggccgcaagaaggattacatgacagatcaaggggagcccattggaccactcaaagagatgttggaggaagcttgggacatggctcaccgaggtgtcccgcgcaagcacgagtacaagcttgcactcaaggatgaactccggcccatagagaagaatgatcaaggcaagaggcgcctcctttggggttgcgatgctggggtgtcactagtggccaacgccgtgtttaagcctgtggccgagcgcctggtggacaccgtgcccatgcaccctgtggctgttggcatatgcatggacagcccccaaattgaacagatgaaccaggccttaactggtagggtgctatactgtttggattacagcaagtgggattccacgcaaaacccagccgtgacaagcgccagtgtcgacattttggcctcgtacgccgaggacacaccactcacatcggccgccatagctacactatgttcaccagccatcggtcgacttgacgacattggcttgactgtcacaacgggtctcccctctggcatgccgttcaccagcgttataaattcagtcaaccacatggtgtattttgccatggctgtgcttgaggcctacgaggagttcaaggtaccctatatgggaaacatttttgacaatgagaccgtttacacctatggggatgactgtgtttatggcctaacaccggcaacagcttccatcatgccggtggtggtcaagaacctgaccagttacggattggttccaaccgctgcagataagtcccaaaccattgaacccactgacaccccagtgtttctcaaacgcaccttttcccaaacaccttttggactgcgggcattgcttgatgaaactagtctggcgcgacagtgctactgggtcaaagccaaccgcacaactgacctctttgagccagcagctgttgatgtggaaattaggaaaaaccagctggaggtcatgctcgcatacgccagccaacatccaagggtggtgtttgacaagcttgcaagcatggttgagacaaccgcaaatgttgagggttaccaagttgtgaatgttgattgggccaacgcagtggcaacgtacaacgcatggtacggtggcactgacggtggccacgctcccaccaatgaagatgaagagccagaagtgttcgtgatggaggcacctgccccaacccgttcggcggcgagcaacccagagggtactcaaaccagcaatgaatccaggccagttcagccagccgggcccatgcccgtggccactgcccaggcccttgagatggctgttgccactggacaagtcaatgacaccatccccagtgtggttagagaaactttcagcacctacaccaatgtcacctggactacacgtcagccagcaggaaccctgctcgcccggatgaccctagggccaggcctgaatccctacacactccacctgtccgccatgtgggccggctggggagggtcatttgaaatcaaggtggtaatatcggggtctggtttgtatgcaggaaaattgctgtgcgcactcataccacccggggtggaccccagtgctgtagaccagcctggagcttttccccatgcgcttgtggatgcgcgcatcactgagggtgtcaccttcaccctcggggacgtcagggcagtggactaccacgagacaggagctggcgggaccatcgcttgcttggcactctacgtgtaccaaccactcatcaacccttttgaaactgctttgtcagccgccatggtgacaatcgagacccgccccggcccagactttgggttcaccctgctcaagcctccaaaccaaaccatggaggctggacttgaccccagatcgctcctgccccgcacggcaaggacactgcggggaaacaggtttggcaggcccatcacggccgtggtcatagttggcctagcacaccaaatcaacaggcacttctcagccgagggcaccacgctcggttggtccacagccccaattggtccttgtgtgggtcgcatcaattccaggtacaccaacaatggcggccttgctgtgctctcgatgcagcccctgagcaatgggcccctctaccccaacatcatcaaccactacccagacgtggctgcatcaagggcattcaacacaaacactaccctgaacaacgacaccacgtgtgggggggggcccatggtgatcttcaatgatgtgggtgatgtggttgaaactgtgtcctaccaaatgagattcatagcctcacaagccacctcccaaacacccacacttgttgattacatcaatgcaacatcaatgggactgtgcagctttggcaactctcggggggactttggctcaggccagctcaatgtgggcgttgaactgacctacacctgtggcaacacagcgatcaatgagaaagtcaccacattcatggatcgccaatacacatttggtgcacaggggccaaataacatcatgctctgggtggagactgtgctcggcacgcacacgggcaataacactgtgtatagctcgcaacccgacactgtgtctgccgcactgcagggtcaaccttacaatataccagatgggtacatggctgtgtggaatgttaatgcaaacagtgccgatttccagataggcctaaggcgcgatggcttctttgtcaccagtggggccattggtacgcgcatggtcatctcagaggacaccaccttcacctacgctggcattttcaccctcactacccccctcattggaccaagtgggatgacaggacggtcccttcacagctcacgatga

SEQ ID NO: 26 - RNA, 6765 nt, Sapporo virus
  source 1..6765: mol_type = genomic RNA
atggctaactgccgtccgttgcctattgggcgcttgcccaacaggatttttagcaccccacgactcaccccagggtgggtgtgggcgtgcactaagaaagccatgttcaagccagagtggcttcaggatccggtggtcattcgacagccggatgtttacaccgcgcaaggggtggtggatgacttcttccgccccaagcgtgttttacagggtgatccacaactcatagcacaggtgttgcttggtgatgccagcggcccgctggtcggacctgtatcaatgcagcaactaacatcgttgctgcatgaggtttcacaggcactaaatgaccacaagcatcccctggccaatagatacaccaaggcctctctacaaagatatgcggacaccttgtccaactacatacccctggtggagatattaactggaccaaaggacctcaccccgcgcgacgtgctcgagcaactcgcagctgggcgtgaatgggagtgtgtcccagacagtgccctcaagaaggtctttcgggacatgtggcaatacatttgtgagggctgtgatagcgtgtacatcaaactgcaggatgtgaagcgcaaaataccccacattgacaccactgtgttgaaacagttcttcatcacactcacagacaccatatcaatggcaacagcgctagacaccaaaacctggttggcacacatactgggttggcttaagcccacctgtttggtgatgattatgcaacaacacatcaactcaccccaggggtgggcggccacactaactgccctcgcagagctctactatggtatcatgccgctcaccgaggtgctcgggtccatcgccggttgggttaccgacaagtttgctgatgtaaccaccaacacctggggcaagttcaaggcttggtgggacaatctttacacaccgcaagctggcaatgacttgatcatactaggtggtgttgtcgggctggtgtacttcatggtttttggagatgcacccacccagatgttcaccaagaagctaatgaaggtgtgtgggttcatcacatccactgtggccgcaatcaaggctgctatgtggatagttgattacttcaagcaacgcgaacatgaacatcaggttcgcgtgaccttggccaggtgggccgcactccaggaagtcatcaagcagaacaggtgtgctggccttagtgaagtcaccaaactgaaagaatgctgtgaagtcttactgaacgaggtcaccgaattgatgtacaagctgggcgcctcgcctctagcggggttgatacgcagcacctctgatgtcatacagaccaacatcaacgacttggctcagctgatggcgtacgacacccaacgcaaaccccccgccatgatcgtgtttggtgggccaccaggcataggtaagacccggctggttgaagctctggccagacagcttggggaggtcagccacttcaccatgaccgttgaccactacgatacctacacaggtaatgcagtggcaatatgggacgaatttgatgtggattcaaagcaggcattcatcgaggccaccatcggtattgttaactgtgccccctaccccttaaactgcgaccggccagaggcaaagggcagggtattcacatcaaagtatgtgttggccaccaccaactgtcccacaccagtcatgcccgatcacccgagggctatggcgttctggcgccgcataaccttcattgacgtcactgcccccacaattgaacaatggcttgttgacaaccccgggcgcaaggcaccgcctagccttttcaaagatgacttttcccacttgcagtgttccgtccgcgggtacactgcctatgatgaaaaaggcaacacgctcagtggcaaggtggctcgggccaggtacgtgtcagtcaataacctgctcgacctgatcaaagagaaatacaacagtgaagccactgacatcaaacacctgtggttcaccgttccgcagggcattcacaagcaagcacgagatatcatactggggtggctgcggttccactcctaccccaacaccgtggctgacaacatacccctttcagaagtcagggatcccacctgcttcgggtatgtagtcatttctgatgtggacccacctcggcacgtgagtgaccatgtggcacacattgaagtggagtcaatattgcgcaccgacatcatggaccttttgagagagggaggcggaggcctgttcagggcgttgaaggttaaaagtgccccccgaaattgtgtgatcaacaaagtcatgatgcaagcgcaccacaccaccctgcaggtgctcaccagccaggaaccaaacccaccaaacctgccccgacctagacgcttggtttttgttgagtcaccaattgacatcattagcgccctgcgacaccacgtggggttctgcaccattccaggcatagtcaagttgatcacatcaggagtgggcctgggagtggaaaacctgggcaatttcttgcagtccattgccggcaacgtcagattccctttgcaatccgagtgctcactgctcaggacacctggcggtgacgtgctattttacacctcagggcaagcagcagtgtgggcgacacccgcacgttttccaatcgtcactccgggagaggcatctgttggcaaagatgtgtgttctgagtcctcctggtgggacatcctcaaggccctgtttagcacgctggtggtggccttcggccccatagccacactggtgctgaccgcacacaacctggcctacctgaacacccgtgaaaacaccttgagtgaggcaaaaggtaaaaacaagcgtggccgcggtgcccgcaaaaccatcgccctcagggacgatgagtatgatgaatggcaggacatcattcgtgactggcgcaaggaaatgactgtccaacagtttttagatctgaaagaacgcgccctttccggcgcctctgatcctgattcgcagaggtacaatgcctggctggaactgcgcgcaaagagattgagtgccggggcctaccagcatgcagttgtggacatcattgggaagtcaggacataggcgtgaggtgattcgcacgcagatgatgcgggcgccccgtgaaccaaaagcggacacctatgatagtgaggggcgtggttacgtggtgcccatgactgcccaggaaaaacacacaggctgggcagtgcacattggaaatggccgactggtcacttgcacacatgtggccaacatgtgcgatcgcgtggctgagatggagttcaagatcacagaaacggatcgcgacacttgtattatcaccgccccactcggccacctacccagtgttgcccttggggacggaccgccagccttttacaccactaatttccaccccatccgtgtgttggatgagggcagttgggacaccaccaccacccgtgtcaccggttggcgtgttgtcattaacaacggcaccacaactgcccctggtgattgcggccaaccgtacttaaacacacgccggcagctcgttggcgtgcatgctgcaacaagcacatgtggggtcaagaaactggtatccagggtgcagaccaaaagggcatcaaaggccacgttcccttggaagggcctgcctgtcgccaacatgcccgatgctgggggcctgcccactggcacgcggtaccaccgctctgtcgcgtggcccaagcagttgcctgaggagacgcatgcccctgccccctatggtgtcaatgatccccggtaccccttctctcaacaccagatgattgccaacaacctacaaccctacaccaagaccccagtcgcattagaccagaccttgctacaacgtgctgtcaaacacacgaagggttacctggaccaagtcatcggaacacacagatccccaaacctcacctacgcggcagcagtggactcgatggcccacgacacagcgtgtgggccaaatttatctggccgcaagaaggactacatgacagatcagggggaacccattggaccactcaaacaaatgttagaggaggcttgggacatggctcaccggggtgttccgcgcaaacacgagtacaagctcgcgctcaaggatgaactccggcccatagaaaagaatgatcaaggcaagaggcgcctgctttggggctgtgacgctggggtgtcgctggtggccaacgccgtgtttaaacctgtagccgagcgcttggtggacaccgtgcccatgcaccctgtagctgttggcatatgtatggacagcccccaaattgagcagatgaaccaggccctaactggtagggtgctgtactgcttggactacagcaagtgggactcaacgcagaacccagctgtgacaagcgccagtgttgacatcctggcctcgtacgctgaggacacaccactcacatcagctgccatagccacactgtgttcgccagccatcggtcgacttgacgacattggtttgaccgtcacaacgggtctcccctctggcatgccgttcaccagcgtcataaattcagtcaaccacatggtgtattttgccatggctgtgcttgaggcttatgaggagttcaaggtgccctacatggggaacatttttgacaatgagactgtttacacctacggggatgactgtgtgtatggcctgacaccggctacagcttccatcatgccggtggtggtcaaaaacctgaccagttacggactggttccaactgctgcagacaaatcccaaaccattgaacccactgacaccccagttttcctcaaacgcaccttttcccagacaccctttggactgcgggcactgcttgatgaaactagtctggcgcgacagtgctactgggttaaagctaaccgcacaacagacctttttgagccagcagctgttgatgtggagattaggaaaaaccagttggaggtcatgctcgcgtacgccagccagcatccaagggcagtgtttgacaagcttgcaaacatggttgaggcaactgcaaatgctgagggttatcaagttgtgaatgttgattgggccaacgcagtggcaacgtacaacgcatggtacggtggcactgacggtggccacgctcccaccaatgaagatgaggagccagaagtgttcgtgatggaggcgcctgccccaacccgtccggttgcgagcaacccagaaggcactgagaccagtgatgagtccaggccagttcagccgcctgggcctgtgcctgtggccgcagcacaggctctggagatggccgttgccacagggcaaatcaatgacaccatccccagtgtggtcaggcaaacattcagtacttacaccaatgttacctggaccacgcgtcaacccgcaggaaccctgcttgcccgaatgtccctagggccaggattgaacccttacacgcttcatttgtccgccatgtgggccggttggggtgggtcgtttgatattaaggtggtgatatcagggtctggcatatatgcaggcaaactgctgtgtgcactcataccacctggggttgaccctagcactgtggatcaacccggggcctttccccacgcgcttgtggatgcgcgcaccactgatggtgtttcatttaacttgggggatgtcagggcagtggattaccatgaaacaggtgctggtggaaacgtggcatgcttggcactttacgtgtaccagccgctcatcaacccctttgaaaccaccacatctgctgccatggtgacagtcgagacccgtcctggcgcagactttgggtttaccctactcaagcctccaaaccagaccatggaggtgggacttgaccccaggtcgctcctgccccgcactgcaagaacactgcgggggaacaggtttggcaggcccatcagatctgtgattatagtaggtttggcacaacaaatcaacaggcatttctccgcagaaggcaccacacttggttggtccacggccccaattggcccctgtgcaggccgcatcaacaccaagtacactggtagtgcgggcaaggtggtggctcaactgttacctttgagcaatgggcccctttacccaaacatcatcaaccactacccagacgtggctgcatcaacaactttcagtggagggtctagcataacccctgacatgacgtgtgggggaggacccatggtgctcttcaacgatgtgggtgatgtggtggagaccgtctcctaccaaatgaggttcatagcctcacaggccacatctcaaagtaccacactcaccgacaagatcaatgcaacatcaatgtcagtggtcagttttgacagctcccggaatgacttcccccaatcaaatgacaatgtgggtattgagttaacctacacttgtggcaacacaccgatcaatgggaacgtcacccagttcatggaccgccaatacacctttggcgcacaggggcccaacaacatcatgctttgggtggagtctgtgcttggcacgcacactggcaacaacagagtctacagctcacaaccagataccgtgtctgctgcactgcagggccagccctacaacataccacaggggtacatggccgtgtggaatgtcaacgcggacagtgctgactttcaaatagggctgaggcgcgatggctactttatcaccaatggagccattggcacagaaatggctatttcagatgacaccaccttcaccttcaatggaatgtacaccctaaccactcccctcattggaccaagtgggacaactggaaggtccgttcacagctcacgatga

SEQ ID NO: 27 - RNA, 6765 nt, Sapporo virus
  source 1..6765: mol_type = genomic RNA
atggctaactgccgtccgttgcctattgggcacttgcccaacaggatttttagcaccccacgactcaccccagggtgggtgtgggcgtgcaccaaggaagccatgtttaagctagagtggcttcaagatccggtggtcattcgacagccggatgtttacatcgcgcaaggggtggtggatgacttcttccgccccaagcgtgtcttacagggtgatccacagctcatagcacaggtgctgcttggtgatgccagcggcccgctggtcgggcctgtttcaatgcagcaactaacatcgttgctacatgaggtttcacaggcactaaatgaccacaagcatcccctggccaacagatacaccaaagcctctttgcaaagatatgcagacaccttgtccaactacatacctctagtggagatattaactggaccaaaggacctcaccccgcgtgacgtgctcgaacaactcgcggctgggcgtgaatgggagtgtgtcccagacagcgccctcaagaaagtttttcgggacatgtggcaatacatttgtgagggctgtgacagcgtgtacatcaaattgcaggatgtgaagcgcaagataccccacattgacaccaccgtgttgaaacagttctttattacactcacagacaccatatcaatggcaacagcgctagacaccaaaacctggttggcacacatactgggttggctcaagcccacctgtttggtgatgatcatgcaacaacacatcaactctccccaggggtgggcggccacactaactgcccttgcagagctctactatggcatcatgccgctcaccgaggtgctcgggtccattgccggttgggtcactgacaagtttgctgatgtgaccaccaacacctggggcaagttcaaagcttggtgggacaatctttacacaccgcaagccggcaatgacttgatcatactaggtggtgttgtcgggctggtgtacttcatggtttttggagatgcacccacccaaatgttcaccaagaagctgatgaaggtgtgtgggttcatcacatccactgtggcagcgatcaaggctgccatgtggatagttgattattttaagcaacgcgagcatgaacatcaggttcgcgtcaccttggccaggtgggccgcactccaggaggttatcaagcagaataggtgtgctggccttagtgaaatcaccaaactgaaggagtgctgtgaagtcttgctgaatgaggtcaccgaattgatgtacaagctgggtgcctcgcctctagcggggttgattcgtagcacctctgacgtcatacaaaccaccatcaatgatttggctcagctcatggcgtacgacacccaacgcaagccccccgccatgatcgtgtttggtgggccacctggcataggtaagacccgattggttgaagctctggccagacagcttggggaggtcagccacttcaccatgaccgtcgaccactatgacacctacacaggcaatgcagtggcaatatgggacgaatttgatgtggactcaaagcaggcattcatcgaggccaccatcggtattgtcaattgtgccccctaccctttaaactgcgaccggccagaggcaaagggcagggtgttcacgtcaaagtatgtgttggccaccaccaactgtcccacaccagtcatgcctgaccacccgagggccatggcgttctggcgtcgcataaccttcattgatgtcactgcccccacaattgaacaatggttggttgacaaccccgggcgcaaggcaccggctagcctttttaaagaggacttctcccacctgcagtgctccgtacgtgggtacacagcctatgacgaaaaaggcaacacgctcagtggcaaagtggctcgggctaggtacgtatcagtcaacaacctgctcgacctgatcaaagagaagtacaacagtgaagccactgacattaaacatctgtggttcaccgttccacagagcatccacaagcaggcacgagacatcatactggggtggctgcggttccactcctaccccaataccgtggctgacaacatacccctttcggaagtcagggaccccacctgcttcgggtatgtggtcatctctgacgtggacccgccccggcatgtgagtgaccatgtggcacacattgaagtggagtcaatactgcgcaccgacatcatggaccttttgagagagggaggcggaggcctgttcagggcgttgaaggtgaaaagcgcccctcggaattgtgtgatcaacaaagtcatgatgcaagcgcaccacaccaccctgcaggtgcttaccaaccaggaaccaaacccaccaaacctgccccgacctagacgtttggtttttgttgagtcaccaattgacatcatcagcgccctgcgacaccacgttgggttctgcaccattccaggcatagtcaagttgatcacctcgggagtgggcctgggtgtggaaaacctgggcaacttcttgcaatccatcgccggcaacgtcagattccctttgcagtccgagtgctcactactcaggacacccggcggtgatgtgctattctacacctcagggcaagcagcagtgtgggcgacacccgcacgttttccaatcatcactccgggagaggcgtctgttggcaaagatgtgtgttctgagtcctcctggtgggacatcctcaaggccttgtttagcacgctggtggtggccttcggccccatagccacattggtgctgactgcacacaacttggcctacttgaacacccgtgagaacaccttgagtgaggcaaaaggtaaaaacaagcgaggtcgcggtgcccgcaaagccatcgccctcagggacgatgagtatgatgagtggcaggacatcattcgtgactggcgcaaggaaatgactgtccaacagtttttagacctgaaagaacgcgccctttccggtgcctctgaccccgattcgcagaggtataatgcctggttggaactgcgcgcaaaaaggttgagtgccggggcctaccagcatgcagttgtagacatcattgggaaatcaggacacaggcgcgaggtgattcgcacacaggtgatgcgggcgccccgtgaaccaaaggcagacacctacgatagtgaggggcgtggttacgtggtgcccatgactgcccaggaaaaacacacaggctgggcagtgcacattggaaatggccggctggtcacttgcacacatgtggccaacatgtgcgatcgcgtggctgaggtggagttcaagatcacagaaacagatcgcgacacttgcatcatcactgccccactcggccacctacccagtgttgcccttggggatggaccaccggccttttacaccaccaactttcaccccatccgcgtgttggatgagggcagttgggataccaccaccacccgtgtcaccggttggcgtgttgtcattaacaatggcaccacaacagcccctggtgattgcggccaaccgtacttgaacgcacgccggcagctcgttggtgtgcatgcagcaacaagcacatgtggggttaagaaactggtgtctagggtgcagaccaaaagggcatcaaaggccacattcccatggaagggcctgcctgtcaccaacatgcccgatgccgggggcttgcccactggcacgcggtaccaccgctccatcgcgtggcccaagcagttgcctgaggagacacatgcccctgccccctatggcgtcaatgatccccggtaccccttctcccaacaccaaatgatcgccaacaacctacagccctacaccaaaaccccagtcgcactagaccagaccttgctgcaacgtgctgttaaacatacaaaggggtatctagaccaagtcattggaacacacagatccccaaacctcacctacgcggcagcagtggactcgatggcccatgacacagcatgtgggccaaatttgcctggccgcaagaaggattacatgacagatcagggggaacccattggaccacttaaacaaatgttggaggaagcttgggacatggctcaccgaggtgtcccgcgcaagcacgagtacaagcttgcgctcaaggatgaactccggcccatagaaaagaatgatcaaggcaagaggcgcctcctttggggctgtgacgctggggtgtcgctggtggccaacgccgtgtttaaacctgtggccgagcgcctggtggataccgtacccatgcacccggtggctgttggcatatgcatggacagcccccagattgaacagatgaaccaggccttaactggtagggtgctgtactgcctggactacagcaagtgggactccacgcaaaatccagctgtgacaagcgccagtgttgacatcttggcctcatacgctgaggacacaccactcacatcagctgccatagctacactgtgttcgccagccatcggtcgacttgacgacattggcttgactgttacaacgggtctcccctctggcatgccattcaccagtgtcataaattcagtcaaccacatggtgtattttgccatggctgtgcttgaggcttacgaggagttcaaggtgccctacatggggaacatttttgacaatgagactgtttacacttacggggatgactgtgtgtatggcctcacaccggcaacagcttccatcatgccggtggtggtcaaaaacctgaccagttacgggctggttccaactgctgcagacaaatcccaaaccattgaacccactgacaccccagttttcctcaaacgcaccttttcccaaacaccctttggactgcgggcactgcttgatgaaactagtctggcgcgacagtgctactgggttaaagctaaccgcacaacagacctttttgagccagcagctgttgacgtggaaatcaggaaaaaccagttggaggtcatgctcgcgtacgccagtcagcatccaaggatagtgtttgacaagcttgcaagtatggttgaggcaactgcaaatgccgagggctaccaagttgtgaatgttgattgggccaacgcagtggcaacgtacaacgcatggtacggtggcactgacggtggccacgctcccaccaatgaagatgaagagccagaagtgttcgtgatggaggcgcctgccccaacccgttcggtggcgagcaacccagagggtacccaaaccagcaatgaatccaggccagttcagccagccgggcccatgcccgtggccactgcccaggcccttgagatggctgttgccactggacaagtcaatgacacaatccccagtgtggtcagagaaactttcagcacctacaccaatgtcacctggactacacgtcagccagcgggaaccttgctcgcccggatgaccctagggccaggcctgaacccctacacactccacctgtctgccatgtgggctggctggggagggtcatttgaaattaaggtggtgatatcggggtctggcttgtatgcaggcaaattgctgtgcgcgctcataccacctggggtggaccccagtgctgtggaccagcccggggcttttccccatgcacttgtggatgcgcgcatcactgagggcgtcaccttcacccttggggatgtcagagcagtggattaccacgaaacaggagctggtgggaccatcgcttgcttggcactttatgtgtaccaaccactcatcaacccctttgaaactgctttgtcagccgccatggtgaccatcgagacccgccctggcccagactttgggttcaccctgcttaagcctcctaaccaaaccatggaggcaggacttgaccccagatcgcttctgccccgcacagcaaggacactgcggggaaacaggtttggcaggcccatcacaggcgtggtcatagtcggcatggcacaccagatcaacaggcacttctcagccgcgggcaccacgcttggctggtccacagccccaattggtccttgtgtaggacgtattaattctaggtacaccaacaacggcggccttgccgtgctctcgttgcaacccctaagcaatgggcccctctaccctaacatcatcaaccactacccagatgtggctgcttcaaaagctttcaacacaagcactaccctgagtgatagcaccacgtgtggtgggggacctatggtgattttcaatgatgtgggtgatgtggttgagaccgtgtcctaccaaatgagattcatagcctcacaagccacctcccaaacacccacactcgttgattacatcaatgcaacatcaatgggattggtcagttttggcaattctcggggggactttggctcaggccagctcaatgtgggcgtcgagttgacctacacctgtggcaacacagcgatcaatgagaaagtcactacgttcatggatcgccaatacacatttggtgcacagggtcccaacaacatcatgctctgggtggagactgtactcggcacgcacacgggcaacaacactgtgtacagctcgcaacccgacactgtgtctgctgcactgcagggtcagccatacaacataccagatggatacatggctgtgtggaatgttaatgcaaacagtgccgatttccagataggcttgaggcgtgatggcttctttgtcaccagtggggccattggcacgcgcatgaccatctcagaggacaccaccttcacctacgccggcatgttcaccctcaccacccccctcattggaccaagtgggatgacaggacggtcccttcacagctcaccatga

SEQ ID NO: 28 - RNA, 6765 nt, Sapporo virus
  source 1..6765: mol_type = genomic RNA
atggctaactgccgtccgttgcctattgggcacttgcccaacaggatttttggcaccccacgactcaccccagggtgggtgtgggcgtgcactaaggaagccatgtttaagctagattggcttcaagatccggtggtcatccgacagccggatgtttacatcgcgcaaggggtggtggatgatttcttccgccccaagcgtgttttacagggtgatccacagctcatagcacaggtgttacttggtgatgccagcggcccactggtcgggcctgtctcaatgcagcaactaacatcgttgctgcatgaggtctcacaggcactgaatgaccacaagcaccccctggccaacagatacaccaaagcctctttgcaaagatatgcagacaccttgtccaactacatacctctagtggagatattaactggaccaaaggacctcaccccgcgtgacgtgctcgagcaactcgcggctgggcgtgaatgggagtgtgtcccagacagtgccctcaagaaggtctttcgggacatgtggcaatacatttgtgagggctgtgacagcgtgtacatcaaactacaggaagtgaagcgcaagataccccacattgacaccaccgtattgaaacagttctttatcacacttacagacaccatatcaatggcaacagcgctggacaccaagacctggttggcacacatactgggttggctcaagcccacctgtttggtgatgattatgcaacaacacatcaactccccccaggggtgggcagccacactaactgcccttgcagagctttactacggcatcatgccgctcaccgaggtgctcgggtccatcgctggttgggtcactgacaagtttgctgacgtgaccaccaacacctggggcaagttcaaggcttggtgggacaatctttacacaccgcaggctggcaatgacttgatcatactaggtggtgttgtcgggctggtgtacttcatggtttttggagatgcacccacccagatgtttaccaagaagctaatgaaggtgtgtgggttcatcacatctactgtggcagcgatcaaggctgccatgtggatagttgattactttaagcaacgcgagcacgaacaccaggttcgcgttaccttggccaggtgggccgcactccaggaggtcatcaagcagaacaggtgtgctggccttagtgaaatcgccaaactgaaggagtgctgtgaagtcttgctgaatgaggtcaccgaattgatgtataagctgggcgcctcgcctctagcggggctgattcgtagcacctctgatgtcatacagaccaccatcaatgatttagcccagctgatggcgtacgacacccaacgcaagccccccgccatgatcgtgtttggtgggccacctggcataggcaaaacccggttggttgaagctctggccagacagcttggggaggtcagccatttcaccatgaccgtcgaccactacgacacctacacaggcaacacagtggcaatatgggacgaatttgatgtggactcaaagcaggcatttatcgaggccactatcggtattgtcaattgtgccccctaccccttaaactgcgaccggccagaggcaaagggcagggtgttcacgtcaaagtatgtgttggccaccaccaactgtcccacaccagtcatgcctgaccacccgagggccatggcgttctggcgtcgcataaccttcatcgatgtcactgcccccacaattgaacaatggctgattgacaaccccgggcgcaaggcaccagccagccttttcaaggaggacttctcccacctgcagtgctccgtgcgtggttacacagcctatgacgaaaaaggcaacacgctcagtggcaaggtggctcgagctaggtatgtgtcagtcaataacctgctcgacctcatcaaagagaagtacaacagtgaagccactgacatcaaacacctgtggttcaccgtcccacaaagcatccacaagcaggcacgagacatcatactggggtggctgcggttccactcctaccccaacaccgtggctgacaacatacccctttcagaagtcagggaccccacctgcttcgggtatgtggtcatttctgacgtggacccgcctcgacatgtgagtgaccacgtggcacacattgaagtggagtcaatattgcgcaccgacatcatggaccttttgagagaggggggcggaggtctgttcagagcattgaaggtcaaaagcgcccctcggaactgtgtgattaataaagtcatgatgcaagcgcaccacaccaccctgcaagtgcttactagccaggaaccaaacccaccaaacctgccccgacccagacgtttagtttttgttgagtcaccaattgacatcattagcgccctgcgacatcacgtggggttctgcaccattccaggcatagtcaagttgattacatcgggagtgggcctgggcgtggaaaacctgggcaatttcttgcaatccatcgccggcaatgtcagatttcctctgcagtccgagtgctcactactcaggacacccggcggtgatgtgctattttacacttcagggcaagcggcagtgtgggcgacacccgcacgttttccaatcatcactccgggagaggcgtctgttggcaaagatgtgtgttctgagtcctcctggtgggacatcctcaaggccttgttcagcacgctggtagtggccttcggccccatagcaacattggtgctaactgcacacaacttggcctacttgaacacccgtgagaacaccttgagtgaggcaaaaggtaaaaacaagcgtggtcgcggtgcccgcaaagccatcgccctcagggacgatgagtatgatgaatggcaggatatcattcgtgactggcgcaaggaaatgactgtccaacagttcttggacctgaaagagcgtgccctttccggtgcctctgaccctgactcgcagaggtacaatgcctggctggaactccgcgcaaagaggttgagtgccggggcctaccagcatgcagttgtggacatcattgggaaatcaggacataggcgcgaggtgattcgcacacaggtgatgcgggcgccccgtgagccaaaagcagacacctatgacagtgaggggcgtggttacgtggtgcccatgactgcccaggaaaaacacacaggctgggcagtgcatattggaaatggccgactagtcacttgcacacatgtggccaacatgtgcgatcacgtagctgaggtggagttcaagatcacagaaacagatcgcgatacttgcatcatcactgccccgctcggccacctacccagcgttgcccttggggatggaccaccggccttttacaccaccaacttccaccccatccgtgtgttggacgagggtagctgggacaccaccaccacccgtgttaccggttggcgtgttgtcatcaacaacggcaccactactgcccctggtgattgcggtcaaccgtacttgaacgcacgccggcagctcgttggtgtgcatgcagcaacaagtacatgtggggtcaagaaactggtgtctagggtgcagaccaaaagggcatcaaaggccacgtttccttggaagggcctgcctgtcaccaacatgcccgatgccgggggcttgcccactggcacgcggtaccaccgctctatcgcgtggcccaagcagttgcctgaggagacacatgcccctgccccctatggtgtcaatgatccccggtattccttctcccaacatcaaatgatcgccaacaacctgcagccctacaccaaaaccccagtcgcattagatcaaaccttgctgcaacgtgctgtcaaacacacaaagggctacctagatcaggtcattggaacacacagatccccaaacctcacctacgcagcagcagtggactcgatggcccacgacacagcatgtgggccaaacttgcctggccgcaagaaggattacatgacagatcagggggagcccattggaccacttaaacaaatgttggaggaagcttgggacatggcccaccgaggtgtcccgcgcaaacatgagtacaagcttgcactcaaggatgaactccggcccatagaaaagaatgaccaaggcaagaggcgcctcctttggggctgcgacgctggggtgtcgctggtggccaacgccgtgttcaaacctgtggctgagcgcttggtggacactgtgcccatgcacccagtggctgttggcatatgtatggatagcccccaaattgagcagatgaaccaggccttgactggtagggtgctgtactgcctggactacagcaagtgggactccacgcaaaatccagccgtgacaagcgccagtgttgacatcttagcctcgtacgctgaggacacaccactcacgtcagctgccatagccacactgtgttcgccagccatcggtcgacttgacgacattggtttgactgttacaacgggtctcccctctggcatgccgttcaccagtgtcataaattcaatcaaccacatggtgtattttgccatggctgtgcttgaggcttacgaggagttcaaggtgccctacatggggaacatttttgataatgagactgtctacacctatggggatgactgtgtatatggcctgacaccggcaacagcctccatcatgccggtggtggtcaaaaatctgaccagttacggactggttccaactgctgcagacaaatcccaagctattgaacccactgacaccccaattttcctcaaacgcaccttttcccaaacaccctttgggttgcgggcactgcttgatgaaactagtctggcgcgacagtgctactgggtcaaggctaaccgcacaacagacctttttgagccagcagctgttgatgtggaaattaggaaaaaccaattggaggtcatgctcgcgtacgccagtcagcatccaagggcagtgtttgacaagcttgcaggcatggttgaggcaactgcaaatgccgagggttaccaagttgtgaatgttgattgggccaacgcagtggcaacgtacaacgcatggtacggtggcactaacggtggccacgctcccaccaatgaagatgaagagccagaagtgttcgtgatggaggcgcctgccccaacccgttcggtggcgagcaacccagagggtactcaaaccagcaatgaatccaggccagtccagccagccgggcccatgcccgtggccactgcccaggcccttgagatggcagttgccactggacaagtcaatgacacaatccccagcgtggtcagagaaactttcagcacctataccaatgttacctggaccacgcgccagccagcagggaccctgcttgctcggatgaccctagggccaggcctgaatccctacacactccacctgtccgccatgtgggctggctggggagggtcatttgaaatcaaggtggtgatatcggggtctggcttgtatgcaggcaagttgctgtgcgcactcataccacctggcgtagaccctagtgctgtggatcagcccggggcttttccccacgcacttgtggatgcgcgcatcactgagggtgtcaccttcacccttggggacgttagggcagtggattaccacgaaacaggagctggtgggaccatcgcttgcctggcactttatgtgtaccaaccactcatcaacccctttgaaactgctttgtcagccgccatggtaacaatcgaaacccgccctggtccagactttgggttcaccctgcttaagccccctaaccaaaccatggaggcaggacttgaccctagatcactcctgccccgcacagcaaggacactgcggggaaacaggtttggcaggcccattacaggcgtggttatagtcggcatggcataccaaatcaacaggcatttctcagctgcgggcaccacgcttggctggtccacggccccaattggtccttgtgtgggacgcatcaattctaggtacacaaacaacggcggccttgccgtgctctcgatgcaacccctgagcaatgggcccctttaccccaacatcatcaaccattacccggatgtggctgcttcaaaagcattcaacacaagcactaccctgagtgatagcaccacgtgtggtgggggacccatggtgcttttcaatgatgtaggtgatgtggttgagaacgtgtcctaccaaatgagattcatagcctcacaagccacctcccaaacacccacactcgttgattacatcaatgcaacatcaatgggattggtcagttttggcaattctcggggggactttggctcaggccagctcaatgtgggcgttgagttgacctacacctgtggtaacacagcgatcaatgagaaagtcactacgttcatggatcgccaatacacatttggtgcacaggggcccaataacatcatgctctgggtggagactgtgctcggcacgcacacgggtaacaacactgtgtacagctcgcaacccgacactgtgtctgccgcactgcagggccagccctacaacataccagatggttacatggctgtgtggaatgtcaatgcaaacagtgccgatttccagataggcttgaggcgtgatggcttctttgtcaccagtggggccattggcacgcgcatgaccatctcagatgacaccaccttcacctacgctggcatgttcgccctcaccacccctctcattggaccaagtgggatgacaggacggtcccttcacagctcaccatga

SEQ ID NO: 29 - protein, 2254 aa, Sapporo virus
MANCRPLPIGRLPNRIFSTPRLTPGWVWACTKKAMFKPEWLQDPVVIRQPDVYTAQGVVDDFFRPKRVLQGDPQLIAQVLLGDASGPLVGPVSMQQLTSLLHEVSQALNDHKHPLANRYTKASLQRYADTLSNYIPLVEILTGPKDLTPRDVLEQLAAGREWECVPDSALKKVFRDMWQYICEGCDSVYIKLQDVKRKIPHIDTTVLKQFFITLTDTISMATALDTKTWLAHILGWLKPTCLVMIMQQHINSPQGWAATLTALAELYYGIMPLTEVLGSIAGWVTDKFADVTTNTWGKFKAWWDNLYTPQAGNDLIILGGVVGLVYFMVFGDAPTQMFTKKLMKVCGFITSTVAAIKAAMWIVDYFKQREHEHQVRVTLARWAALQEVIKQNRCAGLSEVTKLKECCEVLLNEVTELMYKLGASPLAGLIRSTSDVIQTNINDLAQLMAYDTQRKPPAMIVFGGPPGIGKTRLVEALARQLGEVSHFTMTVDHYDTYTGNAVAIWDEFDVDSKQAFIEATIGIVNCAPYPLNCDRPEAKGRVFTSKYVLATTNCPTPVMPDHPRAMAFWRRITFIDVTAPTIEQWLVDNPGRKAPPSLFKDDFSHLQCSVRGYTAYDEKGNTLSGKVARARYVSVNNLLDLIKEKYNSEATDIKHLWFTVPQGIHKQARDIILGWLRFHSYPNTVADNIPLSEVRDPTCFGYVVISDVDPPRHVSDHVAHIEVESILRTDIMDLLREGGGGLFRALKVKSAPRNCVINKVMMQAHHTTLQVLTSQEPNPPNLPRPRRLVFVESPIDIISALRHHVGFCTIPGIVKLITSGVGLGVENLGNFLQSIAGNVRFPLQSECSLLRTPGGDVLFYTSGQAAVWATPARFPIVTPGEASVGKDVCSESSWWDILKALFSTLVVAFGPIATLVLTAHNLAYLNTRENTLSEAKGKNKRGRGARKTIALRDDEYDEWQDIIRDWRKEMTVQQFLDLKERALSGASDPDSQRYNAWLELRAKRLSAGAYQHAVVDIIGKSGHRREVIRTQMMRAPREPKADTYDSEGRGYVVPMTAQEKHTGWAVHIGNGRLVTCTHVANMCDRVAEMEFKITETDRDTCIITAPLGHLPSVALGDGPPAFYTTNFHPIRVLDEGSWDTTTTRVTGWRVVINNGTTTAPGDCGQPYLNTRRQLVGVHAATSTCGVKKLVSRVQTKRASKATFPWKGLPVANMPDAGGLPTGTRYHRSVAWPKQLPEETHAPAPYGVNDPRYPFSQHQMIANNLQPYTKTPVALDQTLLQRAVKHTKGYLDQVIGTHRSPNLTYAAAVDSMAHDTACGPNLSGRKKDYMTDQGEPIGPLKQMLEEAWDMAHRGVPRKHEYKLALKDELRPIEKNDQGKRRLLWGCDAGVSLVANAVFKPVAERLVDTVPMHPVAVGICMDSPQIEQMNQALTGRVLYCLDYSKWDSTQNPAVTSASVDILASYAEDTPLTSAAIATLCSPAIGRLDDIGLTVTTGLPSGMPFTSVINSVNHMVYFAMAVLEAYEEFKVPYMGNIFDNETVYTYGDDCVYGLTPATASIMPVVVKNLTSYGLVPTAADKSQTIEPTDTPVFLKRTFSQTPFGLRALLDETSLARQCYWVKANRTTDLFEPAAVDVEIRKNQLEVMLAYASQHPRAVFDKLANMVEATANAEGYQVVNVDWANAVATYNAWYGGTDGGHAPTNEDEEPEVFVMEAPAPTRPVASNPEGTETSDESRPVQPPGPVPVAAAQALEMAVATGQINDTIPSVVRQTFSTYTNVTWTTRQPAGTLLARMSLGPGLNPYTLHLSAMWAGWGGSFDIKVVISGSGIYAGKLLCALIPPGVDPSTVDQPGAFPHALVDARTTDGVSFNLGDVRAVDYHETGAGGNVACLALYVYQPLINPFETTTSAAMVTVETRPGADFGFTLLKPPNQTMEVGLDPRSLLPRTARTLRGNRFGRPIRSVIIVGLAQQINRHFSAEGTTLGWSTAPIGPCAGRINTKYTGSAGKVVAQLLPLSNGPLYPNIINHYPDVAASTTFSGGSSITPDMTCGGGPMVLFNDVGDVVETVSYQMRFIASQATSQSTTLTDKINATSMSVVSFDSSRNDFPQSNDNVGIELTYTCGNTPINGNVTQFMDRQYTFGAQGPNNIMLWVESVLGTHTGNNRVYSSQPDTVSAALQGQPYNIPQGYMAVWNVNADSADFQIGLRRDGYFITNGAIGTEMAISDDTTFTFNGMYTLTTPLIGPSGTTGRSVHSSR

SEQ ID NO: 30 - protein, 2254 aa, Sapporo virus
MANCRPLPIGHLPNRIFSTPRLTPGWVWACTKEAMFKLEWLQDPVVIRQPDVYIAQGVVDDFFRPKRVLQGDPQLIAQVLLGDASGPLVGPVSMQQLTSLLHEVSQALNDHKHPLANRYTKASLQRYADTLSNYIPLVEILTGPKDLTPRDVLEQLAAGREWECVPDSALKKVFRDMWQYICEGCDSVYIKLQDVKRKIPHIDTTVLKQFFITLTDTISMATALDTKTWLAHILGWLKPTCLVMIMQQHINSPQGWAATLTALAELYYGIMPLTEVLGSIAGWVTDKFADVTTNTWGKFKAWWDNLYTPQAGNDLIILGGVVGLVYFMVFGDAPTQMFTKKLMKVCGFITSTVAAIKAAMWIVDYFKQREHEHQVRVTLARWAALQEVIKQNRCAGLSEITKLKECCEVLLNEVTELMYKLGASPLAGLIRSTSDVIQTTINDLAQLMAYDTQRKPPAMIVFGGPPGIGKTRLVEALARQLGEVSHFTMTVDHYDTYTGNAVAIWDEFDVDSKQAFIEATIGIVNCAPYPLNCDRPEAKGRVFTSKYVLATTNCPTPVMPDHPRAMAFWRRITFIDVTAPTIEQWLVDNPGRKAPASLFKEDFSHLQCSVRGYTAYDEKGNTLSGKVARARYVSVNNLLDLIKEKYNSEATDIKHLWFTVPQSIHKQARDIILGWLRFHSYPNTVADNIPLSEVRDPTCFGYVVISDVDPPRHVSDHVAHIEVESILRTDIMDLLREGGGGLFRALKVKSAPRNCVINKVMMQAHHTTLQVLTNQEPNPPNLPRPRRLVFVESPIDIISALRHHVGFCTIPGIVKLITSGVGLGVENLGNFLQSIAGNVRFPLQSECSLLRTPGGDVLFYTSGQAAVWATPARFPIITPGEASVGKDVCSESSWWDILKALFSTLVVAFGPIATLVLTAHNLAYLNTRENTLSEAKGKNKRGRGARKAIALRDDEYDEWQDIIRDWRKEMTVQQFLDLKERALSGASDPDSQRYNAWLELRAKRLSAGAYQHAVVDIIGKSGHRREVIRTQVMRAPREPKADTYDSEGRGYVVPMTAQEKHTGWAVHIGNGRLVTCTHVANMCDRVAEVEFKITETDRDTCIITAPLGHLPSVALGDGPPAFYTTNFHPIRVLDEGSWDTTTTRVTGWRVVINNGTTTAPGDCGQPYLNARRQLVGVHAATSTCGVKKLVSRVQTKRASKATFPWKGLPVTNMPDAGGLPTGTRYHRSIAWPKQLPEETHAPAPYGVNDPRYPFSQHQMIANNLQPYTKTPVALDQTLLQRAVKHTKGYLDQVIGTHRSPNLTYAAAVDSMAHDTACGPNLPGRKKDYMTDQGEPIGPLKQMLEEAWDMAHRGVPRKHEYKLALKDELRPIEKNDQGKRRLLWGCDAGVSLVANAVFKPVAERLVDTVPMHPVAVGICMDSPQIEQMNQALTGRVLYCLDYSKWDSTQNPAVTSASVDILASYAEDTPLTSAAIATLCSPAIGRLDDIGLTVTTGLPSGMPFTSVINSVNHMVYFAMAVLEAYEEFKVPYMGNIFDNETVYTYGDDCVYGLTPATASIMPVVVKNLTSYGLVPTAADKSQTIEPTDTPVFLKRTFSQTPFGLRALLDETSLARQCYWVKANRTTDLFEPAAVDVEIRKNQLEVMLAYASQHPRIVFDKLASMVEATANAEGYQVVNVDWANAVATYNAWYGGTDGGHAPTNEDEEPEVFVMEAPAPTRSVASNPEGTQTSNESRPVQPAGPMPVATAQALEMAVATGQVNDTIPSVVRETFSTYTNVTWTTRQPAGTLLARMTLGPGLNPYTLHLSAMWAGWGGSFEIKVVISGSGLYAGKLLCALIPPGVDPSAVDQPGAFPHALVDARITEGVTFTLGDVRAVDYHETGAGGTIACLALYVYQPLINPFETALSAAMVTIETRPGPDFGFTLLKPPNQTMEAGLDPRSLLPRTARTLRGNRFGRPITGVVIVGMAHQINRHFSAAGTTLGWSTAPIGPCVGRINSRYTNNGGLAVLSLQPLSNGPLYPNIINHYPDVAASKAFNTSTTLSDSTTCGGGPMVIFNDVGDVVETVSYQMRFIASQATSQTPTLVDYINATSMGLVSFGNSRGDFGSGQLNVGVELTYTCGNTAINEKVTTFMDRQYTFGAQGPNNIMLWVETVLGTHTGNNTVYSSQPDTVSAALQGQPYNIPDGYMAVWNVNANSADFQIGLRRDGFFVTSGAIGTRMTISEDTTFTYAGMFTLTTPLIGPSGMTGRSLHSSP

SEQ ID NO: 31 - protein, 2254 aa, Sapporo virus
MANCRPLPIGHLPNRIFGTPRLTPGWVWACTKEAMFKLDWLQDPVVIRQPDVYIAQGVVDDFFRPKRVLQGDPQLIAQVLLGDASGPLVGPVSMQQLTSLLHEVSQALNDHKHPLANRYTKASLQRYADTLSNYIPLVEILTGPKDLTPRDVLEQLAAGREWECVPDSALKKVFRDMWQYICEGCDSVYIKLQEVKRKIPHIDTTVLKQFFITLTDTISMATALDTKTWLAHILGWLKPTCLVMIMQQHINSPQGWAATLTALAELYYGIMPLTEVLGSIAGWVTDKFADVTTNTWGKFKAWWDNLYTPQAGNDLIILGGVVGLVYFMVFGDAPTQMFTKKLMKVCGFITSTVAAIKAAMWIVDYFKQREHEHQVRVTLARWAALQEVIKQNRCAGLSEIAKLKECCEVLLNEVTELMYKLGASPLAGLIRSTSDVIQTTINDLAQLMAYDTQRKPPAMIVFGGPPGIGKTRLVEALARQLGEVSHFTMTVDHYDTYTGNTVAIWDEFDVDSKQAFIEATIGIVNCAPYPLNCDRPEAKGRVFTSKYVLATTNCPTPVMPDHPRAMAFWRRITFIDVTAPTIEQWLIDNPGRKAPASLFKEDFSHLQCSVRGYTAYDEKGNTLSGKVARARYVSVNNLLDLIKEKYNSEATDIKHLWFTVPQSIHKQARDIILGWLRFHSYPNTVADNIPLSEVRDPTCFGYVVISDVDPPRHVSDHVAHIEVESILRTDIMDLLREGGGGLFRALKVKSAPRNCVINKVMMQAHHTTLQVLTSQEPNPPNLPRPRRLVFVESPIDIISALRHHVGFCTIPGIVKLITSGVGLGVENLGNFLQSIAGNVRFPLQSECSLLRTPGGDVLFYTSGQAAVWATPARFPIITPGEASVGKDVCSESSWWDILKALFSTLVVAFGPIATLVLTAHNLAYLNTRENTLSEAKGKNKRGRGARKAIALRDDEYDEWQDIIRDWRKEMTVQQFLDLKERALSGASDPDSQRYNAWLELRAKRLSAGAYQHAVVDIIGKSGHRREVIRTQVMRAPREPKADTYDSEGRGYVVPMTAQEKHTGWAVHIGNGRLVTCTHVANMCDHVAEVEFKITETDRDTCIITAPLGHLPSVALGDGPPAFYTTNFHPIRVLDEGSWDTTTTRVTGWRVVINNGTTTAPGDCGQPYLNARRQLVGVHAATSTCGVKKLVSRVQTKRASKATFPWKGLPVTNMPDAGGLPTGTRYHRSIAWPKQLPEETHAPAPYGVNDPRYSFSQHQMIANNLQPYTKTPVALDQTLLQRAVKHTKGYLDQVIGTHRSPNLTYAAAVDSMAHDTACGPNLPGRKKDYMTDQGEPIGPLKQMLEEAWDMAHRGVPRKHEYKLALKDELRPIEKNDQGKRRLLWGCDAGVSLVANAVFKPVAERLVDTVPMHPVAVGICMDSPQIEQMNQALTGRVLYCLDYSKWDSTQNPAVTSASVDILASYAEDTPLTSAAIATLCSPAIGRLDDIGLTVTTGLPSGMPFTSVINSINHMVYFAMAVLEAYEEFKVPYMGNIFDNETVYTYGDDCVYGLTPATASIMPVVVKNLTSYGLVPTAADKSQAIEPTDTPIFLKRTFSQTPFGLRALLDETSLARQCYWVKANRTTDLFEPAAVDVEIRKNQLEVMLAYASQHPRAVFDKLAGMVEATANAEGYQVVNVDWANAVATYNAWYGGTNGGHAPTNEDEEPEVFVMEAPAPTRSVASNPEGTQTSNESRPVQPAGPMPVATAQALEMAVATGQVNDTIPSVVRETFSTYTNVTWTTRQPAGTLLARMTLGPGLNPYTLHLSAMWAGWGGSFEIKVVISGSGLYAGKLLCALIPPGVDPSAVDQPGAFPHALVDARITEGVTFTLGDVRAVDYHETGAGGTIACLALYVYQPLINPFETALSAAMVTIETRPGPDFGFTLLKPPNQTMEAGLDPRSLLPRTARTLRGNRFGRPITGVVIVGMAYQINRHFSAAGTTLGWSTAPIGPCVGRINSRYTNNGGLAVLSMQPLSNGPLYPNIINHYPDVAASKAFNTSTTLSDSTTCGGGPMVLFNDVGDVVENVSYQMRFIASQATSQTPTLVDYINATSMGLVSFGNSRGDFGSGQLNVGVELTYTCGNTAINEKVTTFMDRQYTFGAQGPNNIMLWVETVLGTHTGNNTVYSSQPDTVSAALQGQPYNIPDGYMAVWNVNANSADFQIGLRRDGFFVTSGAIGTRMTISDDTTFTYAGMFALTTPLIGPSGMTGRSLHSSP

SEQ ID NO: 32 - RNA, 170 nt, Sapporo virus
  source 1..170: mol_type = genomic RNA
caaatgatgttttcaccaaaacacttaatgcaagcacaacacgtggtcggcactttttcccgtgggatgcctggtgtcacccccagtaagggattgcccagaccacagggcgttacagccaaagtgcctgtggctggagccaccaccactcacagcaaagtgtgattttc

SEQ ID NO: 33 - RNA, 191 nt, Sapporo virus
  source 1..191: mol_type = genomic RNA
caaatgatgttctcatcaaaacacttgatgcaagcacaaaacgtgcttggcactttttcccgtgggacacctggtgttacccccagcaaagggttgcctaaaccgcaaggcatcacccccaggccacagggcgtcacagcctccgtgcccgtggctggagtcaccaccactcacagcaaagtgtgattttc

SEQ ID NO: 34 - RNA, 200 nt, Sapporo virus
  source 1..200: mol_type = genomic RNA
caaatgatgttttcatcaaagcatctcatgcaagcgcaaaacgtgcttggcaccttttctcggggaacacctggtgtcacccccagtaaggggctgcccaaaccacagggcgtcacgtccagaccgctcagaccacagggtgtctcagccaccatacctgtggccggagccaccaccaccaacagcagagtgtgattttc

SEQ ID NO: 35 - RNA, 191 nt, Sapporo virus
  source 1..191: mol_type = genomic RNA
caaatgatgttttcatcaaaacacctgatgcaagcacaaaatgtgcttggcactttttcccgtggaacgcctggtgtcacccctagcaaaggactgcctaaaccacagggcatcaaccccaggccacagggcgtcacagcctccgtgccggtggctggagccaccaccactcacagcaaagtgtgattttc

SEQ ID NO: 36 - RNA, 191 nt, Sapporo virus
  source 1..191: mol_type = genomic RNA
cagatgatgttttcatcaaaacacctgatgcaagcacaaaatgtgctcggcactttttcccgtgggacgcctggtgtcaccccaagcaagggactgcctaaaccgcagggcatcacccccaggccacagggcgtcacagcctccgtgccggtggctggagctaccaccactcacagcaaagtgtgattttc

SEQ ID NO: 37 - protein, 19 aa, Sapporo virus
DPSAVDQPGAFPHALVDAR

SEQ ID NO: 38 - protein, 20 aa, Sapporo virus
RSLLPRTARTLRGNRFGRPI